(19) United States
(12) Patent Application Publication
(10) Pub. No.: US 2024/0398861 A1
(43) Pub. Date: Dec. 5, 2024

(54) ENHANCED CELL-DERIVED VESICLES FOR CANCER THERAPY

(71) Applicant: RESEARCH INSTITUTE AT NATIONWIDE CHILDREN'S HOSPITAL, Columbus, OH (US)

(72) Inventors: Kevin A. Cassady, Columbus, OH (US); Uksha Saini, Columbus, OH (US); Dawn Chandler, Columbus, OH (US); Ruoning Wang, Columbus, OH (US); Nicolas Wein, Columbus, OH (US)

(21) Appl. No.: 18/689,800

(22) PCT Filed: Sep. 6, 2022
(86) PCT No.: PCT/US22/42645

§ 371 Date: Mar. 6, 2024

Related U.S. Application Data
(60) Provisional application No. 63/241,497, filed on Sep. 7, 2021.

(51) Int. Cl. A61K 35/13; A61K 39/00; A61P 37/04

(52) U.S. Cl. CPC A61K 35/13; A61K 39/00114; A61K 39/001142; A61P 37/04

(57) ABSTRACT

This disclosure relates to populations and compositions of purified cancer cell-derived vesicles and uses thereof.

### CROSS-REFERENCE TO RELATED PATENT APPLICATION

This application claims the benefit under 35 U.S.C. § 119 (e) of U.S. Provisional Application No. 63/241,497, filed Sep. 7, 2021, the entire contents of which are incorporated herein by reference.

### BACKGROUND

Exosomes are nanoscale extracellular vesicles secreted by almost all kinds of eukaryotic cells or bacteria. Unlike the outward blebbing of microvesicles, the biogenesis of exosomes involves a series of complex molecular regulation and substances exchange. It is generally believed that exosomes originate from the endosomes formed by the inward budding of plasma membrane. Exosomes are various in a size range from 30 to 200 nm and exist in complex bio-systems, which provide significant challenges for the isolation and enrichment of exosomes.

Tumor cells actively produce, release, and utilize exosomes to promote tumor growth. Mechanisms through which tumor-derived exosomes subserve the tumor are under intense investigation. These exosomes are information carriers, conveying molecular and genetic messages from tumor cells to normal or other abnormal cells residing at close or distant sites. Tumor-derived exosomes are found in all body fluids. Upon contact with target cells, they alter phenotypic and functional attributes of recipients, reprogramming them into active contributors to angiogenesis, thrombosis, metastasis, and immunosuppression. Exosomes produced by tumors carry cargos that in part mimic contents of parent cells and are of potential interest as noninvasive biomarkers of cancer.

### SUMMARY OF THE DISCLOSURE

Disclosed herein are isolated extracellular vesicles (EVs) that are naturally produced by tumor or cancer cells. The EVs are useful in methods to produce an immune inert drug delivery system. This technology permits repeated treatments without fear of the immune response limiting therapeutic delivery.

This disclosure provides an isolated or purified extracellular vesicle (EV) isolated or purified from a cancer cell, wherein the EV comprises a therapeutic agent. In another aspect, the EV further comprises a surface membrane associated protein that selectively targets a cancer cell.

The EVs and compositions containing same are useful in a method for inhibiting the growth or metastasis of a cancer cell comprising, or consisting essentially of, or yet further consisting of contacting the cancer cell with an effective amount of the EVs and compositions as described herein, thereby inhibiting the growth of, or metastasis of the cancer or cancer cell. In one aspect, the contacting is in vitro or ex vivo, and can be used to test if the EV therapy, alone or in combination with other therapies, can inhibit the growth of or metastasis of the cancer or cancer cell. It also can be used in pre-clinical studies to determine effective amounts of mono- or combination therapies. The contacting can be in vivo, and provide a therapy as described herein.

The amount to be delivered is an effective amount which can be determined by the treating veterinarian or treating physician, and will vary with the cancer to be treated, the staging of the cancer, the health and age of the subject or patient. In one aspect, the EVs are isolated from cancer cells originally isolated from the subject to be treated, which are then modified to contain the EVs comprising the therapeutic agent. Alternatively, the EVs can be isolated from cultured cells from primary cell sources or commercial sources.

Also provided herein is a method of treating a cancer in a subject in need thereof comprising, or consisting essentially of, or yet further consisting of administering to the subject an effective amount of extracellular vesicles (EVs) comprising a therapeutic agent of this disclosure thereby treating the subject. In one aspect, the EVs are isolated from a cancer or cancer cells of the subject and modified to comprise the therapeutic agent. Modes of administration are described herein, and can be systemic or local to the tumor in the subject. The cancer for treatment can be primary, metastatic or relapsed.

In one aspect, provided herein is a method for treating a cancer or tumor in a subject by isolating cancer cells from the subject and modifying the cells to express a therapeutic agent in the EVs of the cells and isolating the EVs and then administering an effective amount of the EVs to the subject.

### BRIEF DESCRIPTION OF THE DRAWINGS

FIG. 1 provides a schematic overview of EV design, production and delivery.

FIGS. 2A-2B show the results of in vivo experiments using labeled EVs. (A) Representative in vivo imaging system (IVIS) images of DID (also referred to herein as DiD) labeled EVs. (B) A quantitative analysis thereof (FIG. 2B). Mice were implanted with bilateral CT2A flank gliomas (CT2A cell line is derived from a sub-cutaneous, non-metastatic murine glioma (astrocytoma) as described in Ghonime M G et al., Journal for ImmunoTherapy of Cancer, 2021; 9: e002939. doi: 10.1136/jitc-2021-002939, which is incorporated herein in its entirety). Once established and >50 mm³ in size, the tumors were injected on the left side with DID labeled and IL-21 loaded EVs prepared from CT2A tumor line (200 μg). The contralateral side was injected with PBS (no DID dye). Over 24-48h, there was increased DID detection in the contralateral untreated tumor suggesting that the EVs from the left tumor were being selectively taken up by the PBS treated tumor in the right flank.

FIG. 3 compares AAV, extracellular vesicle, microvesicle and apoptotic body.

FIGS. 4A-4D show characterizing, loading and assessing delivery of EVs isolated from CT2A murine glioma cells. (A) Nanosite and immunoblot characterization of the constructed EVs. (B) IL-21 levels measured by an ELISA assay. (C) An illustration of labeling the EVs to test their loading to CT2A cells. (D) A representative microscopy image showing the localization of the EVs.

FIGS. 5A-5B provide quantification and qualification of the EGFP-infected EV-spiked cells using flow cytometry. (A) Control experiments. (B) EGFP-infected and EGFP-infected and EV spiked cells.

FIG. 6 provides an exemplary schematic overview of detection and monitoring EVs in vivo.

FIG. 7 shows result of an in vitro fluorescence assay. Samples were diluted in 100 μl of phosphate buffered saline (PBS) and imaged using IVIS 200 small animal imaging system (Pelkin Elmer, Waltham, MA, USA). Ex filter=640 nm, Em filter=700 nm with background subtraction.

FIGS. 8A-8D show imaging results (imaging and quantification) of labeled EVs in vivo. (A) Representative IVIS images post administration. DID labeled EVs exhibit tumor selective distribution up to 5 days post injection. (B) Representative IVIS images post administration. DID-labeled EVs migrate to contralateral un-injected tumor while not targeting non-tumor sites. (C) In vivo DID fluorescence of the EV-DID treated and PBS treated tumors in (B). (D) Amount of EV protein loaded cargo (e.g., cytokine loaded EV) in injected tumor, uninjected tumor, and other tissues such as liver, blood and kidney, as determined by direct measurement 36 hours post administration.

FIGS. 9A-9C show EV organ distribution after administration. (A) A representative fluorescence image showing biodistribution of DiD labelled exosomes in various organs after sacrifice, indicating the DiD-labelled EVs migrate to another tumor but not non-tumor tissues, such as liver, spleen, kidneys and omentum. (B) Testing delivery of an expression cassette (in this case, AAV-GFP, AAV serotype 9) using EVs. (C) RT-PCR of gene expression (GFP) in liver and kidney 14 days after EV delivery. The AAV-GFP gene expression was not detectable in either organs, suggesting that gene delivery by EVs of the instant disclosure is tumor-specific.

FIG. 10 shows EVs migrate to contralateral un-injected tumor.

FIGS. 11A-11B shows results of experiments delivering gene expression cassettes (DNA) using EVs. (A) EVs loaded with human interleukin 21 (hIL21) gene expression cassette produces hIL21 both in vitro and in vivo. (B) EVs loaded with human interleukin 21 (hIL21) and human CXCL10 expression cassettes can produce hIL21 and CXCL10 both in vitro and in vivo. In both sets of experiments, the expressed genes were also detected in the uninjected tumor sites, indicating that EVs can travel and deliver their payload to the uninjected site as well.

FIGS. 12A-12C show EVs expressing C—X—C Motif Chemokine Ligand 10 (CXCL10) and interleukin 21 (IL21 or IL-21) improve recruiting NK cells to tumors. (A) NK events measured by flow cytometry 2 days after treatment with EVs. (B) CD45-gated NK cells measured by flow cytometry 2 days after treatment with EVs. (C) Tumor size 12 days post EV treatment.

FIGS. 13A-13B show EVs expressing CXCL10 and IL21 suppress tumor growth and improve survival. (A) NK cells percentage detected in each of the cohorts. (B) The tumor sizes upon various treatments. It is noted that tumors continued to grow in the control EVs or PBS groups and they met with the tumor burden sacrifice criterion within 15 days of injection and had to be sacrificed.

FIGS. 14A-14F show that survivors after the treatment by the EVs expressing CXCL10 and IL21 developed anti-tumor specific T cell immunity. (A) Percent of CD8 splenocytes that are reactive to OVA, EphA2 or huClip antigens in survivors that have undergone IL21 containing EV treatment. (B) Percent of all CD8 splenocytes that are reactive to EphA2 under different treatments. (C) Percent of all CD8 splenocytes that are reactive to OVA under different treatments. (D) Percent of CD4 splenocytes that are reactive to OVA, EphA2 or huClip antigens in survivors that have undergone IL21 containing EV treatment. (E) Percent of all CD4 splenocytes that are reactive to EphA2 under different treatments. (F) Percent of all CD4 splenocytes that are reactive to OVA under different treatments.

FIGS. 15A-15C show that survivors after the treatment by the EVs expressing CXCL10 and IL21 had better memory response than those after the treatment by cytokine alone. Mice treated with EV-CXCL10+IL21 (either unilateral or bilateral injection) had improved T cell memory to the tumor antigen than mice treated with bilateral injections of EV-IL21 or C021, an oncolytic virus expressing IL21. (A) Percent of all CD8 splenocytes that are reactive to EphA2 under different treatments. (B) Percent of all CD8 splenocytes that are reactive to OVA under different treatments. (C) CD8-EphA2 responsive cells of all splenocytes.

FIG. 16 provides an exemplary schematic overview of orthotopic model workflow.

FIG. 17 shows generation of adeno-associated viruses (AAVs) capsulated in EVs and uses thereof in delivering a gene of interest to cells in vitro.

FIG. 18 shows generation of AAVs capsulated in EVs and uses thereof in delivering a gene of interest to tumor cells in vitro.

FIG. 19 provides a schematic overview of investigating uses of AA Vs capsulated in EVs in transferring a gene of interest from infected tumor cells to progeny cells thereof.

FIG. 20 shows uses of AAVs capsulated in EVs in transferring a gene of interest from infected tumor cells to progeny cells thereof.

FIG. 21 shows uses of AAVs capsulated in EVs in delivering a gene of interest to tumor cells in vivo.

FIG. 22 shows uses of EVs in delivering a splice-switching oligonucleotide (SSO).

FIGS. 23A-23E show that EV-delivered immunotherapeutic elicits an antitumor response in the contralateral tumor CT2A. (A) In vivo imaging shows that PBS-treated contralateral tumors exhibit DID signal, showing that the EV-delivered DID relocates to uninjected sites. (B) Absolute tumor growth under different treatments. (C) Bilateral tumor growth after control or experimental EV treatment. (D) Tumor growth after control or experimental EV treatment in the injected site. (E) Tumor growth after control or experimental EV treatment in the uninjected site.

FIGS. 24A-24E show that EV-delivered immunotherapeutic elicits an antitumor response in the contralateral tumor 67C4. (A) Tumor size measurements under different EC or oncolytic virus treatments. (B)—(C) Tumor size under Oncolytic virus treatment. (D)-(E) Tumor size under control or IL-21 containing EVs in the injected and uninjected sites.

FIGS. 25A-25B show that (A) EV contents are delivered to both injected and uninjected tumor sites and (B) NK cells infiltrate both injected and uninjected tumor sites.

FIGS. 26A-26B show that IL21 improves oncolytic virus (OV) response in CT2A brain tumors. CT2A cells were injected to mice intracranially (IC). Seven days later tumors were treated and survival was observed over 50-60 days. (A) Oncolytic virus expressing hIL21 treated mice survived longer than saline-treated mice. (B) Oncolytic virus expressing hIL21/mCXCL10 treated mice survived longer than saline-treated mice.

FIGS. 27A-27C show that therapeutic EVs can cross the blood brain barrier in treating intracranial (IC) tumors. (A) Survival plots after saline or 250 pg hIL21+hCXCL10 intracranial EV administration. (B) Survival plots after saline or 500 pg hIL21+hCXCL10 intracranial EV administration. (C) Survival plots after saline or 250 pg hIL21+hCXCL10 intraperitoneal EV administration.

FIGS. 28A-28E show that EVs can cross the blood-brain barrier. (A) Experimental setup for intracranial and intraperitoneal treatment with EVs. (B) Orthotopic CT2A tumor mice were treated either IC or IP. Brains harvested from the mice sacrificed at D6 post treatment were used for flow cytometric analyses. The mice treated with EVhCXCL10-IL21 had significantly higher CD45CD8+ cells as compared to saline or unloaded EV control treated mice (combined IP and IC EVCTRL treated mice). (C) The CD8 influx for C021 treated mice compared to EV treatments (control, IP or IC). (D) C021 (C134+hIL21) IC inoculation elicits CD8 T cell brain infiltrates. (E) EVs loaded withCXCL10 & IL21 increase CD8 T cell infiltration. IP treatment induces similar CD8 infiltrates as direct IC injection of the EVs. These results suggest that EVs can cross the blood brain barrier to find the tumor like a therapeutic “homing pigeon.”

FIGS. 29A-29B show that EVs can cloak a naked virus (AAV), allowing its entry, integration and gene expression (A) in vitro and (B) in vivo. The experiments show a dose response effect and also suggest transfer to contralateral (un-injected) tumors.

FIG. 30 shows that EVs isolated from transduced tumor cells can transfer a phenotype (in this instance expressing GFP) to other tumor cells. These results suggest that an EV-based therapy can be propagated between tumor cells

### DETAILED DESCRIPTION

It is to be understood that this invention is not limited to particular embodiments described, as such may, of course, vary. It is also to be understood that the terminology used herein is for the purpose of describing particular embodiments only, and is not intended to be limiting, since the scope of this invention will be limited only by the appended claims.

The detailed description of the invention is divided into various sections only for the reader's convenience and disclosure found in any section may be combined with that in another section. Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated by reference to disclose and describe the methods and/or materials in connection with which the publications are cited.

All numerical designations, e.g., pH, temperature, time, concentration, and molecular weight, including ranges, are approximations which are varied (+) or (−) by increments of 0.1 or 1.0, where appropriate. It is to be understood, although not always explicitly stated, that all numerical designations are preceded by the term “about.” It also is to be understood, although not always explicitly stated, that the reagents described herein are merely exemplary and that equivalents of such are known in the art.

It must be noted that as used herein and in the appended claims, the singular forms “a”, “an”, and “the” include plural referents unless the context clearly dictates otherwise. Thus, for example, reference to “a cell” includes a plurality of cells.

As will be understood by one skilled in the art, for any and all purposes, all ranges disclosed herein also encompass any and all possible subranges and combinations of subranges thereof. Furthermore, as will be understood by one skilled in the art, a range includes each individual member.

The practice of the present disclosure will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Techique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3rd edition (Cold Spring Harbor Laboratory Press (2002)); Sohail (ed.) (2004) Gene Silencing by RNA Interference: Technology and Application (CRC Press).

### Definitions

As it would be understood, the section or subsection headings as used herein is for organizational purposes only and are not to be construed as limiting and/or separating the subject matter described.

The following definitions assist in defining the meets and bounds of the inventions as described herein. Unless specifically noted, the embodiments describing “cell-derived vesicles” shall include “exosomes,” “microvesicles” alone or in combination.

The term “about” when used before a numerical designation, e.g., temperature, time, amount, concentration, and such other, including a range, indicates approximations which may vary by (+) or (−) 10%, 5% or 1%.

The terms “administering” or “administration” in reference to delivering cell-derived vesicles to a subject include any route of introducing or delivering to a subject the cell-derived vesicles to perform the intended function. Administration can be carried out by any suitable route, including orally, intranasally, parenterally (intravenously, intramuscularly, intraperitoneally, or subcutaneously), intracranially, or topically. Additional routes of administration include intraorbital, infusion, intraarterial, intracapsular, intracardiac, intradermal, intrapulmonary, intraspinal, intrasternal, intrathecal, intrauterine, intravenous, subarachnoid, subcapsular, subcutaneous, transmucosal, or transtracheal. Administration includes self-administration and the administration by another.

“Comprising” or “comprises” is intended to mean that the compositions, for example media, and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

As used herein, comparative terms as used herein, such as high, low, increase, decrease, reduce, or any grammatical variation thereof, can refer to certain variation from the reference. In some embodiments, such variation can refer to about 10%, or about 20%, or about 30%, or about 40%, or about 50%, or about 60%, or about 70%, or about 80%, or about 90%, or about 1 fold, or about 2 folds, or about 3 folds, or about 4 folds, or about 5 folds, or about 6 folds, or about 7 folds, or about 8 folds, or about 9 folds, or about 10 folds, or about 20 folds, or about 30 folds, or about 40 folds, or about 50 folds, or about 60 folds, or about 70 folds, or about 80 folds, or about 90 folds, or about 100 folds or more higher than the reference. In some embodiments, such variation can refer to about 1%, or about 2%, or about 3%, or about 4%, or about 5%, or about 6%, or about 7%, or about 8%, or about 0%, or about 10%, or about 20%, or about 30%, or about 40%, or about 50%, or about 60%, or about 70%, or about 75%, or about 80%, or about 85%, or about 90%, or about 95%, or about 96%, or about 97%, or about 98%, or about 99% of the reference.

“Optional” or “optionally” means that the subsequently described circumstance may or may not occur, so that the description includes instances where the circumstance occurs and instances where it does not.

As used herein, “and/or” refers to and encompasses any and all possible combinations of one or more of the associated listed items, as well as the lack of combinations when interpreted in the alternative (“or”).

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

The terms or “acceptable,” “effective,” or “sufficient” when used to describe the selection of any components, ranges, dose forms, etc. disclosed herein intend that said component, range, dose form, etc. is suitable for the disclosed purpose.

As used herein, the term “modified,” relative to cell-derived vesicles, refers to cell-derived vesicles (e.g., exosomes and/or microvesicles) that have been altered such that they differ from a naturally occurring cell-derived vesicles. Non-limiting examples of a modified cell-derived vesicle include an exosome and/or microvesicle that contains a nucleic acid or protein of a type or in an amount different than that found in a naturally occurring exosome and/or microvesicle.

The term “purified population” or “isolated” relative to cell-derived vesicles, as used herein refers to plurality of cell-derived vesicles that have undergone one or more processes of selection for the enrichment or isolation of the desired exosome population relative to some or all of some other component with which cell-derived vesicles are normally found in culture media. Alternatively, “purified” can refer to the removal or reduction of residual undesired components found in the conditioned media (e.g., cell debris, soluble proteins, etc.). A “highly purified population” as used herein, refers to a population of cell-derived vesicles in which at least 65%, at least 70%, at least 75%, at least 80%, at least 85%, at least 90%, at least 95%, at least 98%, at least 99% or 100% of cell debris and soluble proteins (e.g., proteins derived from fetal bovine serum and the like) in the conditioned media along with the cell-derived vesicles are removed.

The term “subject,” “host,” “individual,” and “patient” are as used interchangeably herein to refer to animals, typically mammalian animals. Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human. In some embodiments, a subject has, or is diagnosed of having, or is suspected of having, or is at risk of having a disease, such as a cancer.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of this technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, treatment excludes prophylaxis.

When the disease is cancer, the following clinical endpoints are non-limiting examples of treatment: (1) elimination of a cancer in a subject or in a tissue/organ of the subject or in a cancer loci; (2) reduction in tumor burden (such as number of cancer cells, number of cancer foci, number of cancer cells in a foci, size of a solid cancer, concentrate of a liquid cancer in the body fluid, and/or amount of cancer in the body); (3) stabilizing or delay or slowing or inhibition of cancer growth and/or development, including but not limited to, cancer cell growth and/or division, size growth of a solid tumor or a cancer loci, cancer progression, and/or metastasis (such as time to form a new metastasis, number of total metastases, size of a metastasis, as well as variety of the tissues/organs to house metastatic cells); (4) less risk of having a cancer growth and/or development; (5) inducing an immune response of the patient to the cancer, such as higher number of tumor-infiltrating immune cell, higher number of activated immune cells, or higher number cancer cell expressing an immunotherapy target, or higher level of expression of an immunotherapy target in a cancer cell; (6) higher probability of survival and/or increased duration of survival, such as increased overall survival (OS, which may be shown as 1-year, 2-year, 5-year, 10-year, or 20-year survival rate), increased progression free survival (PFS), increased disease free survival (DFS), increased time to tumor recurrence (TTR) and increased time to tumor progression (TTP). In some embodiments, the subject after treatment experiences one or more endpoints selected from tumor response, reduction in tumor size, reduction in tumor burden, increase in overall survival, increase in progression free survival, inhibiting metastasis, improvement of quality of life, minimization of drug-related toxicity, and avoidance of side-effects (e.g., decreased treatment emergent adverse events). In some embodiments, improvement of quality of life includes resolution or improvement of cancer-specific symptoms, such as but not limited to fatigue, pain, nausea/vomiting, lack of appetite, and constipation; improvement or maintenance of psychological well-being (e.g., degree of irritability, depression, memory loss, tension, and anxiety); improvement or maintenance of social well-being (e.g., decreased requirement for assistance with eating, dressing, or using the restroom; improvement or maintenance of ability to perform normal leisure activities, hobbies, or social activities; improvement or maintenance of relationships with family). In some embodiments, improved patient quality of life that is measured qualitatively through patient narratives or quantitatively using validated quality of life tools known to those skilled in the art, or a combination thereof. Additional non-limiting examples of endpoints include reduced hospital admissions, reduced drug use to treat side effects, longer periods off-treatment, and earlier return to work or caring responsibilities. In one aspect, prevention or prophylaxis is excluded from treatment.

In certain embodiments, the terms “disease” “disorder” and “condition” are used interchangeably herein, referring to a cancer, a status of being diagnosed with a cancer, or a status of being suspect of having a cancer. “Cancer”, which is also referred to herein as “tumor”, is a known medically as an uncontrolled division of abnormal cells in a part of the body, benign or malignant. In one embodiment, cancer refers to a malignant neoplasm, a broad group of diseases involving unregulated cell division and growth, and invasion to nearby parts of the body. Non-limiting examples of cancers include carcinomas, sarcomas, leukemia and lymphoma, e.g., colon cancer, colorectal cancer, rectal cancer, gastric cancer, esophageal cancer, head and neck cancer, breast cancer, brain cancer, lung cancer, stomach cancer, liver cancer, gall bladder cancer, or pancreatic cancer. In one embodiment, the term “cancer” refers to a solid tumor, which is an abnormal mass of tissue that usually does not contain cysts or liquid areas, including but not limited to, sarcomas, carcinomas, and certain lymphomas (such as Non-Hodgkin's lymphoma). In another embodiment, the term “cancer” refers to a liquid cancer, which is a cancer presenting in body fluids (such as, the blood and bone marrow), for example, leukemias (cancers of the blood) and certain lymphomas.

Additionally or alternatively, a cancer may refer to a local cancer (which is an invasive malignant cancer confined entirely to the organ or tissue where the cancer began), a metastatic cancer (referring to a cancer that spreads from its site of origin to another part of the body), a non-metastatic cancer, a primary cancer (a term used describing an initial cancer a subject experiences), a secondary cancer (referring to a metastasis from primary cancer or second cancer unrelated to the original cancer), an advanced cancer, an unresectable cancer, or a recurrent cancer. As used herein, an advanced cancer refers to a cancer that had progressed after receiving one or more of: the first line therapy, the second line therapy, or the third line therapy.

The term “contacting” means direct or indirect binding or interaction between two or more. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

As used herein, the term “administration” and “administering” are used to mean introducing an agent into a subject. Routes of administration include, but are not limited to, oral (such as a tablet, capsule or suspension), topical, transdermal, intranasal, vaginal, rectal, subcutaneous intravenous, intravenous, intraarterial, intramuscular, intraosseous, intraperitoneal, intraocular, subconjunctival, sub-Tenon's, intravitreal, retrobulbar, intracameral, intratumoral, epidural and intrathecal.

An “immunotherapy agent” means a type of cancer treatment which uses a patient's own immune system to fight cancer, including but not limited to a physical intervene, a chemical substance, a biological molecule or particle, a cell, a tissue or organ, or any combinations thereof, enhancing or activating or initiating a patient's immune response against cancer. Non-limiting examples of immunotherapy agents include antibodies, immune regulators, checkpoint inhibitors, an antisense oligonucleotide (ASO), a RNA interference (RNAi), a Clustered Regularly Interspaced Short Palindromic Repeat (CRISPR) system, a viral vector, an anti-cancer cell therapy (e.g., transplanting an anti-cancer immune cell optionally amplified and/or activated in vivo, or administering an immune cell expressing a chimeric antigen receptor (CAR)), a CAR therapy, and cancer vaccines. As used herein, unless otherwise specified, an immunotherapy agent is not an inhibitor of thymidylate biosynthesis, or an anthracycline or other topoisomerase II inhibitor. As used herein, immune checkpoint refers to a regulator and/or modulator of the immune system (such as an immune response, an anti-tumor immune response, a nascent anti-tumor immune response, an anti-tumor immune cell response, an anti-tumor T cell response, and/or an antigen recognition of T cell receptor in the process of immune response). Their interaction activates either inhibitory or activating immune signaling pathways. Thus a checkpoint may contain one of the two signals: a stimulatory immune checkpoint that stimulates an immune response, and an inhibitory immune checkpoint inhibiting an immune response. In some embodiments, the immune checkpoint is crucial for self-tolerance, which prevents the immune system from attacking cells indiscriminately. However, some cancers can protect themselves from attack by stimulating immune checkpoint targets. In some embodiments, the immune checkpoints are present on T cells, antigen-presenting cells (APCs) and/or tumor cells.

One target of an immunotherapy agent is a tumor-specific antigen while the immunotherapy directs or enhances the immune system to recognize and attack tumor cells. Non-limiting examples of such agent includes a cancer vaccine presenting a tumor-specific antigen to the patient's immune system, a monoclonal antibody or an antibody-drug conjugate specifically binding to a tumor-specific antigen, a bispecific antibody specifically binding to a tumor-specific antigen and an immune cell (such as a T-cell engager or a NK-cell engager), an immune cell (such as a killer cell) specifically binding to a tumor-specific antigen (such as a CAR-T cell, a CAR-NK cell, and a CAR-NKT cell), a polynucleotide (or a vector comprising the same) transfecting/transducing an immune cell to express an tumor-specific antibody of an antigen binding fragment thereof (such as a CAR), or a polynucleotide (or a vector comprising the same) transfecting/transducing a cancer cell to express an antigen or a marker which can be recognized by an immune cell.

Another exemplified target is an inhibitory immune checkpoint which suppresses the nascent anti-tumor immune response, such as A2AR, B7-H3, B7-H4, BTLA, CTLA-4, CTLA-4/B7-1/B7-2, IDO, KIR, LAG3, NOX2, PD-1, PD-L1 and TIM-3, VISTA, SIGLEC7 (Sialic acid-binding immunoglobulin-type lectin 7, also designated as CD328) and SIGLEC9 (Sialic acid-binding immunoglobulin-type lectin 9, also designated as CD329). Non-limiting examples of such agent includes an antagonist or inhibitor of an inhibitory immune checkpoint, an agent reducing the expression and/or activity of an inhibitory immune checkpoint (such as via an antisense oligonucleotide (ASO), a RNA interference (RNAi), or a Clustered Regularly Interspaced Short Palindromic Repeat (CRISPR) system), an antibody or an antibody-drug conjugate or a ligand specifically binding to and reducing (or inhibiting) the activity of an inhibitory immune checkpoint, an immune cell with reduced (or inhibited) an inhibitory immune checkpoint (and optionally specifically binding to a tumor-specific antigen, such as a CAR-T cell, a CAR-NK cell, and a CAR-NKT cell), and a polynucleotide (or a vector comprising the same) transfecting/transducing an immune cell or a cancer cell to reduce or inhibit an inhibitory immune checkpoint thereof. Reducing expression or activity of such inhibitory immune checkpoint enhances immune response of a patient to a cancer.

A further possible immunotherapy target is a stimulatory checkpoint molecule (including but not limited to 4-1BB, CD27, CD28, CD40, CD122, CD137, OX40, GITR and ICOS), wherein the immunotherapy agent actives or enhances the anti-tumor immune response. Non-limiting examples of such agent includes an agonist of a stimulatory checkpoint, an agent increasing the expression and/or activity of a stimulating immune checkpoint, an antibody or an antibody-drug conjugate or a ligand specifically binding to and activating or enhancing the activity of a stimulating immune checkpoint, an immune cell with increased expression and/or activity of a stimulating immune checkpoint (and optionally specifically binding to a tumor-specific antigen, such as a CAR-T cell, a CAR-NK cell, and a CAR-NKT cell), and a polynucleotide (or a vector comprising the same) transfecting/transducing an immune cell or a cancer cell to express a stimulating immune checkpoint thereof.

Additional or alternative targets may be utilized by an immunotherapy agent, such as an immune regulating agent, including but not limited to, an agent activating an immune cell, an agent recruiting an immune cell to a cancer or a cancer cell, or an agent increasing immune cell infiltrated into a solid tumor and/or a cancer loci. Non-limiting examples of such agent is an immune regulator or a variant, a mutant, a fragment, an equivalent thereof.

In some embodiments, an immunotherapy agent utilizes one or more targets, such as a bispecific T cell engager, a bispecific NK cell engager, or a CAR cell therapy. In some embodiments, the immunotherapy agent targets one or more immune regulatory or effector cells.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits, rat, canine, donkey, mice, camelids (such as dromedaries, llamas, and alpacas), as well as non-mammalian species, such as shark immunoglobulins. Unless specifically noted otherwise, the term “antibody” includes intact immunoglobulins and “antibody fragments” or “antigen binding fragments” that specifically bind to a molecule of interest (or a group of highly similar molecules of interest) to the substantial exclusion of binding to other molecules (for example, antibodies and antibody fragments that have a binding constant for the molecule of interest that is at least 103 M-1 greater, at least 10+M-1 greater or at least 105 M-1 greater than a binding constant for other molecules in a biological sample). The term “antibody” also includes genetically engineered forms such as chimeric antibodies (for example, murine or humanized non-primate antibodies), heteroconjugate antibodies (such as, bispecific antibodies). See also, Pierce Catalog and Handbook, 1994-1995 (Pierce Chemical Co., Rockford, Ill.); Owen et al., Kuby Immunology, 7th Ed., W. H. Freeman & Co., 2013; Murphy, Janeway's Immunobiology, 8th Ed., Garland Science, 2014; Male et al., Immunology (Roitt), 8th Ed., Saunders, 2012; Parham, The Immune System, 4th Ed., Garland Science, 2014. The term “antibody” includes any protein or peptide containing molecule that comprises at least a portion of an immunoglobulin molecule, such as the whole antibody and any antigen binding fragment or a single chain thereof. The terms “antibody,” “antibodies” and “immunoglobulin” also include immunoglobulins of any isotype, fragments of antibodies which retain specific binding to antigen, including, but not limited to, Fab, Fab′, F (ab) 2, Fv, scFv, dsFv, Fd fragments, dAb, VH, VL, VhH, and V-NAR domains; minibodies, diabodies, triabodies, tetrabodies and kappa bodies; multispecific antibody fragments formed from antibody fragments and one or more isolated. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, at least one portion of a binding protein, chimeric antibodies, humanized antibodies, single-chain antibodies, and fusion proteins comprising an antigen-binding portion of an antibody and a non-antibody protein. The variable regions of the heavy and light chains of the immunoglobulin molecule contain a binding domain that interacts with an antigen. The constant regions of the antibodies (Abs) may mediate the binding of the immunoglobulin to host tissues. The antibodies can be polyclonal, monoclonal, multispecific (e.g., bispecific antibodies), and antibody fragments, so long as they exhibit the desired biological activity.

As used herein, the term “monoclonal antibody” refers to an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

In some embodiments, the antibody is a bispecific immune cell engager, referring to a bispecific monoclonal antibody that is capable of recognizing and specifically binding to a tumor antigen (such as CD19, EpCAM, MCSP, HER2, EGFR or CS-1) and an immune cell, and directing an immune cell to cancer cells, thereby treating a cancer. Non-limiting examples of such antibody include bispecific T cell engager, bispecific cytotoxic T lymphocytes (CTL) engager, and bispecific NK cell engager. In one embodiment, the engager is a fusion protein consisting of two single-chain variable fragments (scFvs) of different antibodies. Additionally or alternatively, the immune cell is a killer cell, including but not limited to: a cytotoxic T cell, a gamma delta T cell, a NK cell and a NK-T cell.

As used herein, the term “antigen binding domain” refers to any protein or polypeptide domain that can specifically bind to an antigen target.

The term “chimeric antigen receptor” (CAR), as used herein, refers to a fused protein comprising an extracellular domain capable of binding to an antigen, a transmembrane domain derived from a polypeptide different from a polypeptide from which the extracellular domain is derived, and at least one intracellular domain. The “chimeric antigen receptor (CAR)” is sometimes called a “chimeric receptor”, a “T-body”, or a “chimeric immune receptor (CIR).” The “extracellular domain capable of binding to an antigen” means any oligopeptide or polypeptide that can bind to a certain antigen. The “intracellular domain” or “intracellular signaling domain” means any oligopeptide or polypeptide known to function as a domain that transmits a signal to cause activation or inhibition of a biological process in a cell. In certain embodiments, the intracellular domain may comprise, alternatively consist essentially of, or yet further comprise one or more costimulatory signaling domains in addition to the primary signaling domain. The “transmembrane domain” means any oligopeptide or polypeptide known to span the cell membrane and that can function to link the extracellular and signaling domains. A chimeric antigen receptor may optionally comprise a “hinge domain” which serves as a linker between the extracellular and transmembrane domains.

As used herein, a CAR therapy may refer to administrating an immune cell expressing a CAR into a subject as well as contacting a vector expressing a CAR in an immune cell (such as in vivo).

“Immune response” broadly refers to the antigen-specific or non-specific responses of lymphocytes to foreign substances. The terms “immunogen” and “immunogenic” refer to molecules with the capacity to elicit an immune response. All immunogens are antigens, however, not all antigens are immunogenic. An immune response disclosed herein can be humoral (via antibody activity) or cell-mediated (via T cell activation). The response may occur in vivo or in vitro. The skilled artisan will understand that a variety of macromolecules, including proteins, nucleic acids, fatty acids, lipids, lipopolysaccharides and polysaccharides have the potential to be immunogenic. The skilled artisan will further understand that nucleic acids encoding a molecule capable of eliciting an immune response necessarily encode an immunogen. The artisan will further understand that immunogens are not limited to full-length molecules, but may include partial molecules.

As used herein, “immune cells” includes, e.g., white blood cells (leukocytes, such as granulocytes (neutrophils, eosinophils, and basophils), monocytes, lymphocytes (T cells, B cells, natural killer (NK) cells and NKT cells)), and myeloid-derived cells (neutrophil, eosinophil, basophil, monocyte, macrophage, and dendritic cells). In some embodiments, the immune cell comprises, or alternatively consists essentially of, or yet further consists of one or more of: an NK cell, an NKT cell, a T cell, a B cell, a dendritic cell, a splenocyte or a macrophage.

As used herein, the term “NK cell,” also known as natural killer cell, refers to a type of lymphocyte that originates in the bone marrow and play a critical role in the innate immune system. NK cells provide rapid immune responses against viral-infected cells, tumor cells or other stressed cell, even in the absence of antibodies and major histocompatibility complex on the cell surfaces. NK cells for using in a cell therapy and/or a CAR therapy may either be isolated or obtained from a commercially available source. Non-limiting examples of commercial NK cell lines include lines NK-92 (ATCC® CRL-2407™), NK-92 MI (ATCC® CRL-2408™). Further examples include but are not limited to NK lines HANK1, KHYG-1, NKL, NK—YS, NOI-90, and YT. Non-limiting exemplary sources for such commercially available cell lines include the American Type Culture Collection, or ATCC, (www.atcc.org/) and the German Collection of Microorganisms and Cell Cultures (www.dsmz.de/).

As used herein, the term “T cell,” refers to a type of lymphocyte that matures in the thymus. T cells play an important role in cell-mediated immunity and are distinguished from other lymphocytes, such as B cells, by the presence of a T-cell receptor on the cell surface. T-cells for using in a cell therapy and/or a CAR therapy may either be isolated or obtained from a commercially available source. “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses. Non-limiting examples of commercially available T-cell lines include lines BCL2 (AAA) Jurkat (ATCC® CRL-2902™), BCL2 (S70A) Jurkat (ATCC® CRL-2900™), BCL2 (S87A) Jurkat (ATCC® CRL-2901™), BCL2 Jurkat (ATCC® CRL-2899™), Neo Jurkat (ATCC® CRL-2898™), TALL-104 cytotoxic human T cell line (ATCC #CRL-11386). Further examples include but are not limited to mature T-cell lines, e.g., such as Deglis, EBT-8, HPB-MLp-W, HUT 78, HUT 102, Karpas 384, Ki 225, My-La, Se-Ax, SKW-3, SMZ-1 and T34; and immature T-cell lines, e.g., ALL-SIL, Be13, CCRF-CEM, CML-T1, DND-41, DU.528, EU-9, HD-Mar, HPB-ALL, H—SB2, HT-1, JK-T1, Jurkat, Karpas 45, KE-37, KOPT-K1, K-T1, L-KAW, Loucy, MAT, MOLT-1, MOLT 3, MOLT-4, MOLT 13, MOLT-16, MT-1, MT-ALL, P12/Ichikawa, Peer, PER0117, PER-255, PF-382, PFI-285, RPMI-8402, ST-4, SUP-T1 to T14, TALL-1, TALL-101, TALL-103/2, TALL-104, TALL-105, TALL-106, TALL-107, TALL-197, TK-6, TLBR-1, -2, -3, and -4, CCRF-HSB-2 (CCL-120.1), J.RT3-T3.5 (ATCC TIB-153), J45.01 (ATCC CRL-1990), J.CaM1.6 (ATCC CRL-2063), RS4; 11 (ATCC CRL-1873), CCRF-CEM (ATCC CRM-CCL-119); and cutaneous T-cell lymphoma lines, e.g., HuT78 (ATCC CRM-TIB-161), MJ [G11] (ATCC CRL-8294), HuT102 (ATCC TIB-162). Null leukemia cell lines, including but not limited to REH, NALL-1, KM-3, L92-221, are another commercially available source of immune cells for using in a CAR therapy, as are cell lines derived from other leukemias and lymphomas, such as K562 erythroleukemia, THP-1 monocytic leukemia, U937 lymphoma, HEL erythroleukemia, HL60 leukemia, HMC-1 leukemia, KG-1 leukemia, U266 myeloma. Non-limiting exemplary sources for such commercially available cell lines include the American Type Culture Collection, or ATCC, (www.atcc.org/) and the German Collection of Microorganisms and Cell Cultures (www.dsmz.de/).

As used herein, the term “splenocyte” refers to different white blood cell types in the spleen or purified from splenic tissue.

As used herein, a “cell surface marker” refers to a molecule (e.g., a protein) that is in contact with a cell and at least partially at the surface of the cell, and can be detected to distinguish the cell from one or more other types of cell from the same individual. In some embodiments, the presence and/or absence of a cell surface marker can be used to distinguish the cell from other types of cells. In some embodiments, the quantity of cell surface marker present on the surface of a cell can be used to distinguish the cell from other types of cells. In some embodiments, the cell surface marker is a transmembrane protein or a lipid-anchored protein. In some embodiments, the cell surface marker can be used to distinguish an immune cell from a non-immune cell.

Cluster of Differentiation (CD) 3 (CD3) is a protein complex and T cell co-receptor that is involved in activating both the cytotoxic T cell (CD8+ naive T cells) and T helper cells (CD4+ naive T cells) and composed of four distinct chains. It has been used as a negative marker for NK cells. Suitable antibodies for detecting such protein is available to one of skill in the art, such as from abcam (e.g., ab135372), BIOLEGEND® (e.g., UCHT1), or Thermo Fisher Scientific (e.g., 14-0037-82).

CD28 (Cluster of Differentiation 28) is one of the proteins expressed on T cells that provide co-stimulatory signals required for T cell activation and survival. T cell stimulation through CD28 in addition to the T-cell receptor (TCR) can provide a potent signal for the production of various interleukins (IL-6 in particular). CD28 is also the receptor for CD80 (B7.1) and CD86 (B7.2) proteins. When activated by Toll-like receptor ligands, the CD80 expression is upregulated in antigen-presenting cells (APCs). In some embodiments, the CD28 is a human CD28. Non-limiting exemplary sequences of this protein or the underlying gene can be found under Gene Cards ID: GC02P203706, HGNC: 1653, NCBI Entrez Gene: 940, Ensembl: ENSG00000178562, OMIM®: 186760, or UniProtKB/Swiss-Prot: P10747, each of which is incorporated by reference herein in its entirety.

The terms “4-1BBL,” “Tumor Necrosis Factor Superfamily Member 9,” “TNFSF9,” or “4-1BBL polypeptide” is a type 2 transmembrane glycoprotein receptor that is found on APCs (antigen presenting cells) and binds to 4-1BB (also known as CD137). The 4-1BB/4-1BBL complex belongs to the TNFR:TNF superfamily, which is expressed on activated T Lymphocytes. In some embodiments, the 4-1BBL is a human 4-1BBL. Non-limiting exemplary sequences of this protein or the underlying gene can be found under Gene Cards ID: GC19P006531, HGNC: 11939, NCBI Entrez Gene: 8744, Ensembl: ENSG00000125657, OMIM®: 606182, or UniProtKB/Swiss-Prot: P41273, each of which is incorporated by reference herein in its entirety.

CD16, also known as FcγRIII, is a cluster of differentiation molecule found on the surface of natural killer cells, neutrophils, monocytes, and macrophages and a molecule of the immunoglobulin superfamily (IgSF) involved in antibody-dependent cellular cytotoxicity (ADCC). CD16 has been identified as Fc receptors FcγRIIIa (CD16a) and FcγRIIIb (CD16b), which participate in signal transduction. In some embodiments, the Cd16 is a human CD16. Non-limiting exemplary sequences of CD16a or the underlying gene or suitable antibodies for detection of the protein can be found under Gene Cards ID: GC01M161541, HGNC: 3619, NCBI Entrez Gene: 2214, Ensembl: ENSG00000203747, OMIM®: 146740, or UniProtKB/Swiss-Prot: P08637, each of which is incorporated by reference herein in its entirety. Non-limiting exemplary sequences of CD16b or the underlying gene can be found under Gene Cards ID: GC01M161623, HGNC: 3620, NCBI Entrez Gene: 2215, Ensembl: ENSG00000162747, OMIM®: 610665, or UniProtKB/Swiss-Prot: 075015, each of which is incorporated by reference herein in its entirety.

NKG2D is an activating receptor on the NK cell surface. In some embodiments, the NKG2D is a human NKG2D. Non-limiting exemplary sequences of this protein or the underlying gene or suitable antibodies for detection of the protein can be found under Gene Cards ID: GC12M015056, HGNC: 18788, NCBI Entrez Gene: 22914, Ensembl: ENSG00000213809, OMIM®: 611817, or UniProtKB/Swiss-Prot: P26718, each of which is incorporated by reference herein in its entirety.

CD64 (Cluster of Differentiation 64) is a type of integral membrane glycoprotein known as an Fc receptor that binds monomeric IgG-type antibodies with high affinity. It is more commonly known as Fc-gamma receptor 1 (FcγRI). After binding IgG, CD64 interacts with an accessory chain known as the common γ chain (γ chain), which possesses an ITAM motif that is necessary for triggering cellular activation. In some embodiments, the CD64 is a human CD64. Non-limiting exemplary sequences of this protein or the underlying gene or suitable antibodies for detection of the protein can be found under Gene Cards ID: GC01P149754, HGNC: 3613, NCBI Entrez Gene: 2209, Ensembl: ENSG00000150337, OMIM®: 146760, or UniProtKB/Swiss-Prot: P12314, each of which is incorporated by reference herein in its entirety.

“An immune cell engager” refers to one or more binding specificities that bind and/or activate an immune cell, e.g., a cell involved in an immune response. In embodiments, the immune cell is chosen from a T cell, an NK cell, a B cell, a dendritic cell, and/or the macrophage cell. The immune cell engager can be an antibody molecule, a receptor molecule (e.g., a full length receptor, receptor fragment, or fusion thereof (e.g., a receptor-Fc fusion)), or a ligand molecule (e.g., a full length ligand, ligand fragment, or fusion thereof (e.g., a ligand-Fc fusion)) that binds to the immune cell antigen (e.g., the NK cell antigen, the B cell antigen, the dendritic cell antigen, and/or the macrophage cell antigen). In embodiments, the immune cell engager specifically binds to the target immune cell, e.g., binds preferentially to the target immune cell. For example, when the immune cell engager is an antibody molecule, it binds to the immune cell antigen (e.g., the NK cell antigen, the B cell antigen, the dendritic cell antigen, and/or the macrophage cell antigen) with a dissociation constant of less than about 10 nM, and more typically, 10-100 pM.

As used herein, the term “specifically binding,” refers to the interaction between binding pairs (e.g., an antibody and an antigen, or a receptor and a ligand). In various instances, specifically binding can be embodied by an affinity constant of about 10-6 moles/liter, about 10-7 moles/liter, or about 10-8 moles/liter, or less.

The terms “equivalent” or “biological equivalent” are used interchangeably when referring to a particular molecule, biological, or cellular material and intend those having minimal homology while still maintaining desired structure or functionality. Non-limiting examples of equivalent polypeptides, include a polypeptide having at least 60%, or alternatively at least 65%, or alternatively at least 70%, or alternatively at least 75%, or alternatively 80%, or alternatively at least 85%, or alternatively at least 90%, or alternatively at least 95% identity thereto or for polypeptide sequences, or a polypeptide which is encoded by a polynucleotide or its complement that hybridizes under conditions of high stringency to a polynucleotide encoding such polypeptide sequences. Conditions of high stringency are described herein and incorporated herein by reference. Alternatively, an equivalent thereof is a polypeptide encoded by a polynucleotide or a complement thereto, having at least 70%, or alternatively at least 75%, or alternatively 80%, or alternatively at least 85%, or alternatively at least 90%, or alternatively at least 95% identity, or at least 97% sequence identity to the reference polynucleotide, e.g., the wild-type polynucleotide.

Non-limiting examples of equivalent polynucleotides having at least 60%, or alternatively at least 65%, or alternatively at least 70%, or alternatively at least 75%, or alternatively 80%, or alternatively at least 85%, or alternatively at least 90%, or alternatively at least 95%, or alternatively at least 97%, identity to a reference polynucleotide. An equivalent also intends a polynucleotide or its complement that hybridizes under conditions of high stringency to a reference polynucleotide.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. In certain embodiments, default parameters are used for alignment. A non-limiting exemplary alignment program is BLAST, using default parameters. In particular, exemplary programs include BLASTN and BLASTP, using the following default parameters: Genetic code=standard; filter-none; strand=both; cutoff=60; expect=10; Matrix=BLOSUM62; Descriptions=50 sequences; sort by=HIGH SCORE; Databases-non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. Sequence identity and percent identity can determined by incorporating them into clustalW (available at the web address: genome.jp/tools/clustalw/, last accessed on Jan. 13, 2017).

“Homology” or “identity” or “similarity” refers to sequence similarity between two peptides or between two nucleic acid molecules. Homology can be determined by comparing a position in each sequence that may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure. In some embodiments, BLAST (accessible at blast.ncbi.nlm.nih.gov/Blast.cgi) or Clustal Omega (accessible at www.ebi.ac.uk/Tools/msa/clustalo/) are used in determining the identity. In further embodiments, default setting is applied.

“Homology” or “identity” or “similarity” can also refer to two nucleic acid molecules that hybridize under stringent conditions.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

The terms “antigen” and “antigenic” refer to molecules with the capacity to be recognized by an antibody or otherwise act as a member of an antibody-ligand pair. “Specific binding” or “binding” refers to the interaction of an antigen with the variable regions of immunoglobulin heavy and light chains. Antibody-antigen binding may occur in vivo or in vitro. The skilled artisan will understand that macromolecules, including proteins, nucleic acids, fatty acids, lipids, lipopolysaccharides and polysaccharides have the potential to act as an antigen. The skilled artisan will further understand that nucleic acids encoding a protein with the potential to act as an antibody ligand necessarily encode an antigen. The artisan will further understand that antigens are not limited to full-length molecules, but can also include partial molecules. The term “antigenic” is an adjectival reference to molecules having the properties of an antigen. The term encompasses substances which are immunogenic, i.e., immunogens, as well as substances which induce immunological unresponsiveness, or anergy, i.e., anergens.

As used herein, a “tumor-specific antigen” and a “neoantigen” are used interchangeably and refer to an antigenic substance produced in tumor cells, capable of triggering an immune response in a subject. In some embodiments, such tumor-specific antigen is not expressed on or in a cell in the subject, which is not a cancer cell, or expressed at a lower level in a non-cancer cell compared to a cancer cell. In some embodiment, such tumor-specific antigen may still be expressed in or on some non-cancer cells. For example, a tumor-specific antigen may not be expressed on the cell surface of a non-cancer cell in the subject. In one embodiment, the tumor-specific antigen may be expressed in or on a non-cancer cell of the subject, but in a much lower level compared to a cancer cell. In another embodiment, the tumor-specific antigen may be expressed in or on a non-cancer cell of the subject which is not adjacent to a cancer or a cancer cell. Non-limiting examples of a tumor-specific antigen includes: Alphafetoprotein (AFP), Beta-2-microglobulin (B2M), Beta-human chorionic gonadotropin (Beta-hCG), Bladder Tumor Antigen (BTA), C-kit/CD117, CA15-3/CA27.29, CA19-9, CA-125, CA 27.29, Calcitonin, Carcinoembryonic antigen (CEA), Chromogranin A (CgA), Cytokeratin fragment 21-1, Des-gamma-carboxy prothrombin (DCP), Estrogen receptor (ER)/progesterone receptor (PR), Epithelial tumor antigen (ETA), Fibrin/fibrinogen, Gastrin, HE4, overexpressed HER2/neu, 5-HIAA, Lactate dehydrogenase, Melanoma-associated antigen (MAGE), MUC-1, Neuron-specific enolase (NSE), Nuclear matrix protein 22, Programmed death ligand 1 (PD-L1), Prostate-specific antigen (PSA), Prostatic Acid Phosphatase (PAP), Soluble mesothelin-related peptides (SMRP), Somatostatin receptor, Tyrosinase, Thyroglobulin, abnormal products of ras, p53, alpha folate receptor, 5T4, avB6 integrin, BCMA, B7-H3, B7-H6, CAIX, CD16, CD19, CD20, CD22, CD25, CD30, CD33, CD44, CD44v6, CD44v7/8, CD70, CD79a, CD79b, CD123, CD138, CD171, CEA, CSPG4, EGFR, EGFR family including ErbB2 (HER2), EGFRvni, EGP2, EGP40, EPCAM, EphA2, EpCAM, FAP, fetal AchR, FRoc, GD2, GD3, Glypican-3 (GPC3), HL A-A 1+M AGE 1, HLA-A2+MAGE1, HL A-A3+M AGE 1, HLA-AI+NY-ESO-1, HL A-A2+NY-ESO-1, HLA-A3+NY-ESO-1, IL-1 1Roc, IL-13Ra2, Lambda, Lewis-Y, Kappa, Mesothelin, Mucl, Mucl6, NCAM, NKG2D Ligands, NY-ESO-1, PRAME, PSCA, PSMA, ROR1, SSX, Survivin, TAG72, TEMs, VEGFR2, and WT-1.

In some embodiments, the neoantigen comprises, or alternatively consists essentially of, or yet further consists of one or more of: an embryonic antigen, an epithelial tumor antigen (ETA), melanoma-associated antigen (MAGE), or an abnormal product of ras. Additionally or alternatively, the cancer antigen is a human cancer antigen. In some embodiments, the cancer antigen comprises, or alternatively consists essentially of, or yet further consists of one or more of: alphafetoprotein (AFP, Genecards ID (GCID): GC04P073431, UniProtKB/Swiss-Prot: P02771), CA-125 (Mucin 16, Cell Surface Associated, Genecards ID (GCID): GC19M008848, UniProtKB/Swiss-Prot: Q8WX17), MUC-1 (Mucin 1, Cell Surface Associated, Genecards ID (GCID): GC01M155185, UniProtKB/Swiss-Prot: P15941), Tyrosinase (Genecards ID (GCID): GC11P089177, UniProtKB/Swiss-Prot: P14679), p53 (Tumor Protein P53, Genecards ID (GCID): GC17M007661, UniProtKB/Swiss-Prot: P04637), CD10 (Genecards ID (GCID): GC03P155024, UniProtKB/Swiss-Prot: P08473), CD19 (Genecards ID (GCID): GC16P032270, UniProtKB/Swiss-Prot: P15391), CD20 (Genecards ID (GCID): GC11P060475, UniProtKB/Swiss-Prot: P11836), CD21 (Genecards ID (GCID): GC01P207454, UniProtKB/Swiss-Prot: P20023), CD22 (Genecards ID (GCID): GC19P035319, UniProtKB/Swiss-Prot: P20273), CD25 (Genecards ID (GCID): GC10M006010, UniProtKB/Swiss-Prot: P01589), CD30 (Genecards ID (GCID): GC01P012063, UniProtKB/Swiss-Prot: P28908), CD33 (Genecards ID (GCID): GC19P051212, UniProtKB/Swiss-Prot: P20138), CD34 (Genecards ID (GCID): GC01M207880, UniProtKB/Swiss-Prot: P28906), CD37 (Genecards ID (GCID): GC19P049335, UniProtKB/Swiss-Prot: P11049), CD44v6 (an isoform containing the variant domain 6 of CD44 gene, Genecards ID (GCID): GC11P035139, UniProtKB/Swiss-Prot: P16070), CD45 (Genecards ID (GCID): GC01P198607, UniProtKB/Swiss-Prot: P08575), CDw52 (Genecards ID (GCID): GC01P026317, UniProtKB/Swiss-Prot: P31358), Fms-like tyrosine kinase 3 (FLT-3, CD135, Genecards ID (GCID): GC13M028003, UniProtKB/Swiss-Prot: P36888), c-Kit (CD117, Genecards ID (GCID): GC04P054657, UniProtKB/Swiss-Prot: P10721), CSF1R (Colony Stimulating Factor 1 Receptor, CD115, Genecards ID (GCID): GC05M150053, UniProtKB/Swiss-Prot: P07333), CD133 (Genecards ID (GCID): GC04M015965, UniProtKB/Swiss-Prot: 043490), PDGFR-α (Platelet Derived Growth Factor Receptor Alpha, CD140a, Genecards ID (GCID): GC04P054229, UniProtKB/Swiss-Prot: P16234), PDGFR-β (Platelet Derived Growth Factor Receptor Beta, CD 140b, Genecards ID (GCID): GC05M150113, UniProtKB/Swiss-Prot: P09619), chondroitin sulfate proteoglycan 4 (CSPG4, melanoma-associated chondroitin sulfate proteoglycan, Genecards ID (GCID): GC15M075674, UniProtKB/Swiss-Prot: Q6UVK1), EGFR (Epidermal Growth Factor Receptor, Genecards ID (GCID): GC07P055019, UniProtKB/Swiss-Prot: P00533), de2-7-EGFR (EGFRvIII, in which there is a deletion of the 801 bp encoded by exons 2-7, resulting in a truncated receptor (140 kDa) lacking 267 amino acid residues in the extracellular domain, with the insertion of a novel glycine residue at the site of the deletion. See, for example, Sugawa et al. Proc Natl Acad Sci USA. 1990 November; 87 (21): 8602-6 and Humphrey et al. Biochem Biophys Res Commun. 1991 Aug. 15; 178 (3): 1413-20), Folate binding protein (Genecards ID (GCID): GC11P072190, UniProtKB/Swiss-Prot: P15328), Her2neu (Genecards ID (GCID): GC17P039687, UniProtKB/Swiss-Prot: P04626), Her3 (Genecards ID (GCID): GC12P056376, UniProtKB/Swiss-Prot: P21860), PSMA (Prostate-Specific Membrane Antigen, Genecards ID (GCID): GC11M069030, UniProtKB/Swiss-Prot: Q04609), PSCA (Prostate Stem Cell Antigen, Genecards ID (GCID): GC08P142670, UniProtKB/Swiss-Prot: 043653), PSA (Prostate-Specific Antigen, Genecards ID (GCID): GC19P050854, UniProtKB/Swiss-Prot: P07288), Tumor-associated glycoprotein 72 (TAG-72, see, for example, Sheer et al. Cancer Res. 1988 Dec. 1; 48 (23): 6811-8), HLA-DR (Human Leukocyte Antigen-DR isotype, which is an αβ heterodimer. See, for example, Genecards ID (GCID): GC06P032439, UniProtKB/Swiss-Prot: P01903 for HLA Class II Histocompatibility Antigen, DR Alpha Chain; Genecards ID (GCID): GC06M032578, UniProtKB/Swiss-Prot: P01911 for HLA Class II Histocompatibility Antigen, DR-1 Beta Chain; Genecards ID (GCID): GC06Mn03715, UniProtKB/Swiss-Prot: P79483 for MHC Class II Antigen DR Beta 3 Chain; Genecards ID (GCID): GC06Mo03851, UniProtKB/Swiss-Prot: P13762 for HLA Class II Histocompatibility Antigen, DR Beta 4 Chain; and Genecards ID (GCID): GC06M046926, UniProtKB/Swiss-Prot: Q30154 for HLA Class II Histocompatibility Antigen, DR Beta 5 Chain), IGFR (Insulin Like Growth Factor 1 Receptor, Genecards ID (GCID): GC15P098648, UniProtKB/Swiss-Prot: P08069), IL3R (Interleukin 3 Receptor, see, for example, Genecards ID (GCID): GC0XP001336, UniProtKB/Swiss-Prot: P26951 for Interleukin 3 Receptor Subunit Alpha; and Genecards ID (GCID): GC22P036913, UniProtKB/Swiss-Prot: P32927 for Interleukin 3 Receptor Subunit Beta), fibroblast activating protein (FAP, Genecards ID (GCID): GC02M162170, UniProtKB/Swiss-Prot: Q12884), Carboanhydrase IX (MN/CA IX, Genecards ID (GCID): GC09P035673, UniProtKB/Swiss-Prot: Q16790), Carcinoembryonic antigen (CEA, Genecards ID (GCID): GC19P041709, UniProtKB/Swiss-Prot: P06731), EpCAM (Epithelial Cell Adhesion Molecule, Genecards ID (GCID): GC02P047345, UniProtKB/Swiss-Prot: P16422), CDCP1 (CUB Domain Containing Protein 1, Genecards ID (GCID): GC03M045082, UniProtKB/Swiss-Prot: Q9H5V8), DERL1 (Derlin1 or Derlin 1, Genecards ID (GCID): GC08M123013, UniProtKB/Swiss-Prot: Q9BUN8), Tenascin (for example, Genecards ID (GCID): GC06M046900, UniProtKB/Swiss-Prot: P22105 for Tenascin XB; Genecards ID (GCID): GC09M115019, UniProtKB/Swiss-Prot: P24821 for Tenascin C; Genecards ID (GCID): GC01M175291, UniProtKB/Swiss-Prot: Q92752 for Tenascin R; Genecards ID (GCID): GC01P175067, UniProtKB/Swiss-Prot: Q9UQP3 for Tenascin N; Genecards ID (GCID): GC06M046903, UniProtKB/Swiss-Prot: Q16473 for Tenascin XA), frizzled 1-10 (see, for example, Genecards ID (GCID): GC07P091264, UniProtKB/Swiss-Prot: Q9UP38 for frizzled 1 or FZD1; Genecards ID (GCID): GC17P044557, UniProtKB/Swiss-Prot: Q14332 for frizzled 2 or FZD2; Genecards ID (GCID): GC08P028494, UniProtKB/Swiss-Prot: Q9NPG1 for frizzled or FZD3; Genecards ID (GCID): GC11M086945, UniProtKB/Swiss-Prot: Q9ULV1 for frizzled 4 or FZD4; Genecards ID (GCID): GC02M207762, UniProtKB/Swiss-Prot: Q13467 for frizzled 5 or FZD5; Genecards ID (GCID): GC08P103298, UniProtKB/Swiss-Prot: 060353 for frizzled 6 or FZD6; Genecards ID (GCID): GC02P202034, UniProtKB/Swiss-Prot: 075084 for frizzled 7 or FZD7; Genecards ID (GCID): GC10M035638, UniProtKB/Swiss-Prot: Q9H461 for frizzled 8 or FZD8; Genecards ID (GCID):

GC07P073433, UniProtKB/Swiss-Prot: 000144 for frizzled 9 or FZD9; Genecards ID (GCID): GC12P130162, UniProtKB/Swiss-Prot: Q9ULW2 for frizzled 10 or FZD10), VEGFR2 (Vascular Endothelial Growth Factor Receptor 2, or KDR/FLK1, Genecards ID (GCID): GC04M055078, UniProtKB/Swiss-Prot: P35968), VEGFR3 (Vascular Endothelial Growth Factor Receptor 3, FLT4, or CD309, Genecards ID (GCID): GC05M180607, UniProtKB/Swiss-Prot: P35916), ENG (Endoglin, Genecards ID (GCID): GC09M127815, UniProtKB/Swiss-Prot: P17813), CLEC14 (see, for example, Genecards ID (GCID): GC14M038254, UniProtKB/Swiss-Prot: Q86T13 for C-Type Lectin Domain Containing 14A or CLEC14A), Tem1-8 (see, for example, Genecards ID (GCID): GC11M066314, UniProtKB/Swiss-Prot: Q9HCUO for Tumor Endothelial Marker 1 or TEM1; Genecards ID (GCID): GC22P035540, UniProtKB/Swiss-Prot: Q96D21 for Tumor Endothelial Marker 2 or TEM2; Genecards ID (GCID): GC17M039063, UniProtKB/Swiss-Prot: 8QIUK5 for Tumor Endothelial Marker 3, TEM3, Tumor Endothelial Marker 7, or TEM7; Genecards ID (GCID): GC11P073306, UniProtKB/Swiss-Prot: Q96PE2 for Tumor Endothelial Marker 4 or TEM4; Genecards ID (GCID): GC08P037785, UniProtKB/Swiss-Prot: Q96PE1 for Tumor Endothelial Marker 5 or TEM5; Genecards ID (GCID): GC07M047281, UniProtKB/Swiss-Prot: Q68CZ2 for Tumor Endothelial Marker 6 or TEM6; or Genecards ID (GCID): GC02P068977, UniProtKB/Swiss-Prot: Q9H6X2 for Tumor Endothelial Marker 8 or TEM8), or Tie2 (TEK Receptor Tyrosine Kinase, CD202b, Genecards ID (GCID): GC09P027109, UniProtKB/Swiss-Prot: Q02763). Each of the Genecard and UniProt webpages following the identified reference numbers is incorporated by references herein in its entirety.

As used herein, the term “embryonic antigen” refers to an antigen expressed by an embryonic cells. Some cancer antigens are embryonic antigens, whose expression is normally restricted to fetal cells but which are abnormally expressed in adult cells that have undergone malignant transformation. The gene encoding the embryonic antigen is silent in normal adult tissues but reactivated in cancer cells. An example of an embryonic antigen is carcinoembryonic antigen (CEA), which is normally expressed only in the liver, intestines and pancreas of the human fetus but is highly associated with colon, breast and ovarian cancers in the adult. Similarly, alpha fetoprotein (AFP) is normally expressed only in fetal liver and yolk sac, see, e.g., www.sciencedirect.com/topics/immunology-and-microbiology/alpha-fetoprotein. (AFP) is normally expressed only in fetal liver and yolk sac cells but is strongly linked to liver and testicular cancers in the adult. In some embodiments, the embryonic antigen comprises, or alternatively consists essentially of, or yet further consists of one or more of: Ephrin Type-A Receptor 2 (EphA2, Genecards ID (GCID): GC01M016124, UniProtKB/Swiss-Prot: P29317), Interleukin 13 Receptor Subunit Alpha 2 (IL 13Ra2, Genecards ID (GCID): GC0XM115003, UniProtKB/Swiss-Prot: Q14627) homodimers, carcinoembryonic antigen (CEA, Genecards ID (GCID): GC19P041709, UniProtKB/Swiss-Prot: P06731), or heat shock protein (hsp) gp96 (HSP96, Genecards ID (GCID): GC12P103930, UniProtKB/Swiss-Prot: P14625). Each of the Genecard and UniProt webpages following the identified reference numbers is incorporated by references herein in its entirety.

As used herein, the term “epithelial tumor antigen” or “ETA” refers to an antigen expression by an epithelial cancer cell. Non-limiting examples of ETA include Carcinoembryonic antigen (CEA, Genecards ID (GCID): GC19P041709, UniProtKB/Swiss-Prot: P06731), EpCAM (Genecards ID (GCID): GC02P047345, UniProtKB/Swiss-Prot: P16422), PSA (Prostate-Specific Antigen, Genecards ID (GCID): GC19P050854, UniProtKB/Swiss-Prot: P07288), HER-2/neu (Genecards ID (GCID): GC17P039687, UniProtKB/Swiss-Prot: P04626) and MUC1 (Genecards ID (GCID): GC01M155185, UniProtKB/Swiss-Prot: P15941). Each of the Genecard and UniProt webpages following the identified reference numbers is incorporated by references herein in its entirety.

As used herein, the term melanoma-associated antigen (MAGE) refers to a mammalian member of the MAGE (melanoma-associated antigen) gene family sharing a stretch of about 200 amino acids which has been named the MAGE conserved domain. Non-limiting examples of MAGE include, MAGE-B1 (Genecards ID (GCID): GC0XP030244, UniProtKB/Swiss-Prot: P43366), MAGEA1 (Genecards ID (GCID): GCOXP153179, UniProtKB/Swiss-Prot: P43355), MAGEA10 (Genecards ID (GCID): GCOXM152133, UniProtKB/Swiss-Prot: P43363), MAGEA11 (Genecards ID (GCID): GCOXP149688, UniProtKB/Swiss-Prot: P43364), MAGEA12 (Genecards ID (GCID): GCOXP152733, UniProtKB/Swiss-Prot: P43365), MAGEA2B (Genecards ID (GCID): GCOXP152714, UniProtKB/Swiss-Prot: P43356), MAGEA3 (Genecards ID (GCID): GCOXP152698, UniProtKB/Swiss-Prot: P43357), MAGEA4 (Genecards ID (GCID): GCOXP151912, UniProtKB/Swiss-Prot: P43358), MAGEA6 (Genecards ID (GCID): GC0XM152766, UniProtKB/Swiss-Prot: P43360), MAGEA8 (Genecards ID (GCID): GCOXP149881, UniProtKB/Swiss-Prot: P43361), MAGEA9 (Genecards ID (GCID): GCOXP149781, UniProtKB/Swiss-Prot: P43362), MAGEB1 (Genecards ID (GCID): GCOXP030244, UniProtKB/Swiss-Prot: P43366), MAGEB10 (Genecards ID (GCID): GC0XP027808, UniProtKB/Swiss-Prot: Q96LZ2), MAGEB 16 (Genecards ID (GCID): GC0XP035816, UniProtKB/Swiss-Prot: A2A368), MAGEB18 (Genecards ID (GCID): GC0XP026138, UniProtKB/Swiss-Prot: Q96M61), MAGEB2 (Genecards ID (GCID): GC0XP030215, UniProtKB/Swiss-Prot: 015479), MAGEB3 (Genecards ID (GCID): GC0XP030230, UniProtKB/Swiss-Prot: 015480), MAGEB4 (Genecards ID (GCID): GCOXP030241, UniProtKB/Swiss-Prot: 015481), MAGEB5 (Genecards ID (GCID): GC0XP026229, UniProtKB/Swiss-Prot: Q9BZ81), MAGEB6B (Genecards ID (GCID): GC0XP026192, UniProtKB/Swiss-Prot: Q8N7X4), MAGEC1 (Genecards ID (GCID): GCOXP141905, UniProtKB/Swiss-Prot: 060732), MAGEC2 (Genecards ID (GCID): GC0XM142202, UniProtKB/Swiss-Prot: Q9UBF1), MAGEC3 (Genecards ID (GCID): GCOXP141838, UniProtKB/Swiss-Prot: Q8TD91), MAGED1 (Genecards ID (GCID): GCOXP051803, UniProtKB/Swiss-Prot: Q9Y5V3), MAGED2 (Genecards ID (GCID): GC0XP054807, UniProtKB/Swiss-Prot: Q9UNF1), MAGED4 (Genecards ID (GCID): GCOXP052184, UniProtKB/Swiss-Prot: Q96JG8), MAGEE1 (Genecards ID (GCID): GC0XP076427, UniProtKB/Swiss-Prot: Q9HCI5), MAGEE2 (Genecards ID (GCID): GC0XM075782, UniProtKB/Swiss-Prot: Q8TD90), MAGEF1 (Genecards ID (GCID): GC03M184710, UniProtKB/Swiss-Prot: Q9HAY2), MAGEH1 (Genecards ID (GCID): GC0XP055452, UniProtKB/Swiss-Prot: Q9H213), MAGEL2 (Genecards ID (GCID): GC15M023643, UniProtKB/Swiss-Prot: Q9UJ55), NDN (Genecards ID (GCID): GC15M023686, UniProtKB/Swiss-Prot: Q99608), or NDNL2 (Genecards ID (GCID): GC15M029269, UniProtKB/Swiss-Prot: Q96MG7). Each of the Genecard and UniProt webpages following the identified reference numbers is incorporated by references herein in its entirety.

5-Fluorouracil (5-FU) belongs to the family of therapy drugs called pyrimidine based anti-metabolites. It is a pyrimidine analog, which is transformed into different cytotoxic metabolites that are then incorporated into DNA and RNA thereby inducing cell cycle arrest and apoptosis. Chemical equivalents are pyrimidine analogs which result in disruption of DNA replication. Chemical equivalents inhibit cell cycle progression at S phase resulting in the disruption of cell cycle and consequently apoptosis. Equivalents to 5-FU include prodrugs, analogs and derivative thereof such as 5′-deoxy-5-fluorouridine (doxifluoroidine), 1-tetrahydrofuranyl-5-fluorouracil (ftorafur), capecitabine (Xeloda®), S-1 (MBMS-247616, consisting of tegafur and two modulators, a 5-chloro-2,4-dihydroxypyridine and potassium oxonate), ralititrexed (tomudex), nolatrexed (Thymitaq, AG337), LY231514 and ZD9331, as described for example in Papamichael (1999) The Oncologist 4:478-487.

“5-FU based adjuvant therapy” refers to 5-FU alone or alternatively the combination of 5-FU with one or more other treatments, that include, but are not limited to radiation, methyl-CCNU, leucovorin, oxaliplatin (such as cisplatin), irinotecan, mitomycin, cytarabine, doxorubicin, cyclophosphamide, and levamisole, as well as an immunotherapy. Specific treatment adjuvant regimens are known in the art such as weekly Fluorouracil/Leucovorin, weekly Fluorouracil/Leucovorin+Bevacizumab, FOLFOX, FOLFOX-4, FOLFOX6, modified FOLFOX6 (mFOLFOX6), FOLFOX6 with bevacizumab, mFOLFOX6+Cetuximab, mFOLFOX6+Panitumumab, modified FOLFOX7 (mFOLFOX7), FOLFIRI, FOLFIRI with Bevacizumab, FOLFIRI+Ziv-aflibercept, FOLFIRI with Cetuximab, FOLFIRI+Panitumumab, FOLFIRI+Ramucirumab, FOLFOXIRI, FOLFIRI with FOLFOX6, FOLFOXIRI+Bevacizumab, FOLFOXIRI+Cetuximab, FOLFOXIRI+Panitumumab, Roswell Park Fluorouracil/Leucovorin, Roswell Park Fluorouracil/Leucovorin +Bevacizumab, Simplified Biweekly Infusional Fluorouracil/Leucovorin, Simplified Biweekly Infusional Fluorouracil/Leucovorin+Bevacizumab, and MOF (semustine(methyl-CCNU), vincrisine (Oncovin®) and 5-FU). For a review of these therapies see Beaven and Goldberg (2006) Oncology 20 (5): 461-470 as well as www.cancertherapyadvisor.com/home/cancer-topics/gastrointestinal-cancers/gastrointestinal-cancers-treatment-regimens/colon-cancer-treatment-regimens/. Other chemotherapeutics can be added, e.g., oxaliplatin or irinotecan.

Capecitabine is a prodrug of (5-FU) that is converted to its active form by the tumor-specific enzyme PynPase following a pathway of three enzymatic steps and two intermediary metabolites, 5′-deoxy-5-fluorocytidine (5′-DFCR) and 5′-deoxy-5-fluorouridine (5′-DFUR). Capecitabine is marketed by Roche under the trade name Xeloda®.

Leucovorin (Folinic acid) is an adjuvant used in cancer therapy. It is used in synergistic combination with 5-FU to improve efficacy of the chemotherapeutic agent. Without being bound by theory, addition of Leucovorin is believed to enhance efficacy of 5-FU by inhibiting thymidylate synthase. It has been used as an antidote to protect normal cells from high doses of the anticancer drug methotrexate and to increase the antitumor effects of fluorouracil (5-FU) and tegafur-uracil. It is also known as citrovorum factor and Wellcovorin. This compound has the chemical designation of L-Glutamic acid N-[4-[(2-amino-5-formyl-1,4,5,6,7,8-hexahydro-4-oxo-6-pteridinyl)methyl]amino] benzoyl], calcium salt (1:1).

“Oxaliplatin” (Eloxatin) is a platinum-based chemotherapy drug in the same family as cisplatin and carboplatin. It is typically administered in combination with fluorouracil and leucovorin in a combination known as FOLFOX for the treatment of colorectal cancer. Compared to cisplatin, the two amine groups are replaced by cyclohexyldiamine for improved antitumor activity. The chlorine ligands are replaced by the oxalato bidentate derived from oxalic acid in order to improve water solubility. Equivalents to Oxaliplatin are known in the art and include, but are not limited to cisplatin, carboplatin, aroplatin, lobaplatin, nedaplatin, and JM-216 (see Mckeage et al. (1997) J. Clin. Oncol. 201:1232-1237 and in general, Chemotherapy for Gynecological Neoplasm, Curr. Therapy and Novel Approaches, in the Series Basic and Clinical Oncology, Angioli et al. Eds., 2004).

“FOLFOX” is an abbreviation for a type of combination therapy that is used to treat cancer. This therapy includes leucovorin (“FOL”), 5-FU (“F”), and oxaliplatin (“OX”) and encompasses various regimens, such as FOLFOX-4, FOLFOX-6, modified FOLOX-6, and FOLFOX-7, which vary in doses and ways in which each of the three drugs are administered. “FOLFIRI” is an abbreviation for a type of combination therapy that is used treat cancer and comprises, or alternatively consists essentially of, or yet further consists of 5-FU, leucovorin, and irinotecan. Information regarding these treatments are available on the National Cancer Institute's web site, cancer.gov, last accessed on May 30, 2020 as well as www.cancertherapyadvisor.com/home/cancer-topics/gastrointestinal-cancers/gastrointestinal-cancers-treatment-regimens/colon-cancer-treatment-regimens/, last accessed on May 30, 2020.

Irinotecan (CPT-11) is sold under the trade name of Camptosar. It is a semi-synthetic analogue of the alkaloid camptothecin, which is activated by hydrolysis to SN-38 and targets topoisomerase I. Chemical equivalents are those that inhibit the interaction of topoisomerase I and DNA to form a catalytically active topoisomerase I-DNA complex. Chemical equivalents inhibit cell cycle progression at G2-M phase resulting in the disruption of cell proliferation.

The term “adjuvant” therapy refers to administration of a therapy or chemotherapeutic regimen to a patient in addition to the primary or initial treatment, such as after removal of a tumor by surgery. Adjuvant therapy is typically given to minimize or prevent a possible cancer reoccurrence. Alternatively, “neoadjuvant” therapy refers to administration of therapy or chemotherapeutic regimen before surgery, typically in an attempt to shrink the tumor prior to a surgical procedure to minimize the extent of tissue removed during the procedure. Additionally or alternatively, such adjuvant therapy potentials (i.e., sensitizes the subject to the original therapy) the subject may help reach one or more of clinical end points of the cancer treatment.

The phrase “first line” or “second line” or “third line” etc., refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as primary therapy and primary treatment.” See National Cancer Institute website at www.cancer.gov, last visited on May 1, 2008. Typically, a patient is given a subsequent chemotherapy regimen because the patient did not shown a positive clinical or sub-clinical response to the first line therapy or the first line therapy has stopped.

As used herein, the term “antifolate” intends a drug or biologic that impairs the function of folic acids, e.g., an antimetabolite agent that inhibits the use of a metabolite, i.e. another chemical that is part of normal metabolism. In cancer treatment, antimetabolites interfere with DNA production, thus cell division and growth of the tumor. Non-limiting examples of these agents are dihydrofolate reductase inhibitors, such as methotrexate, Aminopterin, and Pemetrexed; thymidylate synthase inhibitors, such as Raltitrexed or Pemetrexed; purine based, i.e. an adenosine deaminase inhibitor, such as Pentostatin, a thiopurine, such as Thioguanine and Mercaptopurine, a halogenated/ribonucleotide reductase inhibitor, such as Cladribine, Clofarabine, Fludarabine, or a guanine/guanosine: thiopurine, such as Thioguanine; or Pyrimidine based, i.e. cytosine/cytidine: hypomethylating agent, such as Azacitidine and Decitabine, a DNA polymerase inhibitor, such as Cytarabine, a ribonucleotide reductase inhibitor, such as Gemcitabine, or a thymine/thymidine: thymidylate synthase inhibitor, such as a Fluorouracil (5-FU).

In one aspect, the term “chemical equivalent” means the ability of the chemical to selectively interact with its target protein, DNA, RNA or fragment thereof as measured by the inactivation of the target protein, incorporation of the chemical into the DNA or RNA or other suitable methods. Chemical equivalents include, but are not limited to, those agents with the same or similar biological activity and include, without limitation a pharmaceutically acceptable salt or mixtures thereof that interact with and/or inactivate the same target protein, DNA, or RNA as the reference chemical.

The terms “oligonucleotide” or “polynucleotide” or “portion,” or “segment” thereof refer to a stretch of polynucleotide residues which is long enough to use in PCR or various hybridization procedures to identify or amplify identical or related parts of mRNA or DNA molecules. The polynucleotide compositions of this invention include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

As used herein, the term “microRNAs” or “miRNAs” refers to post-transcriptional regulators that typically bind to complementary sequences in the three prime untranslated regions (3′ UTRs) of target messenger RNA transcripts (mRNAs), usually resulting in gene silencing. Typically, miRNAs are short, non-coding ribonucleic acid (RNA) molecules, for example, 21 or 22 nucleotides long. The terms “microRNA” and “miRNA” are used interchangeably.

As used herein, the term “therapeutic agent” refers to an agent when used as a treatment having a beneficial or desired result.

As used herein, the term “splice-switching oligonucleotide” or “SSO” refers to a oligonucleotide comprising a modified nucleic acid, for example bridged nucleic acids (BNAs), base-pairing with pre-mRNA and disrupting normal splicing of transcripts by blocking the RNA-RNA base-pairing or protein-RNA binding interactions that occur between components of the splicing machinery and pre-mRNAs. In some embodiments, the SSO is a splice-switching antisense oligonucleotide comprising, or consisting essentially of, or yet further consisting of a backbone modified with 2′-O-methyl-phosphorothioate groups and approximately 60% BNAs, as well as two BNA-modified nucleotides at the 3′-end and one BNA-modified nucleotide at the 5′-end appear to work well for controlling and modulating the expression of specific exons, hence acting as antisense oligonucleotides enabling modulation and regulation of splicing events.

As used herein, the term “antisense polynucleotide” refers to a nucleic acid molecule that is complementary to at least a portion of a target nucleotide sequence of interest and hybridizes to the target nucleotide sequence under physiological conditions. Antisense molecules specifically hybridize with one or more nucleic acids encoding a preselected target nucleic acid. The terms target nucleic acid and nucleic acid encoding the target encompass DNA encoding the target, RNA (including pre-mRNA and mRNA) transcribed from such DNA, and also cDNA derived from such RNA. The hybridization of an antisense compound with its target nucleic acid interferes with the normal function of the nucleic acid. This modulation of function of a target nucleic acid by compounds which specifically hybridize to it is generally referred to as antisense. The functions of DNA to be interfered with include replication and transcription. The functions of RNA to be interfered with include all vital functions such, as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of the target.

Antisense polynucleotide design principles and protocols to follow are known in the art and described for example in Sazani, P., Kole, R., J. Clin. Invest. (2003), 112:481-486; Juliano, R., Alam, Md.R., Dixit, V., Kang, H., Nucleic Acids Res. (2008), 36:4158-4171; Chan, J. H., Lim, S., Wong, W. S., Clin. Exp. Pharmacol. Physiol. (2006), 33:533-540; Kurreck, J. Eur. J. Biochem. (2003), 270:1628-1644; and Crooke, S.T. Progress in antisense technology. Annu. Rev. Med. (2004), 55:61-95, all of which are incorporated herein in their entireties. Additional information is found in the Gene Link™ website (www.genelink.com/oligo_modifications_reference/OMR_mod_category_design.asp? mod_s p_cat_id=17 #: ˜: text=Anti-sense % 20Oligo %20Design %20Considerations-Selection % 20 of %20mRNA %20Target %20Site,chemical %20modifications %2C %20 is %20th en % 20designed %20around %20 that %20sequence, last accessed on Sep. 6, 2022).

As used herein, the term “oncogene” refers to a gene that has the potential to cause cancer. In certain tumor cells, oncogenes are expressed at high levels. Non-limiting examples of oncogenes include growth factors or mitogens such as c-Sis; receptor tyrosine kinases such as EGFR, HER2, PDGFR, and VEGFR; cytoplasmic tyrosine kinases such as Abl and kinases in the Src-family, Syk-ZAP-70 family, and BTK family of tyrosine kinases; cytoplasmic serine/threonine kinases and their regulatory subunits such as PIK3CA, PIK3R1, and RAF (e.g., RAF-1, A-RAF, B-RAF); regulatory GTPases such as RAS (e.g., KRAS); transcription factors such as MYC; or combinations thereof.

As used herein, the term “immune suppressor” refers to an agent reducing an immune response, such as PD-L1 expressed by a cancer cell.

As used herein, the term “an immune activator” refers to an agent inducing or enhancing an immune response, such as a cytokine as disclosed herein.

The term “immune checkpoint” refers to a component of the immune system which provides inhibitory signals to its components in order to regulate immune reactions. Known immune checkpoint proteins comprise CTLA-4, PD-1 and its ligands PD-L1 and PD-L2 and in addition LAG-3, BTLA, B7H3, B7H4, TIM3, KIR. The pathways involving LAGS, BTLA, B7H3, B7H4, TIM3, and KIR are recognized in the art to constitute immune checkpoint pathways similar to the CTLA-4 and PD-1 dependent pathways (see e.g. Pardon, 2012, Nature Rev Cancer 12:252-264; Mellman et al., 2011, Nature 480:480-489).

An “immune checkpoint inhibitor” refers to any agent inhibiting the function of an immune checkpoint protein. Inhibition includes reduction of function and full blockade. In particular the immune checkpoint protein is a human immune checkpoint protein. Thus the immune checkpoint protein inhibitor in particular is an inhibitor of a human immune checkpoint protein. In some embodiment, the immune checkpoint inhibitor comprises, or consists essentially of, or yet further consists of an antibody or an antigen binding fragment thereof specifically recognizing and binding the immune checkpoint protein.

The term “toxin” refers to a protein, enzyme, or polypeptide fragment thereof, or other molecule which is capable of arresting, retarding, or inhibiting the growth, division, multiplication or replication of a cell, such as a cancer cell, or which is capable of killing the cell. In some embodiments, the term “toxin” is intended to include, but not limited to, lytic proteins, bacteriocins (e.g., microcins and colicins), gyrase inhibitors, polymerase inhibitors, transcription inhibitors, translation inhibitors, DNases, and RNases.

As used herein, the term “purified” does not require absolute purity; rather, it is intended as a relative term. Thus, for example, a purified nucleic acid, peptide, protein, biological complexes, cell, virus or other active compound is one that is isolated in whole or in part from proteins or other contaminants. Generally, substantially purified peptides, proteins, biological complexes, cell, virus or other active compounds for use within the disclosure comprise more than 80% of all macromolecular species present in a preparation prior to admixture or formulation of the peptide, protein, biological complex, cell, virus or other active compound with a pharmaceutical carrier, excipient, buffer, absorption enhancing agent, stabilizer, preservative, adjuvant or other co-ingredient in a complete pharmaceutical formulation for therapeutic administration. More typically, the peptide, protein, biological complex, cell, virus or other active compound is purified to represent greater than 90%, often greater than 95% of all macromolecular species present in a purified preparation prior to admixture with other formulation ingredients. In other cases, the purified preparation may be essentially homogeneous, wherein other macromolecular species are not detectable by conventional techniques.

In some embodiments, the term “engineered” or “recombinant” refers to having at least one modification not normally found in a naturally occurring protein, polypeptide, polynucleotide, strain, wild-type strain or the parental host strain of the referenced species. In some embodiments, the term “engineered” or “recombinant” refers to being synthetized by human intervention.

The term “a regulatory sequence” “a regulatory element” “an expression control element” or “promoter” as used herein, intends a polynucleotide that is operatively linked to a polynucleotide to be transcribed and/or replicated, and facilitates the expression and/or replication of the polynucleotide. Non-limiting examples of a regulatory sequence include a promoter, an enhancer, or a polyadenylation sequence.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. Non-limiting examples of promoters include a cytomegalovirus CMV promoter or retroviral long terminal repeat (LTR) promoter. See, for example, Weber et al. Hum Gene Ther. 2007 September; 18 (9): 849-60.

An enhancer is a regulatory element that increases the expression of a target sequence. A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

The term “express” refers to the production of a gene product, such as mRNA, peptides, polypeptides or proteins. As used herein, “expression” refers to the process by which polynucleotides are transcribed into mRNA or the process by which the transcribed mRNA is subsequently being translated into peptides, polypeptides, or proteins. If the polynucleotide is derived from genomic DNA, expression may include splicing of the mRNA in a eukaryotic cell.

A “gene product” or alternatively a “gene expression product” refers to the amino acid (e.g., peptide or polypeptide) generated when a gene is transcribed and translated. In some embodiments, the gene product may refer to an mRNA or other RNA, such as an interfering RNA, generated when a gene is transcribed.

The term “encode” as it is applied to polynucleotides refers to a polynucleotide which is said to “encode” a polypeptide if, in its native state or when manipulated by methods well known to those skilled in the art, it can be transcribed to produce the mRNA for the polypeptide or a fragment thereof, and optionally translated to produce the polypeptide or a fragment thereof. The antisense strand is the complement of such a nucleic acid, and the encoding sequence can be deduced therefrom. Further, as used herein an amino acid sequence coding sequence refers to a nucleotide sequence encoding the amino acid sequence.

As used herein, the terms “overexpress,” “overexpression,” and the like are intended to encompass increasing the expression of a nucleic acid or a protein to a level greater than the exosome naturally contains. It is intended that the term encompass overexpression of endogenous, as well as heterologous nucleic acids and proteins.

As used herein, the term “homogeneous” in reference to a population of cell-derived vesicles refers to population of cell-derived vesicles that have a similar amount of an exogenous nucleic acid, a similar amount of an exogenous protein, are of a similar size, or combinations thereof. A homogenous population is one wherein about 65%, about 70%, about 75%, about 80%, about 85%, about 90%, about 95%, about 98%, or 100% of the cell-derived vesicles share at least one characteristic.

As used herein, the term “heterogeneous” in reference to a population of cell-derived vesicles refers to population of cell-derived vesicles that have differing amounts of an exogenous nucleic acid, differing amounts of an exogenous protein, are of a different size, or combinations thereof.

The term “substantially” refers to the complete or nearly complete extent or degree of a characteristic and in some aspects, defines the purity of the isolated or purified population of exosomes or microvesicle. For example, a substantially homogenous cell-derived vesicle population may be a cell-derived vesicle population that contains more than 60%, more than 70%, more than 80%, more than 90%, more than 95%, more than 98%, or 100% cell-derived vesicles that comprise at least one exogenous nucleic acid, protein, or both.

A “composition” is intended to mean a combination of active agent and another compound or composition, inert (for example, a detectable agent or label) or active, such as an adjuvant, diluent, binder, stabilizer, buffers, salts, lipophilic solvents, preservative, adjuvant or the like and include pharmaceutically acceptable carriers. A composition as disclosed herein can be a pharmaceutical composition. A “pharmaceutical composition” is intended to include the combination of an active agent with a carrier, inert or active, making the composition suitable for diagnostic or therapeutic use in vitro, in vivo or ex vivo. In some embodiments, a combination as used herein intends that the individual active ingredients of the compositions are separately formulated for use in combination, and can be separately packaged with or without specific dosages. The active ingredients of the combination can be administered concurrently or sequentially.

Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

A composition as disclosed herein can be a pharmaceutical composition. A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” refers to any of the standard pharmaceutical carriers such as sterile solutions, tablets, coated tablets, and capsules. Typically such carriers contain excipients such as starch, milk, sugar, certain types of clay, gelatin, stearic acids or salts thereof, magnesium or calcium stearate, talc, vegetable fats or oils, gums, glycols, or other known excipients. Such carriers may also include flavor and color additives or other ingredients. Examples of pharmaceutically acceptable carriers include, but are not limited to, the following: water, saline, buffers, inert, nontoxic solids (e.g., mannitol, talc). Compositions comprising such carriers are formulated by well-known conventional methods. Depending on the intended mode of administration and the intended use, the compositions may be in the form of solid, semi-solid, or liquid dosage forms, such, for example, as powders, granules, crystals, liquids, suspensions, liposomes, pastes, creams, salves, etc., and may be in unit-dosage forms suitable for administration of relatively precise dosages.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

A combination as used herein intends that the individual active ingredients of the compositions are separately formulated for use in combination, and can be separately packaged with or without specific dosages. The active ingredients of the combination can be administered concurrently or sequentially.

An “effective amount” intends an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present invention for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. In general, one will desire to administer an amount of the compound that is effective to achieve a serum level commensurate with the concentrations found to be effective in vitro. Determination of these parameters is well within the skill of the art. These considerations, as well as effective formulations and administration procedures are well known in the art and are described in standard textbooks.

“Therapeutically effective amount” of an agent refers to an amount of the agent that is an amount sufficient to obtain a pharmacological response; or alternatively, is an amount of the agent that, when administered to a patient with a specified disorder or disease, is sufficient to have the intended effect, e.g., treatment, alleviation, amelioration, palliation or elimination of one or more manifestations of the specified disorder or disease in the patient. A therapeutic effect does not necessarily occur by administration of one dose, and may occur only after administration of a series of doses. Thus, a therapeutically effective amount may be administered in one or more administrations.

As used herein, the term “vector” refers to a non-chromosomal nucleic acid comprising an intact replicon such that the vector may be replicated when placed within a cell, for example by a process of transformation. Vectors may be viral or non-viral. Viral vectors include retroviruses, lentiviruses, adenoviruses, herpesvirus, bacculoviruses, modified bacculoviruses, papovirus, or otherwise modified naturally occurring viruses. Exemplary non-viral vectors for delivering nucleic acid include naked DNA; DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethylene imine, in some cases contained in liposomes; and the use of ternary complexes comprising a virus and polylysine-DNA.

As used herein, the phrase “derived from” means isolated from, purified from, or engineered from, or any combination thereof.

A “viral vector” is defined as a recombinantly produced virus or viral particle that comprises a polynucleotide to be delivered into a cell, either in vivo, ex vivo or in vitro. Examples of viral vectors include retroviral vectors, lentiviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying, et al. (1999) Nat. Med. 5 (7): 823-827.

In aspects where modification of the cell is mediated by a lentiviral vector, a vector construct refers to the polynucleotide comprising the lentiviral genome or part thereof, and a therapeutic gene. As used herein, “transfection” or “transduction” in reference to delivery of exogenous nucleic acids carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. Retroviruses carry their genetic information in the form of RNA; however, once the virus infects a cell, the RNA is reverse-transcribed into the DNA form which integrates into the genomic DNA of the infected cell. The integrated DNA form is called a provirus. As used herein, lentiviral vector refers to a viral particle capable of introducing exogenous nucleic acid into a cell through a viral or viral-like entry mechanism. A “lentiviral vector” is a type of retroviral vector well-known in the art that has certain advantages in transducing nondividing cells as compared to other retroviral vectors. See, Trono D. (2002) Lentiviral vectors, New York: Spring-Verlag Berlin Heidelberg. In one aspect, the lentiviral vector is a non-replicating lentiviral vector.

Lentiviral vectors of this invention are based on or derived from oncoretroviruses (the sub-group of retroviruses containing MLV), and lentiviruses (the sub-group of retroviruses containing HIV). Examples include ASLV, SNV and RSV all of which have been split into packaging and vector components for lentiviral vector particle production systems. The lentiviral vector particle according to the invention may be based on a genetically or otherwise (e.g., by specific choice of packaging cell system) altered version of a particular retrovirus.

As used herein, the term “cytokine” refers to small proteins (about 5-20 kDa) important in cell signaling, including but not limited to chemokines, interferons, interleukins (ILs), lymphokines, and tumour necrosis factors, but generally not hormones. Cytokines are peptides and cannot cross the lipid bilayer of cells to enter the cytoplasm.

In some embodiments, a cytokine as used herein is an anti-cancer cytokine. As used herein, an anti-cancer cytokine refer to a cytokine suitable for use in treating a cancer optionally in combination with another cancer therapy. In some embodiments, the anti-cancer cytokine comprises, or alternatively consists essentially of, or yet further consists of any one or more of: interleukin 2 (IL-2), Interleukin 5 (IL-5), interleukin 12 (IL-12), interleukin 15 (IL-15), Interleukin 18 (IL-18), interleukin 21 (IL-21), interleukin 27 (IL-27), Interferon α (IFNα), Type I interferon (IFNI), Type II interferon (IFNII), Interferon γ (IFNγ), Interferon β (IFNβ), CD40 Ligand (CD40L), Colony Stimulating Factor 2 (CSF2), RANTES, Tumor Necrosis Factor α (TNFα), C—X—C Motif Chemokine Ligand 9 (CXCL9), C—X—C Motif Chemokine Ligand 10 (CXCL10), or C—X—C Motif Chemokine Ligand 11 (CXCL11).

Interleukin-2 (IL-2) is an interleukin, a type of cytokine signaling molecule in the immune system. It is a 15.5-16 kDa protein that regulates the activities of white blood cells (leukocytes, often lymphocytes) that are responsible for immunity. In some embodiments, the IL-2 is a human IL-2. In some embodiments, the IL-2 is of other species, such as a chimpanzee IL-2 having an NCBI Reference Sequence of XP_517425.1. Non-limiting exemplary sequences of this protein or the underlying gene can be found under Gene Cards ID: GC04M122451, HGNC (6001), NCBI Entrez Gene (3558), Ensembl (ENSG00000109471), OMIM® (147680), UniProtKB/Swiss-Prot (P60568), and Open Targets platform (ENSG00000109471), each of which is incorporated by reference herein in its entirety.

Interleukin 5 (IL-5) is an interleukin (IL) produced by type-2 T helper cells and mast cells. It acts as a growth and differentiation factor for both B cells and eosinophils; plays a major role in the regulation of eosinophil formation, maturation, recruitment and survival; and functions by binding to its receptor, which is a heterodimer, whose beta subunit is shared with the receptors for interleukin 3 (IL3) and colony stimulating factor 2 (CSF2/GM-CSF). The increased production of this cytokine may be related to pathogenesis of eosinophil-dependent inflammatory diseases. In some embodiments, the IL-5 is a human IL-5. Non-limiting exemplary sequences of this protein or the underlying gene can be found under Gene Cards ID: C05M132541, HGNC: 6016, NCBI Entrez Gene: 3567, Ensembl: ENSG00000113525, OMIM®: 147850, or UniProtKB/Swiss-Prot: P05113, each of which is incorporated by reference herein in its entirety.

Interleukin 12 (IL-12) is an interleukin that is naturally produced by dendritic cells, macrophages, neutrophils, and human B-lymphoblastoid cells (NC-37) in response to antigenic stimulation. In some embodiments, the IL-12 is a human IL-12. It is a heterodimeric cytokine comprising, or alternatively consisting essentially of, or yet further consisting of IL-12A (p35) and IL-12B (p40). Non-limiting exemplary sequences of IL 12A or the underlying gene can be found under Gene Cards ID: GC03P159988, GNC: 5969, NCBI Entrez Gene: 3592, Ensembl: ENSG00000168811, OMIM®: 161560, or UniProtKB/Swiss-Prot: P29459, each of which is incorporated by reference herein in its entirety. Non-limiting exemplary sequences of IL 12B or the underlying gene can be found under Gene Cards ID: GC05M159314, HGNC: 5970, NCBI Entrez Gene: 3593, Ensembl: ENSG00000113302, OMIM®: 161561, or UniProtKB/Swiss-Prot: P29460, each of which is incorporated by reference herein in its entirety.

Interleukin-15 (IL-15) is a cytokine with structural similarity to Interleukin-2 (IL-2). Like IL-2, IL-15 binds to and signals through a complex composed of IL-2/IL-15 receptor beta chain (CD122) and the common gamma chain (gamma-C, CD132). IL-15 is secreted by mononuclear phagocytes (and some other cells) following infection by virus(es). This cytokine induces the proliferation of natural killer cells. In some embodiments, the IL-15 is a human IL-15. Non-limiting exemplary sequences of this protein or the underlying gene can be found under Gene Cards ID: GC04P141636, HGNC: 5977, NCBI Entrez Gene: 3600, Ensembl: ENSG00000164136, OMIM®: 600554, or UniProtKB/Swiss-Prot: P40933, each of which is incorporated by reference herein in its entirety.

Interleukin 18 (IL-18) is a proinflammatory cytokine. Many cell types, both hematopoietic cells and non-hematopoietic cells, have the potential to produce IL-18. IL-18 can modulate both innate and adaptive immunity and its dysregulation can cause autoimmune or inflammatory diseases. In some embodiments, the IL-18 is a human IL-18. Non-limiting exemplary sequences of this protein or the underlying gene can be found under Gene Cards ID: GC11M112143, HGNC: 5986, NCBI Entrez Gene: 3606, Ensembl: ENSG00000150782, OMIM®: 600953, UniProtKB/Swiss-Prot: Q14116, each of which is incorporated by reference herein in its entirety.

C—X—C Motif Chemokine Ligand 10 (CXCL10) involves in NK cell/T cell recruitment. It directs the activity of the effective CD8 and CD4 T cells and is an antiangiogenic/anti-tumor protein. In some embodiments, at the tumor site or inflammation site, CSCL10 is released, binds to CXCR3 on CD8 positive T cells resulting in release of granzyme B and IFNγ, and binds to CXCR3 on CD4 positive T cells resulting in release of IFNγ. See, for example, Bagheri et al. Cell Oncol (Dordr). 2020 June; 43 (3): 353-365. In some embodiments, the CXCL 10 is a human CXCL10. Non-limiting exemplary sequences of this protein or the underlying gene can be found under Gene Cards ID: GC04M076021, HGNC: 10637, NCBI Entrez Gene: 3627, Ensembl: ENSG00000169245, OMIM®: 147310, or UniProtKB/Swiss-Prot: P02778, each of which is incorporated by reference herein in its entirety.

Interleukin-21 (IL-21) is a cytokine that has potent regulatory effects on cells of the immune system, including natural killer (NK) cells and cytotoxic T cells that can destroy virally infected or cancerous cells. This cytokine induces cell division/proliferation in its target cells. IL-21 signaling and biological effects on target cells include, but are not limited to, B cell proliferation, immunoglobulin production, T follicular helper (Tfh) cell proliferation, cytotoxic T cell (CTL) proliferation and anti-tumor activity, NK proliferation and antibody-dependent cellular cytotoxicity (ADCC) activity, and suppression of regulatory T cells (Tregs) proliferation. More details can be found in Croce M, et al. J Immunol Res. 2015; 2015:696578. For example, IL-21 released by CD4 T cells binds to a complex formed by IL-21R, γc, JAK-1 and JAK-3. By phosphorylating STAT-5, STAT-3 or STAT-1, various IL-21 target genes are expressed, such as GzmA, GzmB, IL-10, Bim, Socs, Bxl-6, Jak3 etc. Accordingly, certain immune enhancing effects are achieved, including, but not limited to, for B cells: cell proliferation, immunoglobulin production, plasma cell differentiation; for Tfh cells, cell differentiation and proliferation; for CTL, cell proliferation, survival, anti-tumor activity, CD28 and L-selectin expression; for NK cells: proliferation, antitumor activity, ADCC activity; for Th17 cells: differentiation, proliferation IL-23R expression; and for Treg: inhibiting survival, inhibiting generation. Additionally, certain immune regulatory effects are also achieved, including, but not limited to, for T regulatory type 1 (Tr1) cells: differentiation, proliferation, IL-10 production; for dendritic cells (DCs): apoptosis, inhibition of antigen-presenting cell (APC) APC function; and for regulatory B cells (Breg/B10): differentiation, proliferation and IL-10 production. In some embodiments, the IL-21 is a human IL-21. Non-limiting exemplary sequences of this protein or the underlying gene can be found under Gene Cards ID: GC04M122612, HGNC: 6005, NCBI Entrez Gene: 59067, Ensembl: ENSG00000138684, OMIM®: 605384, and UniProtKB/Swiss-Prot: Q9HBE4, each of which is incorporated by reference herein in its entirety.

Interleukin 27 (IL-27) is a member of the IL-12 cytokine family. It is a heterodimeric cytokine that is composed of two distinct genes, Epstein-Barr virus-induced gene 3 (EBI3) and IL-27p28. IL-27 is expressed by antigen presenting cells and interacts with a specific cell-surface receptor complex known as IL-27 receptor (IL-27R). IL-27 induces differentiation of the diverse populations of T cells in the immune system and also upregulates IL-10. In some embodiments, the IL-27 is a human IL-27. Non-limiting exemplary sequences of this protein or the underlying gene can be found under Gene Cards ID: GC16M028514, HGNC: 19157, NCBI Entrez Gene: 246778, Ensembl: ENSG00000197272, OMIM®: 608273, or UniProtKB/Swiss-Prot: Q8NEV9, each of which is incorporated by reference herein in its entirety.

Unbiased gene expression analysis was performed on tumor patient treated with G207, an attenuated replication-defective HSV-1 vector that expresses β-galactosidase following infection. The results showed that patients treated with G207 in clinical trial who had high IL-27 levels in their treated tumors also lived longer, indicating there was a direct correlation between survival and IL-27 levels. Accordingly, IL-27 is used as a therapeutic agent to be delivered as described herein.

Interferon α (IFNα) is produced mainly by plasmacytoid dendritic cells (pDCs). They are mainly involved in innate immunity against viral infection. The genes responsible for their synthesis come in 13 subtypes that are called IFNA1 (Genecards ID (GCID): GC09P021522, UniProtKB/Swiss-Prot: P01562), IFNA2 (GCID: GC09M021384, niProtKB/Swiss-Prot: P01563), IFNA4 (GCID: GC09M021186, UniProtKB/Swiss-Prot: P05014), IFNA5 (GCID: GC09M021304, UniProtKB/Swiss-Prot: P01569), IFNA6 (GCID: GC09M021349, UniProtKB/Swiss-Prot: P05013), IFNA7 (GCID: GC09M021473, UniProtKB/Swiss-Prot: P01567), IFNA8 (GCID: GC09P021409, UniProtKB/Swiss-Prot: P32881), IFNA10 (GCID: GC09M021474, UniProtKB/Swiss-Prot: P01566), IFNA13 (GCID: GC09M021367, UniProtKB/Swiss-Prot: P01562), IFNA14 (GCID: GC09M021239, UniProtKB/Swiss-Prot: P01570), IFNA16 (GCID: GC09M021216, UniProtKB/Swiss-Prot: P05015), IFNA17 (GCID: GC09M021227, UniProtKB/Swiss-Prot: P01571), and IFNA21 (GCID: GC09M021165, UniProtKB/Swiss-Prot: P01568). These genes are found together in a cluster on chromosome 9. In some embodiments, the IFNα is a human IFNα. Each of the Genecard and UniProt webpages following the identified reference numbers is incorporated by references herein in its entirety.

The term “type I interferon” or “IFNI” as used herein is intended to refer to members of the type I interferon family of molecules that that binds to a cell surface receptor complex known as the IFN-α receptor (IFNAR) that consists of IFNAR1 and IFNAR2 chains. Non-limiting examples of type I interferon include IFN-α (such as IFN-αl (Genecards ID (GCID): GC09P021522, UniProtKB/Swiss-Prot: P01562), IFN-α2 (GCID: GC09M021384, niProtKB/Swiss-Prot: P01563), IFN-α4 (GCID: GC09M021186, UniProtKB/Swiss-Prot: P05014), IFN-α5 (GCID: GC09M021304, UniProtKB/Swiss-Prot: P01569), IFN-α6 (GCID: GC09M021349, UniProtKB/Swiss-Prot: P05013), IFN-α7 (GCID: GC09M021473, UniProtKB/Swiss-Prot: P01567), IFN-α8 (GCID: GC09P021409, UniProtKB/Swiss-Prot: P32881), IFN-α10 (GCID: GC09M021474, UniProtKB/Swiss-Prot: P01566), IFN-α13 (GCID: GC09M021367, UniProtKB/Swiss-Prot: P01562), IFN-α14 (GCID: GC09M021239, UniProtKB/Swiss-Prot: P01570), IFN-α16 (GCID: GC09M021216, UniProtKB/Swiss-Prot: P05015), IFN-α17 (GCID: GC09M021227, UniProtKB/Swiss-Prot: P01571), and IFN-α21 (GCID: GC09M021165, UniProtKB/Swiss-Prot: P01568)), IFN-β (such as IFN-β1 (GCID: GC09M021077, UniProtKB/Swiss-Prot: P01574) or IFN-β3 (UniProtKB/Swiss-Prot: T2K1E0)), IFN-κ (GCID: GC09P027514, UniProtKB/Swiss-Prot: Q9POWO), IFN-δ, IFN-ω (GCID: GC09M021480, UniProtKB/Swiss-Prot: Q86WN2), IFN-τ, IFN-ω (GCID: GC09M021140, UniProtKB/Swiss-Prot: P05000), and IFN-ζ. In some embodiments, the type I interferon is a human type I interferon. Each of the Genecard and UniProt webpages following the identified reference numbers is incorporated by references herein in its entirety.

The term “type II interferon” denotes interferons that bind to the interferon-gamma receptor (IFNGR). The type II interferons present in humans comprise interferon γ. Interferon γ(IFNγ) is a soluble cytokine that is a member of the type II interferon class. It is secreted by cells of both the innate and adaptive immune systems. The active protein is a homodimer that binds to the interferon gamma receptor which triggers a cellular response to viral and microbial infections. In some embodiments, the IFNγ is a human IFNγ. Non-limiting exemplary sequences of this protein or the underlying gene can be found under Gene Cards ID: GC12M068154, HGNC: 5438, NCBI Entrez Gene: 3458, Ensembl: ENSG00000111537, OMIM®: 147570, or UniProtKB/Swiss-Prot: P01579, each of which is incorporated by reference herein in its entirety.

Interferon β (IFNβ) is a cytokine that belongs to the interferon family of signaling proteins, which are released as part of the innate immune response to pathogens, and has antiviral, antibacterial, and anticancer properties. In some embodiments, the IFNβ is a human IFNβ. Non-limiting exemplary sequences of this protein or the underlying gene can be found under Gene Cards ID: GC09M021077, HGNC: 5434, NCBI Entrez Gene: 3456, Ensembl: ENSG00000171855, OMIM®: 147640, or UniProtKB/Swiss-Prot: P01574, each of which is incorporated by reference herein in its entirety.

CD40 Ligand (CD40L) is a protein that is primarily expressed on activated T cells and is a member of the TNF superfamily of molecules. It binds to CD40 (protein) on antigen-presenting cells (APC), which leads to many effects depending on the target cell type. In total CD40L has three binding partners: CD40, α5β1 integrin and αIIbβ3. CD154 acts as a costimulatory molecule and is particularly important on a subset of T cells called T follicular helper cells (TFH cells). On THE cells, CD154 promotes B cell maturation and function by engaging CD40 on the B cell surface and therefore facilitating cell-cell communication. In some embodiments, the CD40L is a human CD40L. Non-limiting exemplary sequences of this protein or the underlying gene can be found under Gene Cards ID: GCOXP136649, HGNC: 11935, NCBI Entrez Gene: 959, Ensembl: ENSG00000102245, OMIM®: 300386, or UniProtKB/Swiss-Prot: P29965, each of which is incorporated by reference herein in its entirety.

Colony Stimulating Factor 2 (CSF2) is a monomeric glycoprotein secreted by macrophages, T cells, mast cells, natural killer cells, endothelial cells and fibroblasts that functions as a cytokine stimulating stem cells to produce granulocytes (neutrophils, eosinophils, and basophils) and monocytes. In some embodiments, the CSF2 is a human CSF2. Non-limiting exemplary sequences of this protein or the underlying gene can be found under Gene Cards ID: GC05P132073, HGNC: 2434, NCBI Entrez Gene: 1437, Ensembl: ENSG00000164400, OMIM®: 138960, or UniProtKB/Swiss-Prot: P04141, each of which is incorporated by reference herein in its entirety.

RANTES is also referred to as C—C Motif Chemokine Ligand 5 or CCL5. It is an 8 kDa protein classified as a chemotactic cytokine or chemokine. CCL5 is chemotactic for T cells, eosinophils, and basophils, and plays an active role in recruiting leukocytes into inflammatory sites. With the help of particular cytokines (i.e., IL-2 and IFN-γ) that are released by T cells, CCL5 also induces the proliferation and activation of certain natural-killer (NK) cells to form CHAK (CC-Chemokine-activated killer) cells. It is also an HIV-suppressive factor released from CD8+ T cells. In some embodiments, the RANTES is a human RANTES. Non-limiting exemplary sequences of this protein or the underlying gene can be found under Gene Cards ID: GC17M035871, GNC: 10632, NCBI Entrez Gene: 6352, Ensembl: ENSG00000271503, OMIM®: 187011, or UniProtKB/Swiss-Prot: P13501, each of which is incorporated by reference herein in its entirety.

Tumor Necrosis Factor α (TNFα, or TNF) is a cytokine released by macrophages upon detection of an infection to alert other immune system cells as part of an inflammatory response. TNF is a member of the TNF superfamily, which consists of various transmembrane proteins with a homologous TNF domain. In some embodiments, the TNFα is a human TNFα. Non-limiting exemplary sequences of this protein or the underlying gene can be found under Gene Cards ID: GC06P055202, HGNC: 11892, NCBI Entrez Gene: 7124, Ensembl: ENSG00000232810, OMIM®: 191160, or UniProtKB/Swiss-Prot: P01375, each of which is incorporated by reference herein in its entirety.

C—X—C Motif Chemokine Ligand 9 (CXCL9) is a small cytokine belonging to the CXC chemokine family that is also known as monokine induced by gamma interferon (MIG). The CXCL9 is one of the chemokine which plays role to induce chemotaxis, promote differentiation and multiplication of leukocytes, and cause tissue extravasation. The CXCL9/CXCR3 receptor regulates immune cell migration, differentiation, and activation. Immune reactivity occurs through recruitment of immune cells, such as cytotoxic lymphocytes (CTLs), natural killer (NK) cells, NKT cells, and macrophages. In some embodiments, the CXCL9 is a human CXCL9. Non-limiting exemplary sequences of this protein or the underlying gene can be found under Gene Cards ID: GC04M076001, HGNC: 7098, NCBI Entrez Gene: 4283, Ensembl: ENSG00000138755, OMIM®: 601704, or UniProtKB/Swiss-Prot: Q07325, each of which is incorporated by reference herein in its entirety.

C—X—C Motif Chemokine Ligand 11 (CXCL11) is a small cytokine belonging to the CXC chemokine family that is also called Interferon-inducible T-cell alpha chemoattractant (I-TAC) and Interferon-gamma-inducible protein 9 (IP-9). It is highly expressed in peripheral blood leukocytes, pancreas and liver, with moderate levels in thymus, spleen and lung and low expression levels were in small intestine, placenta and prostate. In some embodiments, the CXCL11 is a human CXCL11. Non-limiting exemplary sequences of this protein or the underlying gene can be found under Gene Cards ID: GCID: GC04M076033, HGNC: 10638, NCBI Entrez Gene: 6373, Ensembl: ENSG00000169248, OMIM®: 604852, or UniProtKB/Swiss-Prot: 014625, each of which is incorporated by reference herein in its entirety.

As used herein, the term “detectable marker” refers to at least one marker capable of directly or indirectly, producing a detectable signal. A non-exhaustive list of this marker includes enzymes which produce a detectable signal, for example by colorimetry, fluorescence, luminescence, such as horseradish peroxidase, alkaline phosphatase, β-galactosidase, glucose6 phosphate dehydrogenase, chromophores such as fluorescent, luminescent dyes, groups with electron density detected by electron microscopy or by their electrical property such as conductivity, amperometry, voltammetry, impedance, detectable groups, for example whose molecules are of sufficient size to induce detectable modifications in their physical and/or chemical properties, such detection may be accomplished by optical methods such as diffraction, surface plasmon resonance, surface variation, the contact angle change or physical methods such as atomic force spectroscopy, tunnel effect, or radioactive molecules such as ³²P, ³⁵S, ⁸⁹Zr or ¹²⁵I.

### Modes For Carrying Out The Disclosure

The instant disclosure provides methods for making therapeutic EVs. The instant disclosure also provides methods for delivering a therapeutic agent to a cancer cell using cancer cell-produced EVs. In addition, the instant disclosure provides methods for expressing a therapeutic agent in a cancer cell. In some embodiments, the therapeutic agent is a polypeptide expressed from an exogenous polynucleotide, wherein the exogenous polynucleotide was delivered to the cancer cell by an EV. The disclosure further shows that chemokine and/or cytokine-loaded EVs isolated from cancer or tumor cells provide an effective cancer therapy platform and enables precise immunotherapy delivery to target tumor cells. The EVs of the instant disclosure have the surprising ability to cross the blood brain barrier and to traffic between tumors without specifically targeting non-tumor cells, tissues or organs. The EVs of the instant disclosure can also increase immune cell infiltration to a tumor site, and stimulate anti-cancer immune response. The EVs of the instant disclosure can specifically target tumor cells both when delivered systemically and when delivered locally. In some embodiments, the EVs of the instant disclosure propagate and traffic between tumor cells. Thus, as disclosed below, provided herein are in vitro and in vivo methods for achieving these therapeutic results.

### Cell-derived Vesicles

Cell-derived vesicles, also referred to as extracellular vesicles (EVs), are membrane surrounded structures that are released by cells in vitro and in vivo. Extracellular vesicles can contain proteins, lipids, and nucleic acids and can mediate intercellular communication between different cells, including different cell types, in the body. Two types of extracellular vesicles are exosomes and microvesicles. Exosomes are small lipid-bound, cellularly secreted vesicles that mediate intercellular communication via cell-to-cell transport of proteins and RNA (El Andaloussi, S. et al. (2013) Nature Reviews: Drug Discovery 12 (5): 347-357). Exosomes range in size from approximately 30 nm to about 200 nm. Exosomes are released from a cell by fusion of multivesicular endosomes (MVE) with the plasma membrane. Microvesicles, on the other hand, are released from a cell upon direct budding from the plasma membrane (PM). Microvesicles are typically larger than exosomes and range from approximately 100 nm to 1 μm.

Exosomes are extracellular vesicles generated by all cells and they carry nucleic acids, proteins, lipids, and metabolites. They are mediators of near and long-distance intercellular communication in health and disease and affect various aspects of cell biology. Hallmarks of exosomes include, but are not limited to, CD9, CD63, CD81, certain transmembrane proteins, cholesterol, flotillin-1, tumor susceptibility gene 101 protein (TSG101), heat shock 70 kDa protein (HSP70), heat shock 90 kDa protein (HSP90), and ALG-2 interacting protein X (ALIX). Exosomes also provide a cell-to-cell transit system in the human body with pleiotropic functions, such as regulation of gene transcription and translation, survival and proliferation, reproduction and development, angiogenesis and wound healing, waste management, host-microbiome interaction and viral immunity, balance of immune response and regulation of central and peripheral immunity, receptor-ligand signaling, apoptosis, cellular differentiation and neoplasia, cellular migration and metastatic disease, and metabolic reprogramming and regulation. See, for example, Kalluri and LeBleu. Science. 2020 Feb. 7; 367 (6478): eaau6977.

Extracellular vesicle (EV) is a membrane vesicle secreted by most cell types and is 40-160 nm in size. EVs are end-product of the endocytic recycling pathway. Like viruses, EVs are forms of horizontal genetic transfer. Some viruses, for example, lentivirus or retrovirus, hijack this pathway during the envelopment process. See, for example, www.biolegend.com/en-us/exosomes and Chicón-Bosch and Tirado. Cells. 2020 Jan. 17; 9 (1): 241.

EVs has been used as therapeutics, such as by direct EV modification comprising plasmatic exosome isolation and ex vivo exosome manipulation (e.g., small molecule drug loading, or therapeutic RNAs or miRNAs loading); indirect EV engineering, such as induced Dendritic Cells (iDC) isolation or mesenchymal stem cells (MSCs) isolation, parental cell engineering and engineered exosome isolation; or reinfusion in patient. See, for example, Campanella C, et al. Int J Mol Sci. 2019 Jan. 9; 20 (2): 236.

In one aspect, provided herein is an isolated or purified extracellular vesicle (EV) isolated or purified from a cancer cell, wherein the EV comprises a therapeutic agent. In another aspect, the EV further comprises a surface membrane associated protein that selectively targets a cancer cell. The cells can be primary cancer cells or cultured cancer cells. The cells can be commercially available cancer cells, such as those that can be obtained from the American Type Culture Collection (ATCC). Examples are provided herein.

In one aspect, provided is an extracellular vesicle (EV) isolated from a cancer cell as described herein. In some embodiments, the cancer cell is a cancer of the central nervous system. In some embodiments, the cancer of the central nervous system is a glioblastoma or a glioma. In some embodiments, the EV further comprises a therapeutic agent.

In some embodiments, the EVs further comprise a surface membrane associated protein that selectively targets a cancer cell.

In some embodiments, the cancer cells are engineered to produce the therapeutic agent in the EVs.

In some embodiments, the therapeutic agent comprises, or consists essentially of, or yet further consists of a polypeptide (a “therapeutic polypeptide”). In some embodiments, the cancer cells are engineered to express the polypeptide. In some embodiments, the cancer cells are transfected with an exogenous polynucleotide (e.g. a gene expression cassette, or an expression vector) that encodes the polypeptide. In some embodiments, the cancer cells load the polypeptide into the EVs and secrete the loaded EVs, which can be isolated and used for the methods of the instant disclosure. In some embodiments, the polypeptide is a cytokine or a toxin. In some embodiments, the polypeptide is an antibody, or a fragment of an antibody. In some embodiments, the polypeptide is a secreted antibody.

In some embodiments, the EVs are loaded with the therapeutic agent by sonicating the EVs together with the therapeutic agent.

In some embodiments, the therapeutic agent is a virus and the EVs are loaded with the virus by sonicating the EVs together with the virus.

In some embodiments, the therapeutic agent comprises, or consists essentially of, or yet further consists of a therapeutic polynucleotide. In some embodiments, the EVs are loaded with the therapeutic polynucleotide by sonicating the EVs together with the therapeutic polynucleotide.

In some embodiments, the therapeutic polynucleotide comprises, or consists essentially of, or yet further consists of, or expresses a splice-switching oligonucleotide (SSO) or an antisense polynucleotide. Additionally or alternatively, the SSO or antisense polynucleotide reduces the expression of an oncogene or an immune suppressor in a cell of the cancer to be treated. In a specific embodiment, the antisense polynucleotide is an anti-PDL1 antisense oligonucleotide.

In some embodiments, the oncogene comprises, or consists essentially of, or yet further consists of one or more of: Insulin Like Growth Factor 1 Receptor (IGFR), Tumor Protein P53 (p53), or MDM2 Proto-Oncogene (MDM2).

In some embodiments, the immune suppressor comprises, or consists essentially of, or yet further consists of PD-L1.

In some embodiments, the therapeutic polynucleotide expresses a protein toxin or an immune activator.

In some embodiments, the EV further comprises a vector that comprises, or consists essentially of, or yet further consists of the polynucleotide.

In some embodiments, the vector is selected from a plasmid, a retroviral vector, a lentiviral vector, a non-replicating lentiviral vector, an adenovirus vector, or an adeno-associated virus vector.

In some embodiments, the therapeutic agent comprises, or consists essentially of, or yet further consists of a toxin, or an immune activator.

In some embodiments, the toxin comprises, or consists essentially of, or yet further consists of one or more of: a chemotherapeutic agent, a radiopharmaceutical, or a protein.

In some embodiments, the chemotherapeutic agent comprises, or consists essentially of, or yet further consists of one or more of: an alkylating agent, a nitrosourea, an antimetabolite, an anti-tumor antibiotic, a topoisomerase inhibitor, a mitotic inhibitor, or a corticosteroid.

In some embodiments, the immune activator comprises, or consists essentially of, or yet further consists of a cytokine, an immune checkpoint inhibitor, or a neoantigen target directing an immune component to the cancer cells.

In some embodiments, the cytokine comprises, or consists essentially of, or yet further consists of one or more of: IL-12Interleukin 18 (IL-18), Interleukin 15 (IL-15), Interleukin 21 (IL-21), Interleukin 27 (IL-27), C—X—C Motif Chemokine Ligand 9 (CXCL9), C—X—C Motif Chemokine Ligand 10 (CXCL10), C—X—C Motif Chemokine Ligand 11 (CXCL11), Interferon α (IFNα), Type I interferon (IFNI), Type II interferon (IFNII), Interferon γ(IFNγ), Interleukin 2 (IL-2), Interleukin 5 (IL-5), Tumor Necrosis Factor (TNF), Interferon β (IFNβ), Interleukin 18 (IL-18), CD40 Ligand (CD40L), Colony Stimulating Factor 2 (CSF2), or an equivalent of each thereof. In some embodiments, the cytokine comprises, or consists essentially of, or yet further consists of CXCL 10 and IL-12. In some embodiments, the cytokine comprises, or consists essentially of, or yet further consists of CXCL10 and IL-21. In some embodiments, the cytokine comprises, or consists essentially of, or yet further consists of CXCL10 and IL-27.

In some embodiments, the neoantigen comprises, or consists essentially of, or yet further consists of one or more of: an embryonic antigen optionally Ephrin Type-A Receptor 2 (EphA2), Interleukin 13 Receptor Subunit Alpha 2 (IL13Ra2) homodimers, carcinoembryonic antigen (CEA), or heat shock protein (hsp) gp96 (HSP96); alphafetoprotein (AFP), CA-125 (Mucin 16, Cell Surface Associated), MUC-1 (Mucin 1, Cell Surface Associated), Epithelial tumor antigen (ETA), Tyrosinase, Melanoma-associated antigen (MAGE), abnormal products of ras, p53, CD10, CD19, CD20, CD21, CD22, CD25, CD30, CD33, CD34, CD37, CD44v6, CD45, CDw52, Fms-like tyrosine kinase 3 (FLT-3, CD135), c-Kit (CD117), CSF1R (Colony Stimulating Factor 1 Receptor, CD115), CD133, PDGFR-α (CD140a), PDGFR-β (CD 140b), chondroitin sulfate proteoglycan 4 (CSPG4, melanoma-associated chondroitin sulfate proteoglycan), EGFR (Epidermal Growth Factor Receptor), de2-7-EGFR (EGFRvIII), Folate binding protein, Her2neu, Her3, PSMA (Prostate-Specific Membrane Antigen), PSCA (Prostate Stem Cell Antigen), PSA (Prostate-Specific Antigen), TAG-72, HLA-DR, IGFR, IL3R, fibroblast activating protein (FAP), Carboanhydrase IX (MN/CA IX), Carcinoembryonic antigen (CEA), EpCAM, CDCP1, Derlin1, Tenascin, frizzled 1-10, VEGFR2 (KDR/FLK1), VEGFR3 (FLT4, CD309), Endoglin, CLEC14, Tem1-8, or Tie2.

In some embodiments, the immune component comprises, or consists essentially of, or yet further consists of an immune cell specifically recognizing and binding to the neoantigen, an antibody specifically recognizing and binding to the neoantigen, or both.

In some embodiments, the immune cell comprises, or consists essentially of, or yet further consists of one or more of: an NK cell, an NKT cell, a T cell, a B cell, a dendritic cell, a splenocyte or a macrophage. In some embodiments, the immune cell expressing a chimeric antigen receptor (CAR) specifically recognizing and binding to the neoantigen.

In some embodiments, the antibody is an immune cell engager specifically recognizing and binding to the neoantigen and a cell surface marker of the immune cell.

In some embodiments, the cell surface marker of the immune cell comprises, or consists essentially of, or yet further consists of one or more of CD3, CD28, 4-1BB, CD16, NKG2D or CD64.

In some embodiments, the cancer cells are isolated from a tumor biopsy from the subject.

In some embodiments, the cancer cell is a mammalian cell. In some embodiments, the mammal is selected from a canine, a feline, an equine, or a human patient.

In some embodiments, the cancer cell is a carcinoma cell or a sarcoma cell.

In some embodiments, the cancer cell is a cell of a solid tumor. Additionally or alternatively, the cancer cell is a brain tumor cancer cell. In further embodiments, the cancer cell a glioma cancer cell.

Additionally proved is a population of isolated EVs as disclosed herein.

In other aspect, the isolated or purified EV are modified to contain or express a therapeutic agent by modifying the cancer cell to contain or express the therapeutic agent. In one aspect, the therapeutic agent is a therapeutic polynucleotide or a polynucleotide that expresses a therapeutic protein, polynucleotide or molecule. Non-limiting examples of polynucleotide include one or more or all of DNA and RNA, for example, a gene or gene fragment, exons, introns, messenger RNA (mRNA), transfer RNA, ribosomal RNA, ribozymes, cDNA, dsRNA, siRNA, miRNA, recombinant polynucleotides, branched polynucleotides, isolated DNA of any sequence, isolated RNA of any sequence, or nucleic acid probes and primers. The cancer cell can be modified to contain and express the therapeutic polynucleotide by transducing the cancer cell with a vector containing the polynucleotide. Non-limiting examples of such vectors include a plasmid, a retroviral vector, a lentiviral vector, a non-replicating lentiviral vector, an adenovirus vector, or an adeno-associated virus vector. The modification of the cells can be a transient modification. In other embodiments, the modification is a stable modification. It is contemplated that by modifying the cells prior to collection of the cell-derived vesicles released by the modified cells, one can collect exosomes containing different amounts and types of therapeutic agents. Any method for cellular modification known to one of skill in the art can be used to modify the cells.

The vector also comprises the necessary regulatory molecules for replication and/or expression of the polynucleotide, e.g., promoters and/or enhancers, as required. Biscistronic vectors can be used for expression of two or more polynucleotides or genes.

In another aspect, the therapeutic agent comprises a toxin, or an immune activator. Non-limiting examples of toxins are one or more of: a chemotherapeutic agent, a radiopharmaceutical, or a protein or an antisense polynucleotide. Non-limiting examples of immune activators include one or more of a cytokine, an immune checkpoint inhibitor, or a neoantigen target directing an immune component to the cancer cells. Non-limiting examples of chemotherapeutic agents include one or more of an alkylating agent, a nitrosourea, an antimetabolite, an anti-tumor antibiotic, a topoisomerase inhibitor, a mitotic inhibitor, or a corticosteroid. In one aspect, the cytokine comprises IL-12.

Various methods can be performed to capsulate a therapeutic agent in an EV. One non-limiting example is active (in vitro) loading, optionally by one or more of the following: sonication of EVs, such as purified EVs, with the presence of the therapeutic agent, transfecting the therapeutic agent to EVs, or electroporation of EVs, with the presence of the therapeutic agent. Another non-limiting example is passive (in vivo) loading, i.e., by creating a cell line comprising the therapeutic agent (such as, by introducing a lentivirus (LV), or an AAV, or other suitable vector, in to a cell and expressing the therapeutic agent in the cell temporarily or stably, or directly introducing the therapeutic agent into the cell), culturing the cell under conditions suitable for producing EVs, and then purifying the produced EVs loaded with the therapeutic agent.

In some embodiments, the cytokine capsulated in the EVs as a therapeutic agent comprises, or consists essentially of, or yet further consists of CXCL10, IL21, IL12, or IL27. In some embodiments, more than one (such as about two, or about three, or about four, or about five, or about ten, or more) cytokines are capsulated in the EVs as a therapeutic agent, such as any combination of CXCL10, IL21, IL12, or IL27. In some embodiments, CXCL10 is capsulated in the EVs as a therapeutic agent along with IL21. In some embodiments, CXCL10 is capsulated in the EVs as a therapeutic agent along with IL12. In some embodiments, CXCL10 is capsulated in the EVs as a therapeutic agent along with IL27. In some embodiments, the cytokine comprises, or consists essentially of, or yet further consists of an anti-cancer cytokine as disclosed herein.

In some embodiments, a detectable marker or a polynucleotide expressing the detectable marker is further capsulated in the EVs. In further embodiments, the detectable marker, such as a far red fluorescent protein, is suitable for detecting the EVs in vitro or ex vivo. In yet further embodiments, the detectable marker, such as a far red fluorescent protein, is suitable for monitoring or tracking the EVs in vivo.

In some embodiments, a detectable marker or a polynucleotide expressing the detectable marker is capsulated in the EVs as a therapeutic agent. In further embodiments, the detectable marker comprises, or consists essentially of, or yet further consists of one or more of: a surface membrane receptor or an equivalent thereof that can be specifically targeted (e.g. a non-human receptor only expressed after EV delivery to guide cytotoxic therapeutics, such as a CAR-T therapy or a CAR-NK therapy or other adoptive cellular therapy) with no off target effects, a tumor antigen (such as, Ephrin A2, IL13Ra2, muc1, EEA1, HSP96, CEA etc.), an immune checkpoint (IC) unique to a cancer (such as, neoantigen, or NB-associated protein that is expressed with Class I or Class II specificity), or a tethered cytokine (which remains in proximity with the targeted cell). In some embodiments, the surface membrane receptor or equivalent thereof comprises, or consists essentially of, or yet further consists of glycoprotein D (gD) of an HSV. Additionally or alternatively, the surface membrane receptor or equivalent thereof is non-human. In yet further embodiments, a therapy targeting the detectable marker is combined with the treatment using EVs as disclosed herein. For example, an EV comprising a polynucleotide expressing a tumor antigen can be used as described herein in delivering the polynucleotide into a cancer cell and expressing the tumor antigen on the cancer cell. Such expressed tumor antigen can be recognized by the combination therapy, such as a chimeric antigen receptor (CAR) therapy, and the cell expressing the tumor antigen can be damaged or eliminated by the combination therapy. Accordingly, the detectable marker or the polynucleotide expressing the same can be considered as a therapeutic agent as used herein.

In some embodiments, the therapeutic agent comprises, or consists essentially of, or yet further consists of an agent of a gene therapy, such as a splice-switching oligonucleotide (SSO) that modifies expression of a gene of interest, an adeno-associated virus (AAV) expressing a gene of interest, a transposon mediated transfer of a single strand (ss) DNA, etc. In some embodiments, the AAV expresses adenosine deaminase (ADA1). In some embodiments, the SSO reduces expression of CD274 (PD-L1), or Insulin Like Growth Factor 1 Receptor (IGFR), or Tumor Protein P53 (p53), or MDM2 Proto-Oncogene (MDM2).

The cancer cell or cells for the production of the modified EVs can be primary cancer cells isolated form a tumor biopsy or can be from a cell line, generated from a biopsy or obtained commercially, e.g., from the American Type Culture Collection (ATCC) or other similar cell bank. In one aspect, the cancer cell or cells to be modified are the patient's cells who will be receiving the therapy. The cells can be from any animal species, e.g., a mammalian cell such as a canine, a feline, an equine, or a human cell.

In some embodiments, the cancer cell is selected from cancers of the: circulatory system, for example, heart (sarcoma [angiosarcoma, fibrosarcoma, rhabdomyosarcoma, liposarcoma], myxoma, rhabdomyoma, fibroma, lipoma and teratoma), mediastinum and pleura, and other intrathoracic organs, vascular tumors and tumor-associated vascular tissue; respiratory tract, for example, nasal cavity and middle ear, accessory sinuses, larynx, trachea, bronchus and lung such as small cell lung cancer (SCLC), non-small cell lung cancer (NSCLC), bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; gastrointestinal system, for example, esophagus (squamous cell carcinoma, adenocarcinoma, leiomyosarcoma, lymphoma), stomach (carcinoma, lymphoma, leiomyosarcoma), gastric, pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); gastrointestinal stromal tumors and neuroendocrine tumors arising at any site; genitourinary tract, for example, kidney (adenocarcinoma, Wilm's tumor [nephroblastoma], lymphoma, leukemia), bladder and/or urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); liver, for example, hepatoma (hepatocellular carcinoma), cholangiocarcinoma, hepatoblastoma, angiosarcoma, hepatocellular adenoma, hemangioma, pancreatic endocrine tumors (such as pheochromocytoma, insulinoma, vasoactive intestinal peptide tumor, islet cell tumor and glucagonoma); bone, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; nervous system, for example, neoplasms of the central nervous system (CNS), primary CNS lymphoma, skull cancer (osteoma, hemangioma, granuloma, xanthoma, (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); reproductive system, for example, gynecological, uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma) and other sites associated with female genital organs; placenta, penis, prostate, testis, and other sites associated with male genital organs; hematologic system, for example, blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); oral cavity, for example, lip, tongue, gum, floor of mouth, palate, and other parts of mouth, parotid gland, and other parts of the salivary glands, tonsil, oropharynx, nasopharynx, pyriform sinus, hypopharynx, and other sites in the lip, oral cavity and pharynx; skin, for example, malignant melanoma, cutaneous melanoma, basal cell carcinoma, squamous cell carcinoma, Karposi's sarcoma, moles dysplastic nevi, lipoma, angioma, dermatofibroma, and keloids; adrenal glands: neuroblastoma; and other tissues comprising connective and soft tissue, retroperitoneum and peritoneum, eye, intraocular melanoma, and adnexa, breast, head or/and neck, anal region, thyroid, parathyroid, adrenal gland and other endocrine glands and related structures, secondary and unspecified malignant neoplasm of lymph nodes, secondary malignant neoplasm of respiratory and digestive systems and secondary malignant neoplasm of other sites. Additionally or alternatively, the cancer cell is a solid tumor or a liquid cancer. In some embodiments, the cancer cell is a primary cancer cell. In another embodiment, the cancer is metastatic.

Also provided are pluralities (e.g., a population) of one or more EVs, that are the same or different from each other. The population can be heterogeneous or substantially homogeneous or highly homogenous, e.g., more than 90%, or 95% or 98%, or 99% or 100% identical in composition. Methods to purify or isolate EVs are known in the art and briefly described herein.

Further provided is a composition comprising the isolated or purified EV or population and a carrier, e.g., a pharmaceutically acceptable carrier. Examples of carriers are provided herein. The compositions can contain additional agents, such as stabilizers or preservatives for storage or formulation such as lyophilization. Examples of such are known in the art and described herein.

The EVs and compositions containing same can be combined with other appropriate anti-cancer agents such as immunotherapy agents, chemotherapeutic agents, known in the art and briefly described herein.

In some embodiments, the immunotherapy agent comprises, consists essentially of, or consists of one or more selected from monoclonal antibodies (such as a monospecific, bispecific or multispecific antibody recognizing a tumor-specific antigen and/or an immune checkpoint), antibody-drug conjugates (e.g., recognizing a tumor-specific antigen and/or an immune checkpoint wherein the conjugated drug kill or damage a cancer cell expressing the tumor-specific antigen and/or inhibit an inhibitory immune checkpoint and/or active a stimulating immune checkpoint), a CAR therapy, a cell therapy (e.g., transplanting an anti-cancer immune cell optionally amplified and/or activated in vivo, or administering an immune cell expressing a chimeric antigen receptor (CAR)), immune regulators, cancer vaccines, an inhibitor or antagonist of an inhibitory immune checkpoint (referred to herein as a “checkpoint inhibitor”, such as a chemical substance, an antisense oligonucleotide (ASO), a RNA interference (RNAi), a Clustered Regularly Interspaced Short Palindromic Repeat (CRISPR) system, a vector delivering each thereof), an activator or agonist of a stimulatory immune checkpoint (such as an activating ligand). In some embodiments, the immunotherapy agent comprises, consists essentially of, or consists of one or more monoclonal antibodies, bispecific antibodies and antibody fragments. In one embodiment, the immunotherapy agent comprises, consists essentially of, or consists of one or more of bispecific antibodies specifically binding to a tumor-specific antigen and engages an immune cell, such as a bispecific T-cell engager, a bispecific NK-cell engager, a bispecific NKT-cell engager, a bispecific gamma-delta T-cell engager, and a bispecific cytotoxic T-cell engager.

In some embodiments, the immunotherapy agent comprises, consists essentially of, or consists of one or more antibody-drug conjugates. In some embodiments, the immunotherapy agent comprises, consists essentially of, or consists of one or more CAR cell therapy, such as administration of an immune cell expressing a CAR, including but not limited to CAR T cells, CAR NK cells, CAR NKT cells, CAR CD8+ T cells, CAR cytotoxic T cells, CAR gamma-delta T cells. In some embodiments, the immunotherapy agent comprises, consists essentially of, or consists of one or more cancer vaccines, such as a polypeptide or a polynucleotide mimicking a tumor-specific antigen and capable of inducing an immune response to the antigen in a subject. In some embodiments, the immunotherapy agent comprises, consists essentially of, or consists of one or more oncolytic virus therapy, such as a viral vector specifically infecting and optionally duplicating in a cancer cell and delivering an immunotherapy agent to the cancer cell. In one embodiment, the oncolytic virus is an HSV, optionally selected from HSV-1 and HSV-2. In a further embodiment, the oncolytic virus increases the expression optionally on the cell surface of a tumor-specific antigen in a cancer cell; and/or reduces the expression and/or activity of an inhibitory immune checkpoint in a cancer cell; and/or increases the expression and/or activity of a stimulatory immune checkpoint in a cancer cell.

Non-limiting examples of monoclonal antibodies include rituximab, blinatumomab, alemtuzumab, ibritumomab tiuxetan, bevacizumab, bevacizumab-awwb, cetuximab, panitumumab, ofatumumab, denosumab, pertuzumab, obinutuzumab, elotuzumab, ramucirumab, dinutuximab, daratumumab, trastuzumab, trastuzumab-dkst, nivolumab, pembrolizumab, cemiplimab, spartalizumab, camrelizumab, sintilimab, tislelizumab, toripalimab, AMF 514 (MEDI0680), balstilimab, avelumab, durvalumab, atezolizumab, ipilimumab, tremelimumab, zalifrelimab, and AGEN1181. In some embodiments, the monoclonal antibody is combined with another agent. For example, rituximab may be formulated with hyaluronidase human.

Non-limiting examples of antibody-drug conjugates include moxetumomab pasudotox-tdfk, brentuximab vedotin, trastuzumab emtansine, inotuzumab ozogamicin, gemtuzumab ozogamicin, tagraxofusp-erzs, polatuzumab vedotin-piiq, enfortumab vedotin-ejfv, trastuzumab deruxtecan, and sacituzumab govitecan-hziy.

Non-limiting examples of CAR T-cell therapy include tisagenlecleucel and axicabtagene ciloleucel.

Non-limiting examples of immune regulators include interleukins, aldesleukin, interferon alfa-2a/2b, pexidartinib, erythropoietin, granulocyte-macrophage colony-stimulating factor (GM-CSF), granulocyte colony-stimulating factor (G-CSF), thalidomide, lenalidomide, pomalidomide, and imiquimod.

Non-limiting examples of cancer vaccines include BCG live (THERACYS®) or sipuleucel-T (PROVENGE®).

Non-limiting examples of oncolytic virus therapy include oncorine (H101) and talimogene laherparepvec (IMLYGIC®).

In some embodiments, the immunotherapy agent comprises, consists essentially of, or consists of a checkpoint inhibitor.

In some embodiments, the checkpoint inhibitor comprises, consists essentially of, or consists of a non-antibody agent. In some embodiments, the checkpoint inhibitor comprises, consists essentially of, or consists of GS4224, AMP-224, CA-327, CA-170, BMS-1001, BMS-1166, peptide-57, M7824, MGD013, CX-072, UNP-12, NP-12, or a combination of two or more thereof.

In some embodiments, the checkpoint inhibitor comprises, consists essentially of, or consists of one or more selected from an anti-PD-1 agent, an anti-PD-L1 agent, an anti-CTLA-4 agent, an anti-LAG-3 agent, an anti-TIM-3 agent, an anti-TIGIT agent, an anti-VISTA agent, an anti-B7-H3 agent, an anti-BTLA agent, an anti-ICOS agent, an anti-GITR agent, an anti-4-1BB agent, an anti-OX40 agent, an anti-CD27 agent, an anti-CD28 agent, an anti-CD40 agent, and an anti-Siglec-15 agent. In some embodiments, the anti-PD-1 agent, the anti-PD-L1 agent, the anti-CTLA-4 agent, the anti-LAG-3 agent, the anti-TIM-3 agent, the anti-TIGIT agent, the anti-VISTA agent, the anti-B7-H3 agent, the anti-BTLA agent, the anti-ICOS agent, the anti-GITR agent, the anti-4-1BB agent, the anti-OX40 agent, the anti-CD27 agent, the anti-CD28 agent, the anti-CD40 agent, or the anti-Siglec-15 agent is an antagonist. In some embodiments, the anti-PD-1 agent, the anti-PD-L1 agent, the anti-CTLA-4 agent, the anti-LAG-3 agent, the anti-TIM-3 agent, the anti-TIGIT agent, the anti-VISTA agent, the anti-B7-H3 agent, the anti-BTLA agent, the anti-ICOS agent, the anti-GITR agent, the anti-4-1BB agent, the anti-OX40 agent, the anti-CD27 agent, the anti-CD28 agent, the anti-CD40 agent, or the anti-Siglec-15 agent is an agonist. In some embodiments, the anti-PD-1 agent, the anti-PD-L1 agent, the anti-CTLA-4 agent, the anti-LAG-3 agent, the anti-TIM-3 agent, the anti-TIGIT agent, the anti-VISTA agent, the anti-B7-H3 agent, the anti-BTLA agent, the anti-ICOS agent, the anti-GITR agent, the anti-4-1BB agent, the anti-OX40 agent, the anti-CD27 agent, the anti-CD28 agent, the anti-CD40 agent, or the anti-Siglec-15 agent is an inhibitor. In some embodiments, the anti-LAG-3 agent comprises, consists essentially of, or consists of AK104, KN046, eftilagimod alpha, relatlimab, LAG525, MK-4280, REGN3767, TSR-033, BI754111, Sym022, FS118, or MGD013. In some embodiments, the anti-TIM-3 agent comprises, consists essentially of, or consists of CA-327, TSR-022, MBG453, Sym023, INCAGN2390, LY3321367, BMS-986258, SHR-1702, or RO7121661. In some embodiments, the anti-TIGIT agent comprises, consists essentially of, or consists of MK-7684, etigilimab, tiragolumab, BMS-986207, AB-154, or ASP-8374. In some embodiments, the anti-VISTA agent comprises, consists essentially of, or consists of JNJ-61610588 or CA-170. In some embodiments, the anti-B7-H3 agent comprises, consists essentially of, or consists of enoblituzumab, MGD009, or omburtamab. In some embodiments, the anti-BTLA agent comprises, consists essentially of, or consists of TAB004/JS004. In some embodiments, the anti-Siglec-15 agent comprises, consists essentially of, or consists of NC318. In some embodiments, the checkpoint inhibitor comprises, consists essentially of, or consists of AK104 or KN046.

In some embodiments, the checkpoint inhibitor comprises, consists essentially of, or consists of an anti-PD1 agent or an anti-PD-L1 agent.

In some embodiments, the anti-PD1 agent comprises, consists essentially of, or consists of an anti-PD1 antibody or an antigen binding fragment thereof. In some embodiments, the anti-PD1 antibody comprises, consists essentially of, or consists of nivolumab, pembrolizumab, cemiplimab, spartalizumab, camrelizumab, sintilimab, tislelizumab, toripalimab, AMF 514 (MEDI0680), balstilimab, or a combination of two or more thereof.

In some embodiments, the anti-PD-L1 agent comprises, consists essentially of, or consists of an anti-PD-L1 antibody or an antigen binding fragment thereof. In some embodiments, the anti-PD-L1 antibody comprises, consists essentially of, or consists of avelumab, durvalumab, atezolizumab, envafolimab, or a combination of two or more thereof.

In some embodiments, the checkpoint inhibitor comprises, consists essentially of, or consists of an anti-CTLA-4 agent. In some embodiments, the anti-CTLA-4 agent comprises, consists essentially of, or consists of an anti-CTLA-4 antibody or an antigen binding fragment thereof. In some embodiments, the anti-CTLA-4 antibody comprises, consists essentially of, or consists of ipilimumab, tremelimumab, zalifrelimab, or AGEN1181, or a combination thereof.

In some embodiments, the immunotherapy agent comprises, consists essentially of, or consists of pembrolizumab, optionally in treating a non-small cell lung cancer. In a further embodiment, the pembrolizumab therapy comprises, consists essentially of, or consists of administration of pembrolizumab to a subject at a dose of 200 mg every 3 weeks. In some embodiments, the immunotherapy agent comprises, consists essentially of, or consists of nivolumab. In a further embodiment, the nivolumab therapy comprises, consists essentially of, or consists of nivolumab administration to a subject 240 mg once every 2 weeks and 480 mg once every 4 weeks. In some embodiments, the immunotherapy agent comprises, consists essentially of, or consists of ipilimumab. In a further embodiment, the ipilimumab therapy comprises, consists essentially of, or consists of administration of ipilimumab to a subject at a dose of 1, 3 or 10 mg/kg every 3 weeks for a total of 4 doses. In some embodiments, the immunotherapy agent comprises, consists essentially of, or consists of avelumab. In a further embodiment, the avelumab therapy comprises, consists essentially of, or consists of administration of avelumab at a dose of 800 mg every 2 weeks. In some embodiment, the immunotherapy agent comprises, consists essentially of, or consists of durvalumab. In a further embodiment, the durvalumab therapy comprises, consists essentially of, or consists of administration of durvalumab to a subject at a dose of 10 mg/kg every 2 weeks. In some embodiments, the immunotherapy agent comprises, consists essentially of, or consists of atezolizumab.

The compositions can also comprise a chemotherapeutic agent, such as for example, 5-fluorouracil (5-FU), pemetrexed, raltitrexed, nolatrexed, plevitrexed, GS7904L, capecitabine, methotrexate, pralatrexate, CT-900, NUC-3373, or a combination of two or more thereof.

In some embodiments, the chemotherapeutic agent comprises 5-FU based adjuvant therapy. In some embodiments, the 5-FU based adjuvant therapy comprises, consists essentially of, or consists of FOLFOX, FOLFOX-4, FOLFIRI, MOF, deflexifol, or a combination of 5-FU with one or more selected from radiation, methyl-CCNU, leucovorin, oxaliplatin (such as cisplatin), irinotecan, mitomycin, cytarabine, and levamisole. In some embodiments, the inhibitor of thymidylate biosynthesis comprises, consists essentially of, or consists of FOLFOX. In some embodiments, the inhibitor of thymidylate biosynthesis comprises, consists essentially of, or consists of FOLFOX-4. In some embodiments, the inhibitor of thymidylate biosynthesis comprises, consists essentially of, or consists of FOLFIRI. In some embodiments, the inhibitor of thymidylate biosynthesis comprises, consists essentially of, or consists of MOF. In some embodiments, the inhibitor of thymidylate biosynthesis comprises, consists essentially of, or consists of deflexifol. In some embodiments, the inhibitor of thymidylate biosynthesis comprises, consists essentially of, or consists of a combination of 5-FU with one or more selected from radiation, methyl-CCNU, leucovorin, oxaliplatin (such as cisplatin), irinotecan, mitomycin, cytarabine, and levamisole.

The EVs, populations and compositions containing same can be used in vitro to assay for acceptable therapies to treat a patient, for example to assay for personalized therapy or in vivo as a therapeutic treatment.

### Isolation of Extracellular Vesicles

The purified populations of EVs of the present disclosure can be isolated using any method known by those in the art. Non-limiting examples include differential centrifugation by ultracentrifugation (Théry et al. (2006) Curr. Protoc. Cell Biol. 30:3.22.1-3.22.29; Witmer et al. (2013) J. Extracellular v.2), sucrose gradient purification (Escola et al. (1998) J. Biol. Chem. 273:20121-20127), and combination filtration/concentration (Lamparski et al. (2002) J. Immunol. Methods 270:211-226).

The purified populations of the cell-derived vesicles disclosed herein may be purified from by a method comprising tangential flow filtration (TFF) that may contain a hollow fiber filter or a cartridge filter.

In some embodiments, the purified populations of cell-derived vesicles (e.g., exosomes and/or microvesicles) of the present disclosure can be isolated from conditioned media via direct isolation using membrane filtration devices (e.g. VivaSpin Centrifugal Concentrator, (Vivaproducts, Inc. Littleton, MA, USA)). In some embodiments, the microvesicles are isolated using tangential flow filtration and filters (e.g., a hollow fiber filtration or a cartridge filter) with size cutoffs to select for a desired microvesicle population, for example, from about 100 nm to about 1000 nm, about 200 nm to about 900 nm, about 300 nm to about 800 nm, about 400 nm to about 700 nm, about 500 nm to about 600. In some embodiments, the filters have a cutoff size of about 100 nm, about 200 nm, about 300 nm, about 400 nm, about 500 nm, about 600 nm, about 700 nm, about 800 nm, about 900 nm, or about 1000 nm. In a specific embodiment, the cell-derived vesicles of the instant disclosure have a size between about 90 nm and about 200 nm (e.g., about 90 nm, about 100 nm, about 110 nm, about 120 nm, about 130 nm, about 140 nm, about 150 nm, about 160 nm, about 170 nm, about 180 nm, about 190 nm or about 200 nm).

After isolation, the EVs can be concentrated to provide a purified population of cell-derived vesicles. Any appropriate method can be used to concentrate the cell-derived vesicles, e.g. exosomes. Non-limiting examples of such include centrifugation, ultrafiltration, filtration, differential centrifugation and column filtration with a 100 kDA to 300 kDa pore size, or either a 100 kDA to 300 kDa pore size. Further sub-populations can be isolated using antibodies or other agents that are specific for a specific marker expressed by the desired exosome population.

In some embodiments, the methods disclosed herein further comprise formulating the purified population of the EVs by mixing the population with a carrier and/or a therapeutic agent such as an anti-cancer therapy. In addition or alternatively, the exosome composition can be combined with trehalose for enhanced stability, e.g., at a concentration of about 15 nM to about 50 nM of trehalose in carrier (e.g., PBS), or alternatively about 25 nM of trehalose in carrier (e.g., PBS). Methods to formulate exosomes with trehalose are described in Bosch et al. (2016) “Trehaolose prevents aggregation of exosomes and cryodamage” Scientific Reports 6, Article number 36162, doe: 10.1038/srep36162, incorporated herein by reference.

### Formulations and Pharmaceutical Compositions

The present disclosure provides purified populations of EVs. In some embodiments, the population of cell-derived vesicles is substantially homogeneous. In other embodiments, the population of cell-derived vesicles is heterogeneous.

In some embodiments, the substantially homogeneous population is a purified population where at least 90% of the EVs have a diameter of less than 200 nm or any subrange or diameter thereof, such as about 80 nm to about 200 nm, about 80 nm to about 190 nm, about 80 nm to about 180 nm, about 80 nm to about 170 nm, about 80 nm to about 160 nm, about 80 nm to about 150 nm, about 80 nm to about 140 nm, about 80 nm to about 130 nm, about 80 nm to about 120 nm, about 80 nm to about 110 nm, about 90 nm to about 200 nm, about 90 nm to about 190 nm, about 90 nm to about 180 nm, about 90 nm to about 170 nm, about 90 nm to about 160 nm, about 90 nm to about 150 nm, about 90 nm to about 140 nm, about 90 nm to about 130 nm, about 90 nm to about 120 nm, about 90 nm to about 110 nm, or about 100 nm, optionally as determined by a NanoSight LM10HS (available from Malvern Instruments Ltd, Amesbury, MA, USA).

In some embodiments, the concentration of EVs in the population comprises between about 0.5 micrograms and 100 micrograms of EVs collected per approximately 10⁶ cells as determined by a detergent compatible (DC) assay (such as the one available from Biorad, Hercules, CA, USA). In some embodiments, the DC assay comprise a known amount of bovine serum albumin (BSA) serving as a control and calculates the EV quantity by assessing the relative protein amount of the EVs compared to the BSA. In some embodiments, the concentration of EVs in the population comprises between about 100 micrograms and 5000 micrograms of EVs collected per approximately 10⁶ cells. In other embodiments, the concentration of EVs in the population comprises between about 100 micrograms and 500 micrograms of EVs collected per approximately 10⁶ cells. In other embodiments, the concentration of EVs in the population comprises between about 500 micrograms and 1000 micrograms of EVs collected per approximately 10⁶ cells. In other embodiments, the concentration of EVs in the population comprises between about 1000 micrograms and 5000 micrograms of EVs collected per approximately 10⁶ cells. In other embodiments, the concentration of EVs in the population comprises between about 40 micrograms and 100 micrograms of EVs collected per approximately 10⁶ cells. In other embodiments, the concentration of EVs in the population comprises less than about 300 micrograms of EVs collected per approximately 10⁶ cells. In other embodiments, the concentration of EVs in the population comprises less than about 200 micrograms of EVs collected per approximately 10⁶ cells. In other embodiments, the concentration of EVs in the population comprises between about 10 micrograms and 40 micrograms of EVs collected per approximately 10⁶ cells. In yet other embodiments, the concentration of EVs in the population comprises less than about 30 micrograms of EVs collected per approximately 10⁶ cells. In yet other embodiments, the concentration of EVs in the population is less than about 20 micrograms per 10⁶ cells.

The cell-derived vesicles (e.g., microvesicles or EVs) can be purified on the basis of size. In some embodiments, EVs have a diameter of less than 200 nm or any subranges or diameters thereof, such as about 30 nm to about 200 nm, about 30 nm to about 190 nm, about 30 nm to about 180 nm, about 30 nm to about 170 nm, about 30 nm to about 160 nm, about 30 nm to about 150 nm, about 30 nm to about 140 nm, about 30 nm to about 130 nm, about 30 nm to about 120 nm, about 30 nm to about 110 nm, about 50 nm to about 200 nm, about 50 nm to about 190 nm, about 50 nm to about 180 nm, about 50 nm to about 170 nm, about 50 nm to about 160 nm, about 50 nm to about 150 nm, about 50 nm to about 140 nm, about 50 nm to about 130 nm, about 50 nm to about 120 nm, about 50 nm to about 110 nm, about 80 nm to about 200 nm, about 80 nm to about 190 nm, about 80 nm to about 180 nm, about 80 nm to about 170 nm, about 80 nm to about 160 nm, about 80 nm to about 150 nm, about 80 nm to about 140 nm, about 80 nm to about 130 nm, about 80 nm to about 120 nm, about 80 nm to about 110 nm, about 90 nm to about 200 nm, about 90 nm to about 190 nm, about 90 nm to about 180 nm, about 90 nm to about 170 nm, about 90 nm to about 160 nm, about 90 nm to about 150 nm, about 90 nm to about 140 nm, about 90 nm to about 130 nm, about 90 nm to about 120 nm, about 90 nm to about 110 nm, or about 100 nm. See, for example, Théry et al., J Extracell Vesicles. 2018 Nov. 23; 7 (1): 1535750; Doyle et al., Cells. 2019 Jul. 15; 8 (7): 727; and Brennan et al., Sci Rep. 2020 Jan. 23; 10 (1): 1039. Additionally or alternatively, the diameter of an EV is smaller than that of a microvesicle. In some embodiments, microvesicles have a diameter of up to 1000 nm or any subranges or diameters thereof, such as about 100 nm and about 1000 nm, 110 nm and about 1000 nm, 120 nm and about 1000 nm, 130 nm and about 1000 nm, 140 nm and about 1000 nm, 150 nm and about 1000 nm, 160 nm and about 1000 nm, 170 nm and about 1000 nm, 180 nm and about 1000 nm, 190 nm and about 1000 nm, or 200 nm and about 1000 nm.

In some embodiments, the concentration of EVs is determined by a NanoSite™ particle analyzer.

The purified populations of EVs can be purified based on the size of the cell-derived vesicles in the composition, such as using filtration.

The compositions disclosed herein may further comprise a carrier, for example, a pharmaceutically acceptable carrier. In some embodiments, more than one pharmaceutically acceptable carrier can be used. Any pharmaceutically acceptable carrier known to those of skill in the art can be used.

In some embodiments, the pharmaceutically acceptable carrier is a preservative, for example, a polymeric preservative or a stabilizing agent.

In some embodiments, the pharmaceutically acceptable carrier is selected from the group consisting of a polyethylene glycol (PEG) (e.g., PEG 150 Distearate), honey, a large molecular weight protein (e.g., bovine serum albumin or soy protein), polyvinyl alcohol, glyceryl monostearate, hyaluronic acid, glycerin, preferably vegetable-derived, proteins, preferably hydrolyzed proteins, (e.g., soy protein and silk protein), vasoline, citrosept, parabens, xanthan gum, i-carregaan, phytagel, Carbopol® polymers, and polyvinyl pyrrolidone.

In some embodiments, exosomes are preserved in serum albumin. Non-limiting examples of serum albumins appropriate for preservation of exosomes include bovine serum albumin (BSA), human serum albumin (HSA), ovalbumin (OVA), and lactalbumin.

Biocompatible gelation agents include thermosensitive sol-gel reversible hydrogels such as aqueous solutions of poloxamers. In one aspect, the poloxamer is a nonionic triblock copolymer composed of a central hydrophobic chain of polyoxypropylene (e.g., (poly(propylene oxide)) flanked by two hydrophilic chains of polyoxyethylene (e.g., poly(ethylene oxide)). In one aspect, poloxamer has the formula

HO(C₂H₄O)_{b}(C₃H₆O)ₐ(C₂H₄O)_{b}OH

wherein a is from 10 to 100, 20 to 80, 25 to 70, or 25 to 70, or from 50 to 70; b is from 5 to 250, 10 to 225, 20 to 200, 50 to 200, 100 to 200, or 150 to 200. In another aspect, the poloxamer has a molecular weight from 2,000 to 15,000, 3,000 to 14,000, or 4,000 to 12,000. Poloxamers useful herein are sold under the tradename Pluronic® manufactured by BASF. Non-limiting examples of poloxamers useful herein include, but are not limited to, Pluronic® F68, P103, P105, P123, F127, and L121.

In one aspect, the biocompatible gelation agent is an agent that is liquid prior to application to a subject (e.g., at room temperature or colder) and becomes a gel after application to the subject (e.g., at body temperature). In one embodiment, the biocompatible gelation agent is a hydrogel.

In another aspect, disclosed herein is a composition comprising EVs and a poloxamer wherein the composition is in a sol (liquid) phase at about 0° C. to about 20° C. and transitions a gel (solid) phase at or near the body temperature or higher, such as about 25° C. to about 40° C., or about 30° C. to about 37° C.

In some aspects, the pharmaceutically acceptable carrier is a pharmaceutically acceptable aqueous carrier such as water or an aqueous carrier. Examples of pharmaceutically acceptable aqueous carrier include sterile water, saline, phosphate buffered saline, aqueous hyaluronic acid, Ringer's solution, dextrose solution, Hank's solution, and other aqueous physiologically balanced salt solutions. In some embodiments, the pharmaceutically acceptable aqueous carrier is Normosol™—R.

Nonaqueous pharmaceutically acceptable carriers include, fixed oils, vegetable oils such as olive oil and sesame oil, triglycerides, propylene glycol, polyethylene glycol, and injectable organic esters such as ethyl oleate can also be used.

Pharmaceutically acceptable carrier can also contain minor amounts of additives, such as substances that enhance isotonicity, chemical stability, or cellular stability. Examples of buffers include phosphate buffer, bicarbonate buffer and Tris buffer, while examples of preservatives include thimerosol, cresols, formalin and benzyl alcohol. In certain aspects, the pH can be modified depending upon the mode of administration. In some aspect, the composition has a pH in the physiological pH range, such as pH 7 to 9.

In one aspect, depending on the type of a pharmaceutically acceptable carrier used, the compositions described herein can comprise about 0.1-100%, 0.1-50%, or 0.1-30%, such as 0.1%, 0.25%, 0.5%, 0.75%, 1%, 2%, 5%, 7%, 10%, 15%, 20%, 25%, 30%, 40%, 45%, 50%, 55%, 60%, 65%, 70%, 75%, 80%, 85%, 90% or 95% of the pharmaceutically acceptable carrier used in the total weight of the composition, or any range between two of the numbers (end point inclusive).

In some embodiments, any one of the above listed pharmaceutically acceptable carriers is expressly excluded.

In some embodiments, the EVs are frozen (e.g., snap-frozen) or freeze-dried (e.g., lyophilized) to promote stability, preserve activity and increase shelf-life. One skilled in the art would understand how to reconstitute the lyophilized product before use.

In some embodiments, the populations of EVs described herein are used immediately after isolation. In other embodiments, the populations of EVs are cryopreserved (e.g. frozen), for example, using any cryopreservation techniques well-known to those skilled in the art. In some embodiments, all or substantially of the cells and/or cellular debris are removed from the culture medium prior to cryopreservation. In some embodiments, all or substantially of the cells and/or cellular debris are removed from the culture medium after cryopreservation.

### Applications and Uses

Disclosed herein are methods for making EVs that can be used in the methods of the instant disclosure. In some embodiments, the EVs are obtained from cancer cells. In some embodiments, the cancer cells are engineered to express a therapeutic agent as described herein, wherein the therapeutic agent is loaded into the EVs. In some embodiments, the therapeutic agent is loaded into the EVs using a mechanical method, e.g., sonication.

The EVs and compositions containing same are useful in a method for delivering an agent to a tumor/cancer cell by contacting the cell with the agent-loaded EV.

In some embodiments, the agent is a therapeutic agent as described herein. In some embodiments, the agent is a polynucleotide. In some embodiments, the polynucleotide is an expression cassette or an expression vector.

In some embodiments, the agent is a virus. In some embodiments, the virus within the EVs is shielded from a subject's immune response when administered to the subject. In some embodiments, the virus is from the parvoviridae family. In some embodiments, the virus from the parvoviridae family is selected from the following genera: Amdoparvovirus, Aveparvovirus, Bocaparvovirus, Copiparvovirus, Dependoparvovirus, Erythroparvovirus, Protoparvovirus, and Tetraparvovirus. In a specific embodiment the virus is from the genus Dependoparvovirus. In some embodiments, the virus from the genus Dependoparvovirus is an adeno-associated virus (AAV). In some embodiments, the AAV is selected from the group consisting of AAV1, AAV2, AAV3, AAV4, AAV5, AAV6, AAV7, AAV8, AAV9, AAV10, AAV11, AAV12, AAV rh. 10 and AAVrh74.

The EVs and compositions containing same are useful in a method for inhibiting the growth or metastasis of a cancer cell comprising, or consisting essentially of, or yet further consisting of contacting the cancer cell with an effective amount of the EVs and compositions as described herein, thereby inhibiting the growth of, or metastasis of the cancer or cancer cell. In one aspect, the contacting is in vitro, and can be used to test if the EV therapy, alone or in combination with other therapies, can inhibit the growth of or metastasis of the cancer or cancer cell. It also can be used in pre-clinical studies to determine effective amounts of mono- or combination therapies. The contacting can be in vivo, and provide a therapy as described herein.

The amount to be delivered is an effective amount which can be determined by the treating veterinarian or treating physician, and will vary with the cancer to be treated, the staging of the cancer, the health and age of the subject or patient. In one aspect, the EVs are isolated from cancer cells originally isolated from the subject to be treated, which are then modified to contain the EVs comprising the therapeutic agent.

Also provided herein is a method of treating a cancer in a subject in need thereof comprising, or consisting essentially of, or yet further consisting of administering to the subject an effective amount of extracellular vesicles (EVs) comprising a therapeutic agent of this disclosure thereby treating the subject. In one aspect, the EVs are isolated from a tumor or cancer cells of the subject and modified to comprise the therapeutic agent. In some embodiments, the EVs are produced by cancer cells that are of the same type as the cancer in the subject. In some embodiments, the EVs isolated from one type of cancer is used to treat the same type of cancer in a subject. In some embodiments, the EVs are produced and isolated from cancer cells obtained from the subject (e.g., through a biopsy). In some embodiments, the EVs are produced by cancer cells that are of a different type as the cancer in the subject. In some embodiments, the EVs isolated from one type of cancer is used to treat a different type of cancer. Modes of administration are described herein, and can be systemic or local to the tumor in the subject. The cancer for treatment can be primary, metastatic or relapsed.

In one aspect, provided herein is a method for treating a cancer or tumor in a subject by isolating cancer cells from the subject and modifying the cells to express a therapeutic agent in the EVs of the cells and isolating the EVs and then administering an effective amount of the EVs to the subject.

In one aspect, provided is a method for either or both of the following: (a) treating a cancer in a subject in need thereof, or (b) conferring or inducing an immune response to a cancer in a subject in need thereof. The method comprises, or consists essentially of, or yet further consists of administering to the subject, for example an effective amount of, extracellular vesicles (EVs) isolated from cancer cells of the subject.

In some embodiments, the EVs comprise a therapeutic agent. In some embodiments, the EVs further comprise a surface membrane associated protein that selectively targets a cancer cell. In some embodiments, the isolated cancer cells are engineered to produce the therapeutic agent in the EVs.

In some embodiments, the therapeutic agent comprises, or consists essentially of, or yet further consists of a therapeutic polynucleotide. In some embodiments, the therapeutic polynucleotide comprises or expresses a splice-switching oligonucleotide (SSO) or an antisense polynucleotide. In further embodiments, the SSO or antisense polynucleotide reduces the expression of an oncogene or an immune suppressor in a cell of the cancer to be treated. In yet further embodiments, the oncogene comprises, or consists essentially of, or yet further consists of one or more of: Insulin Like Growth Factor 1 Receptor (IGFR), Tumor Protein P53 (p53), or MDM2 Proto-Oncogene (MDM2). In some embodiments, the immune suppressor comprises, or consists essentially of, or yet further consists of PD-L1. In some embodiments, the therapeutic polynucleotide expresses a protein toxin or an immune activator.

In some embodiments, the EVs comprise a vector comprising, or consisting essentially of, or yet further consisting of the polynucleotide. In some embodiments, the vector is selected from a plasmid, a retroviral vector, a lentiviral vector, a non-replicating lentiviral vector, an adenovirus vector, or an adeno-associated virus vector.

In some embodiments, the therapeutic agent comprises, or consists essentially of, or yet further consists of a toxin, or an immune activator.

In some embodiments, the toxin comprises, or consists essentially of, or yet further consists of one or more of: a chemotherapeutic agent, a radiopharmaceutical, or a protein toxin.

In some embodiments, the chemotherapeutic agent comprises, or consists essentially of, or yet further consists of one or more of: an alkylating agent, a nitrosourea, an antimetabolite, an anti-tumor antibiotic, a topoisomerase inhibitor, a mitotic inhibitor, or a corticosteroid.

In some embodiments, the immune activator comprises, or consists essentially of, or yet further consists of a cytokine, an immune checkpoint inhibitor, or a neoantigen target directing an immune component to the cancer cells.

In some embodiments, the cytokine comprises, or consists essentially of, or yet further consists of one or more of: Interleukin 12 (IL-12), Interleukin 18 (IL-18), Interleukin 15 (IL-15), Interleukin 21 (IL-21), Interleukin 27 (IL-27), C—X—C Motif Chemokine Ligand 9 (CXCL9), C—X—C Motif Chemokine Ligand 10 (CXCL10), C—X—C Motif Chemokine Ligand 11 (CXCL11), Interferon α (IFNα), Type I interferon (IFNI), Type II interferon (IFNII), Interferon γ (IFNγ), Interleukin 2 (IL-2), Interleukin 5 (IL-5), Tumor Necrosis Factor (TNF), Interferon β (IFNβ), Interleukin 18 (IL-18), CD40 Ligand (CD40L), Colony Stimulating Factor 2 (CSF2), or an equivalent of each thereof. In some embodiments, the cytokine comprises, or consists essentially of, or yet further consists of CXCL10 and IL-12. In some embodiments, the cytokine comprises, or consists essentially of, or yet further consists of CXCL10 and IL-21. In some embodiments, the cytokine comprises, or consists essentially of, or yet further consists of CXCL10 and IL-27.

In some embodiments, the neoantigen comprises, or consists essentially of, or yet further consists of one or more of: an embryonic antigen optionally Ephrin Type-A Receptor 2 (EphA2), Interleukin 13 Receptor Subunit Alpha 2 (IL13Ra2) homodimers, carcinoembryonic antigen (CEA), or heat shock protein (hsp) gp96 (HSP96); alphafetoprotein (AFP), CA-125 (Mucin 16, Cell Surface Associated), MUC-1 (Mucin 1, Cell Surface Associated), Epithelial tumor antigen (ETA), Tyrosinase, Melanoma-associated antigen (MAGE), abnormal products of ras, p53, CD10, CD19, CD20, CD21, CD22, CD25, CD30, CD33, CD34, CD37, CD44v6, CD45, CDw52, Fms-like tyrosine kinase 3 (FLT-3, CD135), c-Kit (CD117), CSF1R (Colony Stimulating Factor 1 Receptor, CD115), CD133, PDGFR-α (CD140a), PDGFR-β (CD 140b), chondroitin sulfate proteoglycan 4 (CSPG4, melanoma-associated chondroitin sulfate proteoglycan), EGFR (Epidermal Growth Factor Receptor), de2-7-EGFR (EGFRvIII), Folate binding protein, Her2neu, Her3, PSMA (Prostate-Specific Membrane Antigen), PSCA (Prostate Stem Cell Antigen), PSA (Prostate-Specific Antigen), TAG-72, HLA-DR, IGFR, IL3R, fibroblast activating protein (FAP), Carboanhydrase IX (MN/CA IX), Carcinoembryonic antigen (CEA), EpCAM, CDCP1, Derlin1, Tenascin, frizzled 1-10, VEGFR2 (KDR/FLK1), VEGFR3 (FLT4, CD309), Endoglin, CLEC14, Tem1-8, or Tie2.

In some embodiments, the immune component comprises, or consists essentially of, or yet further consists of an immune cell specifically recognizing and binding to the neoantigen, an antibody specifically recognizing and binding to the neoantigen, or both.

In some embodiments, the immune cell comprises, or consists essentially of, or yet further consists of one or more of: an NK cell, an NKT cell, a T cell, a B cell, a dendritic cell, a splenocyte or a macrophage. In some embodiments, the immune cell expressing a chimeric antigen receptor (CAR) specifically recognizing and binding to the neoantigen.

In some embodiments, the antibody is an immune cell engager specifically recognizing and binding to the neoantigen and a cell surface marker of the immune cell. In some embodiments, the cell surface marker of the immune cell comprises, or consists essentially of, or yet further consists of one or more of CD3, CD28, 4-1BB, CD16, NKG2D or CD64.

In some embodiments, the EVs can cross the blood-brain barrier and target tumors or metastases in the central nervous system.

In some embodiments, the isolated cancer cells are from a tumor biopsy of the subject. In some embodiments, the isolated cancer cells are cancer cells of the central nervous system. In some embodiments, the cancer cells of the central nervous system comprise glioblastomas or gliomas.

In some embodiments, the method further comprises resection of the cancer in the subject prior to the administration.

In some embodiments, the administration is systemically or locally to a tumor in the subject. In a specific embodiment, the administration is systemic administration.

In some embodiments, the cancer is primary, metastatic or relapsed. In some embodiments, the cancer to be treated comprises, or consists essentially of, or yet further consists of a solid tumor. Additional or alternatively, the cancer to be treated comprises, or consists essentially of, or yet further consists of a brain tumor. In further embodiments, the cancer to be treated comprises, or consists essentially of, or yet further consists of a glioma.

In some embodiments, the method is selected from a first line therapy, a second line therapy, a third line therapy, a fourth line therapy or a fifth line therapy.

In some embodiments, the administration is repeated.

In some embodiments, the method further comprises administering an anti-cancer therapy to the subject. In some embodiments, the anti-cancer therapy comprises, or consists essentially of, or yet further comprises of one or more of: a surgical resection of the cancer, a radiation therapy, a chemotherapy, or an immunotherapy.

In some embodiments, the subject is a mammal. In further embodiments, the mammal is selected from a canine, a feline, an equine, or a human patient.

In some embodiments, the cancer is selected from a carcinoma or a sarcoma.

Additionally provided is a composition comprising, or consisting essentially of, or yet further consisting of an isolated EV as disclosed herein or a population thereof as disclosed herein, and a carrier. In some embodiments, the carrier is a pharmaceutically acceptable carrier. In some embodiments, the EVs are the same or different from each other. In some embodiments, the composition further comprises an anti-cancer agent.

Additionally provided is a kit comprising, or consisting essentially of, or yet further consisting of one or more of: an isolated EV as disclosed herein, a population as disclosed herein, or a composition as disclosed herein, and instructions for use.

The methods of this disclosure can be combined with other effective therapies, e.g., surgical resection of the cancer or tumor, a radiation therapy, a chemotherapy, or an immunotherapy. These are known in the art and briefly described herein.

The therapies can be a first line therapy, a second line therapy, a third line therapy, a fourth line therapy or a fifth line therapy. Administration can be a single dose or repeated as necessary.

The EVs and compositions containing same can be combined with other appropriate anti-cancer agents such as immunotherapy agents, chemotherapeutic agents, known in the art and briefly described herein. In some embodiments, the EVs are loaded with an immunotherapy agent. In some embodiments, the EVs are loaded with a chemotherapeutic agent.

In some embodiments, the immunotherapy agent comprises, consists essentially of, or consists of one or more selected from monoclonal antibodies (such as a monospecific, bispecific or multispecific antibody recognizing a tumor-specific antigen and/or an immune checkpoint), antibody-drug conjugates (e.g., recognizing a tumor-specific antigen and/or an immune checkpoint wherein the conjugated drug kill or damage a cancer cell expressing the tumor-specific antigen and/or inhibit an inhibitory immune checkpoint and/or active a stimulating immune checkpoint), a CAR therapy, a cell therapy (e.g., transplanting an anti-cancer immune cell optionally amplified and/or activated in vivo, or administering an immune cell expressing a chimeric antigen receptor (CAR)), immune regulators, cancer vaccines, an inhibitor or antagonist of an inhibitory immune checkpoint (referred to herein as a “checkpoint inhibitor”, such as a chemical substance, an antisense oligonucleotide (ASO), a RNA interference (RNAi), a Clustered Regularly Interspaced Short Palindromic Repeat (CRISPR) system, a vector delivering each thereof), an activator or agonist of a stimulatory immune checkpoint (such as an activating ligand). In some embodiments, the immunotherapy agent comprises, consists essentially of, or consists of one or more monoclonal antibodies, bispecific antibodies and antibody fragments. In one embodiment, the immunotherapy agent comprises, consists essentially of, or consists of one or more of bispecific antibodies specifically binding to a tumor-specific antigen and engages an immune cell, such as a bispecific T-cell engager, a bispecific NK-cell engager, a bispecific NKT-cell engager, a bispecific gamma-delta T-cell engager, and a bispecific cytotoxic T-cell engager. In some embodiments, the immunotherapy agent comprises, consists essentially of, or consists of one or more antibody-drug conjugates. In some embodiments, the immunotherapy agent comprises, consists essentially of, or consists of one or more CAR cell therapy, such as administration of an immune cell expressing a CAR, including but not limited to CAR T cells, CAR NK cells, CAR NKT cells, CAR CD8+ T cells, CAR cytotoxic T cells, CAR gamma-delta T cells. In some embodiments, the immunotherapy agent comprises, consists essentially of, or consists of one or more cancer vaccines, such as a polypeptide or a polynucleotide mimicking a tumor-specific antigen and capable of inducing an immune response to the antigen in a subject. In some embodiments, the immunotherapy agent comprises, consists essentially of, or consists of one or more oncolytic virus therapy, such as a viral vector specifically infecting and optionally duplicating in a cancer cell and delivering an immunotherapy agent to the cancer cell. In one embodiment, the oncolytic virus is an HSV, optionally selected from HSV-1 and HSV-2. In a further embodiment, the oncolytic virus increases the expression optionally on the cell surface of a tumor-specific antigen in a cancer cell; and/or reduces the expression and/or activity of an inhibitory immune checkpoint in a cancer cell; and/or increases the expression and/or activity of a stimulatory immune checkpoint in a cancer cell.

Non-limiting examples of monoclonal antibodies include rituximab, blinatumomab, alemtuzumab, ibritumomab tiuxetan, bevacizumab, bevacizumab-awwb, cetuximab, panitumumab, ofatumumab, denosumab, pertuzumab, obinutuzumab, elotuzumab, ramucirumab, dinutuximab, daratumumab, trastuzumab, trastuzumab-dkst, nivolumab, pembrolizumab, cemiplimab, spartalizumab, camrelizumab, sintilimab, tislelizumab, toripalimab, AMF 514 (MEDI0680), balstilimab, avelumab, durvalumab, atezolizumab, ipilimumab, tremelimumab, zalifrelimab, and AGEN1181. In some embodiments, the monoclonal antibody is combined with another agent. For example, rituximab may be formulated with hyaluronidase human.

Non-limiting examples of antibody-drug conjugates include moxetumomab pasudotox-tdfk, brentuximab vedotin, trastuzumab emtansine, inotuzumab ozogamicin, gemtuzumab ozogamicin, tagraxofusp-erzs, polatuzumab vedotin-piiq, enfortumab vedotin-ejfv, trastuzumab deruxtecan, and sacituzumab govitecan-hziy.

Non-limiting examples of CAR T-cell therapy include tisagenlecleucel and axicabtagene ciloleucel.

Non-limiting examples of immune regulators include interleukins, aldesleukin, interferon alfa-2a/2b, pexidartinib, erythropoietin, granulocyte-macrophage colony-stimulating factor (GM-CSF), granulocyte colony-stimulating factor (G-CSF), thalidomide, lenalidomide, pomalidomide, and imiquimod.

Non-limiting examples of cancer vaccines include BCG live (THERACYS®) or sipuleucel-T (PROVENGE®).

Non-limiting examples of oncolytic virus therapy include oncorine (H101) and talimogene laherparepvec (IMLYGIC®).

In some embodiments, the immunotherapy agent comprises, consists essentially of, or consists of a checkpoint inhibitor.

In some embodiments, the checkpoint inhibitor comprises, consists essentially of, or consists of a non-antibody agent. In some embodiments, the checkpoint inhibitor comprises, consists essentially of, or consists of GS4224, AMP-224, CA-327, CA-170, BMS-1001, BMS-1166, peptide-57, M7824, MGD013, CX-072, UNP-12, NP-12, or a combination of two or more thereof.

In some embodiments, the checkpoint inhibitor comprises, consists essentially of, or consists of one or more selected from an anti-PD-1 agent, an anti-PD-L1 agent, an anti-CTLA-4 agent, an anti-LAG-3 agent, an anti-TIM-3 agent, an anti-TIGIT agent, an anti-VISTA agent, an anti-B7-H3 agent, an anti-BTLA agent, an anti-ICOS agent, an anti-GITR agent, an anti-4-1BB agent, an anti-OX40 agent, an anti-CD27 agent, an anti-CD28 agent, an anti-CD40 agent, and an anti-Siglec-15 agent. In some embodiments, the anti-PD-1 agent, the anti-PD-L1 agent, the anti-CTLA-4 agent, the anti-LAG-3 agent, the anti-TIM-3 agent, the anti-TIGIT agent, the anti-VISTA agent, the anti-B7-H3 agent, the anti-BTLA agent, the anti-ICOS agent, the anti-GITR agent, the anti-4-1BB agent, the anti-OX40 agent, the anti-CD27 agent, the anti-CD28 agent, the anti-CD40 agent, or the anti-Siglec-15 agent is an antagonist. In some embodiments, the anti-PD-1 agent, the anti-PD-L1 agent, the anti-CTLA-4 agent, the anti-LAG-3 agent, the anti-TIM-3 agent, the anti-TIGIT agent, the anti-VISTA agent, the anti-B7-H3 agent, the anti-BTLA agent, the anti-ICOS agent, the anti-GITR agent, the anti-4-1BB agent, the anti-OX40 agent, the anti-CD27 agent, the anti-CD28 agent, the anti-CD40 agent, or the anti-Siglec-15 agent is an agonist. In some embodiments, the anti-PD-1 agent, the anti-PD-L1 agent, the anti-CTLA-4 agent, the anti-LAG-3 agent, the anti-TIM-3 agent, the anti-TIGIT agent, the anti-VISTA agent, the anti-B7-H3 agent, the anti-BTLA agent, the anti-ICOS agent, the anti-GITR agent, the anti-4-1BB agent, the anti-OX40 agent, the anti-CD27 agent, the anti-CD28 agent, the anti-CD40 agent, or the anti-Siglec-15 agent is an inhibitor. In some embodiments, the anti-LAG-3 agent comprises, consists essentially of, or consists of AK104, KN046, eftilagimod alpha, relatlimab, LAG525, MK-4280, REGN3767, TSR-033, BI754111, Sym022, FS118, or MGD013. In some embodiments, the anti-TIM-3 agent comprises, consists essentially of, or consists of CA-327, TSR-022, MBG453, Sym023, INCAGN2390, LY3321367, BMS-986258, SHR-1702, or RO7121661. In some embodiments, the anti-TIGIT agent comprises, consists essentially of, or consists of MK-7684, etigilimab, tiragolumab, BMS-986207, AB-154, or ASP-8374. In some embodiments, the anti-VISTA agent comprises, consists essentially of, or consists of JNJ-61610588 or CA-170. In some embodiments, the anti-B7-H3 agent comprises, consists essentially of, or consists of enoblituzumab, MGD009, or omburtamab. In some embodiments, the anti-BTLA agent comprises, consists essentially of, or consists of TAB004/JS004. In some embodiments, the anti-Siglec-15 agent comprises, consists essentially of, or consists of NC318. In some embodiments, the checkpoint inhibitor comprises, consists essentially of, or consists of AK104 or KN046.

In some embodiments, the checkpoint inhibitor comprises, consists essentially of, or consists of an anti-PD1 agent or an anti-PD-L1 agent.

In some embodiments, the anti-PD1 agent comprises, consists essentially of, or consists of an anti-PD1 antibody or an antigen binding fragment thereof. In some embodiments, the anti-PD1 antibody comprises, consists essentially of, or consists of nivolumab, pembrolizumab, cemiplimab, spartalizumab, camrelizumab, sintilimab, tislelizumab, toripalimab, AMF 514 (MEDI0680), balstilimab, or a combination of two or more thereof.

In some embodiments, the anti-PD-L1 agent comprises, consists essentially of, or consists of an anti-PD-L1 antibody or an antigen binding fragment thereof. In some embodiments, the anti-PD-L1 antibody comprises, consists essentially of, or consists of avelumab, durvalumab, atezolizumab, envafolimab, or a combination of two or more thereof. In some embodiments, the anti-PD-L1 agent comprises, consists essentially of, or consists of an anti-PD-L1 antisense oligonucleotide.

In some embodiments, the checkpoint inhibitor comprises, consists essentially of, or consists of an anti-CTLA-4 agent. In some embodiments, the anti-CTLA-4 agent comprises, consists essentially of, or consists of an anti-CTLA-4 antibody or an antigen binding fragment thereof. In some embodiments, the anti-CTLA-4 antibody comprises, consists essentially of, or consists of ipilimumab, tremelimumab, zalifrelimab, or AGEN1181, or a combination thereof.

In some embodiments, the immunotherapy agent comprises, consists essentially of, or consists of pembrolizumab, optionally in treating a non-small cell lung cancer. In a further embodiment, the pembrolizumab therapy comprises, consists essentially of, or consists of administration of pembrolizumab to a subject at a dose of 200 mg every 3 weeks. In some embodiments, the immunotherapy agent comprises, consists essentially of, or consists of nivolumab. In a further embodiment, the nivolumab therapy comprises, consists essentially of, or consists of nivolumab administration to a subject 240 mg once every 2 weeks and 480 mg once every 4 weeks. In some embodiments, the immunotherapy agent comprises, consists essentially of, or consists of ipilimumab. In a further embodiment, the ipilimumab therapy comprises, consists essentially of, or consists of administration of ipilimumab to a subject at a dose of 1, 3 or 10 mg/kg every 3 weeks for a total of 4 doses. In some embodiments, the immunotherapy agent comprises, consists essentially of, or consists of avelumab. In a further embodiment, the avelumab therapy comprises, consists essentially of, or consists of administration of avelumab at a dose of 800 mg every 2 weeks. In some embodiment, the immunotherapy agent comprises, consists essentially of, or consists of durvalumab. In a further embodiment, the durvalumab therapy comprises, consists essentially of, or consists of administration of durvalumab to a subject at a dose of 10 mg/kg every 2 weeks. In some embodiments, the immunotherapy agent comprises, consists essentially of, or consists of atezolizumab.

The compositions can also comprise a chemotherapeutic agent, such as for example, 5-fluorouracil (5-FU), pemetrexed, raltitrexed, nolatrexed, plevitrexed, GS7904L, capecitabine, methotrexate, pralatrexate, CT-900, NUC-3373, or a combination of two or more thereof.

In some embodiments, the chemotherapeutic agent comprises 5-FU based adjuvant therapy. In some embodiments, the 5-FU based adjuvant therapy comprises, consists essentially of, or consists of FOLFOX, FOLFOX-4, FOLFIRI, MOF, deflexifol, or a combination of 5-FU with one or more selected from radiation, methyl-CCNU, leucovorin, oxaliplatin (such as cisplatin), irinotecan, mitomycin, cytarabine, and levamisole. In some embodiments, the inhibitor of thymidylate biosynthesis comprises, consists essentially of, or consists of FOLFOX. In some embodiments, the inhibitor of thymidylate biosynthesis comprises, consists essentially of, or consists of FOLFOX-4. In some embodiments, the inhibitor of thymidylate biosynthesis comprises, consists essentially of, or consists of FOLFIRI. In some embodiments, the inhibitor of thymidylate biosynthesis comprises, consists essentially of, or consists of MOF. In some embodiments, the inhibitor of thymidylate biosynthesis comprises, consists essentially of, or consists of deflexifol. In some embodiments, the inhibitor of thymidylate biosynthesis comprises, consists essentially of, or consists of a combination of 5-FU with one or more selected from radiation, methyl-CCNU, leucovorin, oxaliplatin (such as cisplatin), irinotecan, mitomycin, cytarabine, and levamisole.

The subject may be a mammal, for example, a human or non-human mammals such as a bovine, an ovine, or a porcine. In preferred embodiments, the subject is a human patient. In a further aspect, the subject has been selected for the therapy by diagnostic criteria as determined by the treating physician or health care professional.

In some embodiments, the cancer cell is selected from cancers of the: circulatory system, for example, heart (sarcoma [angiosarcoma, fibrosarcoma, rhabdomyosarcoma, liposarcoma], myxoma, rhabdomyoma, fibroma, lipoma and teratoma), mediastinum and pleura, and other intrathoracic organs, vascular tumors and tumor-associated vascular tissue; respiratory tract, for example, nasal cavity and middle ear, accessory sinuses, larynx, trachea, bronchus and lung such as small cell lung cancer (SCLC), non-small cell lung cancer (NSCLC), bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; gastrointestinal system, for example, esophagus (squamous cell carcinoma, adenocarcinoma, leiomyosarcoma, lymphoma), stomach (carcinoma, lymphoma, leiomyosarcoma), gastric, pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); gastrointestinal stromal tumors and neuroendocrine tumors arising at any site; genitourinary tract, for example, kidney (adenocarcinoma, Wilm's tumor [nephroblastoma], lymphoma, leukemia), bladder and/or urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); liver, for example, hepatoma (hepatocellular carcinoma), cholangiocarcinoma, hepatoblastoma, angiosarcoma, hepatocellular adenoma, hemangioma, pancreatic endocrine tumors (such as pheochromocytoma, insulinoma, vasoactive intestinal peptide tumor, islet cell tumor and glucagonoma); bone, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; nervous system, for example, neoplasms of the central nervous system (CNS), primary CNS lymphoma, skull cancer (osteoma, hemangioma, granuloma, xanthoma, (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); reproductive system, for example, gynecological, uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma) and other sites associated with female genital organs; placenta, penis, prostate, testis, and other sites associated with male genital organs; hematologic system, for example, blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; oral cavity, for example, lip, tongue, gum, floor of mouth, palate, and other parts of mouth, parotid gland, and other parts of the salivary glands, tonsil, oropharynx, nasopharynx, pyriform sinus, hypopharynx, and other sites in the lip, oral cavity and pharynx; skin, for example, malignant melanoma, cutaneous melanoma, basal cell carcinoma, squamous cell carcinoma, Karposi's sarcoma, moles dysplastic nevi, lipoma, angioma, dermatofibroma, and keloids; adrenal glands: neuroblastoma; and other tissues comprising connective and soft tissue, retroperitoneum and peritoneum, eye, intraocular melanoma, and adnexa, breast, head or/and neck, anal region, thyroid, parathyroid, adrenal gland and other endocrine glands and related structures, secondary and unspecified malignant neoplasm of lymph nodes, secondary malignant neoplasm of respiratory and digestive systems and secondary malignant neoplasm of other sites. Additionally or alternatively, the cancer is a solid tumor or a liquid cancer. In some embodiments, the cancer is a primary cancer. In another embodiment, the cancer is metastatic.

### Dosages and Dosing Regimens

The appropriate amount and dosing regimen of the EVs to be administered to the subject according to any of the methods disclosed herein, may be determined by one of ordinary skill in the art.

In some embodiments, the EVs alone or in combination with other therapies are administered to a subject suffering from abnormal cell growth, such as a human, either alone or as part of a pharmaceutically acceptable formulation, once a week, once a day, twice a day, three times a day, or four times a day, or even more frequently.

Administration of the EVs disclosed herein may be effected by any method that enables delivery of the compounds to the site of action. These methods include oral routes, intraduodenal routes, parenteral injection (including intravenous, subcutaneous, intramuscular, intravascular or infusion), topical, and rectal administration. Bolus doses can be used, or infusions over a period of 1, 2, 3, 4, 5, 10, 15, 20, 30, 60, 90, 120 or more minutes, or any intermediate time period can also be used, as can infusions lasting 3, 4, 5, 6, 7, 8, 9, 10, 12, 14 16, 20, 24 or more hours or lasting for 1-7 days or more. Infusions can be administered by drip, continuous infusion, infusion pump, metering pump, depot formulation, or any other suitable means.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the subjects to be treated; each unit containing a predetermined quantity of active compound calculated to produce the desired therapeutic effect in association with the required pharmaceutical carrier. The specification for the dosage unit forms of the disclosure are dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present disclosure.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present disclosure encompasses intra-patient dose-escalation as determined by the skilled artisan. Determining appropriate dosages and regimens for administration of the chemotherapeutic agent are well-known in the relevant art and would be understood to be encompassed by the skilled artisan once provided the teachings disclosed herein.

### Kits

The agents described herein may, in some embodiments, be assembled into pharmaceutical or diagnostic or research kits to facilitate their use in therapeutic, diagnostic or research applications. A kit may include one or more containers housing the components of the invention and instructions for use. Specifically, such kits may include one or more agents described herein, along with instructions describing the intended application and the proper use of these agents. In certain embodiments agents in a kit may be in a pharmaceutical formulation and dosage suitable for a particular application and for a method of administration of the agents. Kits for research purposes may contain the components in appropriate concentrations or quantities for running various experiments.

The kit may be designed to facilitate use of the methods described herein and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. In some embodiments, the compositions may be provided in a preservation solution (e.g., cryopreservation solution). Non-limiting examples of preservation solutions include DMSO, paraformaldehyde, and CryoStor® (Stem Cell Technologies, Vancouver, Canada). In some embodiments, the preservation solution contains an amount of metalloprotease inhibitors.

As used herein, “instructions” can define a component of instruction and/or promotion, and typically involve written instructions on or associated with packaging of the invention. Instructions also can include any oral or electronic instructions provided in any manner such that a user will clearly recognize that the instructions are to be associated with the kit, for example, audiovisual (e.g., videotape, DVD, etc.), internet, and/or web-based communications, etc. The written instructions may be in a form prescribed by a governmental agency regulating the manufacture, use or sale of pharmaceuticals or biological products, which instructions can also reflect approval by the agency of manufacture, use or sale for animal administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be in the form of a liquid, gel or solid (powder). The agents may be prepared sterilely, packaged in syringe and shipped refrigerated. Alternatively it may be housed in a vial or other container for storage. A second container may have other agents prepared sterilely. Alternatively the kit may include the active agents premixed and shipped in a syringe, vial, tube, or other container. The kit may have one or more or all of the components required to administer the agents to a subject, such as a syringe, topical application devices, or IV needle tubing and bag.

### Exemplary Embodiments

The instant disclosure provides the following embodiments.

Embodiment 1. A method for either or both of the following: (a) treating a cancer in a subject in need thereof, or (b) conferring or inducing an immune response to a cancer in a subject in need thereof, the method comprising administering to the subject an effective amount of extracellular vesicles (EVs) isolated from cancer cells of the subject, and wherein the EVs comprise a therapeutic agent.

Embodiment 2. The method of embodiment 1, wherein the EVs further comprise a surface membrane associated protein that selectively targets a cancer cell.

Embodiment 3. The method of embodiment 1 or embodiment 2, wherein the isolated cancer cells are engineered to produce the therapeutic agent in the EVs.

Embodiment 4. The method of any one of embodiments 1-3, wherein the therapeutic agent is a therapeutic polynucleotide.

Embodiment 5. The method of embodiment 4, wherein the therapeutic polynucleotide comprises or expresses a splice-switching oligonucleotide (SSO) or an antisense polynucleotide, and wherein the SSO or antisense polynucleotide reduces the expression of an oncogene or an immune suppressor in a cell of the cancer to be treated.

Embodiment 6. The method of embodiment 5, wherein the oncogene comprises one or more of: Insulin Like Growth Factor 1 Receptor (IGFR), Tumor Protein P53 (p53), or MDM2 Proto-Oncogene (MDM2).

Embodiment 7. The method of embodiment 5, wherein the immune suppressor comprises PD-L1.

Embodiment 8. The method of embodiment 4, wherein the therapeutic polynucleotide expresses a protein toxin or an immune activator.

Embodiment 9. The method of any one of embodiments 4 to 8, wherein the EVs comprise a vector comprising the polynucleotide.

Embodiment 10. The method of embodiment 9, wherein the vector is selected from a plasmid, a retroviral vector, a lentiviral vector, a non-replicating lentiviral vector, an adenovirus vector, or an adeno-associated virus vector.

Embodiment 11. The method of any one of embodiments 1 to 10, wherein the therapeutic agent comprises a toxin, or an immune activator.

Embodiment 12. The method of embodiment 11, wherein the toxin comprises one or more of: a chemotherapeutic agent, a radiopharmaceutical, or a protein toxin.

Embodiment 13. The method of embodiment 12, wherein the chemotherapeutic agent comprises one or more of: an alkylating agent, a nitrosourea, an antimetabolite, an anti-tumor antibiotic, a topoisomerase inhibitor, a mitotic inhibitor, or a corticosteroid.

Embodiment 14. The method of any one of embodiments 8 to 11, wherein the immune activator comprises a cytokine, an immune checkpoint inhibitor, or a neoantigen target directing an immune component to the cancer cells.

Embodiment 15. The method of embodiment 14, wherein the cytokine comprises one or more of: Interleukin 12 (IL-12), Interleukin 18 (IL-18), Interleukin 15 (IL-15), Interleukin 21 (IL-21), Interleukin 27 (IL-27), C—X—C Motif Chemokine Ligand 9 (CXCL9), C—X—C Motif Chemokine Ligand 10 (CXCL10), C—X—C Motif Chemokine Ligand 11 (CXCL11), Interferon α (IFNα), Type I interferon (IFNI), Type II interferon (IFNII), Interferon γ (IFNγ), Interleukin 2 (IL-2), Interleukin 5 (IL-5), Tumor Necrosis Factor (TNF), Interferon β (IFNβ), Interleukin 18 (IL-18), CD40 Ligand (CD40L), Colony Stimulating Factor 2 (CSF2), or an equivalent of each thereof.

Embodiment 16. The method of embodiment 14 or 15, wherein the cytokine comprises CXCL10 and IL-12.

Embodiment 17. The method of embodiment 14 or 15, wherein the cytokine comprises CXCL10 and IL-21.

Embodiment 18. The method of embodiment 14 or 15, wherein the cytokine comprises CXCL10 and IL-27.

Embodiment 19. The method of embodiment 14, wherein the neoantigen comprises one or more of: an embryonic antigen optionally Ephrin Type-A Receptor 2 (EphA2), Interleukin 13 Receptor Subunit Alpha 2 (IL13Ra2) homodimers, carcinoembryonic antigen (CEA), or heat shock protein (hsp) gp96 (HSP96);
alphafetoprotein (AFP), CA-125 (Mucin 16, Cell Surface Associated), MUC-1 (Mucin 1, Cell Surface Associated), Epithelial tumor antigen (ETA), Tyrosinase, Melanoma-associated antigen (MAGE), abnormal products of ras, p53, CD10, CD19, CD20, CD21, CD22, CD25, CD30, CD33, CD34, CD37, CD44v6, CD45, CDw52, Fms-like tyrosine kinase 3 (FLT-3, CD135), c-Kit (CD117), CSF1R (Colony Stimulating Factor 1 Receptor, CD115), CD133, PDGFR-α (CD140a), PDGFR-β (CD 140b), chondroitin sulfate proteoglycan 4 (CSPG4, melanoma-associated chondroitin sulfate proteoglycan), EGFR (Epidermal Growth Factor Receptor), de2-7-EGFR (EGFRvIII), Folate binding protein, Her2neu, Her3, PSMA (Prostate-Specific Membrane Antigen), PSCA (Prostate Stem Cell Antigen), PSA (Prostate-Specific Antigen), TAG-72, HLA-DR, IGFR, IL3R, fibroblast activating protein (FAP), Carboanhydrase IX (MN/CA IX), Carcinoembryonic antigen (CEA), EpCAM, CDCP1, Derlin1, Tenascin, frizzled 1-10, VEGFR2 (KDR/FLK1), VEGFR3 (FLT4, CD309), Endoglin, CLEC14, Tem1-8, or Tie2.

Embodiment 20. The method of embodiment 14 or embodiment 19, wherein the immune component comprises an immune cell specifically recognizing and binding to the neoantigen, an antibody specifically recognizing and binding to the neoantigen, or both.

Embodiment 21. The method of embodiment 20, wherein the immune cell comprises one or more of: an NK cell, an NKT cell, a T cell, a B cell, a dendritic cell, a splenocyte or a macrophage.

Embodiment 22. The method of embodiment 20 or 21, wherein the immune cell expressing a chimeric antigen receptor (CAR) specifically recognizing and binding to the neoantigen.

Embodiment 23. The method of embodiment 20, wherein the antibody is an immune cell engager specifically recognizing and binding to the neoantigen and a cell surface marker of the immune cell.

Embodiment 24. The method of embodiment 23, wherein the cell surface marker of the immune cell comprises one or more of CD3, CD28, 4-1BB, CD16, NKG2D or CD64.

Embodiment 25. The method of any one of embodiments 1 to 24, wherein the isolated cancer cells are from a tumor biopsy of the subject.

Embodiment 26. The method of any one of embodiments 1 to 25, further comprising resection of the cancer in the subject prior to the administration.

Embodiment 27. The method of any one of embodiments 1 to 26, wherein the administration is systemically or locally to a tumor in the subject.

Embodiment 28. The method of any one of embodiments 1 to 27, wherein the cancer is primary, metastatic or relapsed.

Embodiment 29. The method of any one of embodiments 1 to 28, wherein the cancer to be treated comprises a solid tumor, optionally a brain tumor, and further optionally a glioma.

Embodiment 30. The method of any one of embodiments 1 to 29, wherein the method is selected from a first line therapy, a second line therapy, a third line therapy, a fourth line therapy or a fifth line therapy.

Embodiment 31. The method of any one of embodiments 1 to 30, wherein the administration is repeated.

Embodiment 32. The method of any one of embodiments 1 to 31, further comprising administering an anti-cancer therapy to the subject.

Embodiment 33. The method of embodiment 32, wherein the anti-cancer therapy comprises one or more of: a surgical resection of the cancer, a radiation therapy, a chemotherapy, or an immunotherapy.

Embodiment 34. The method of any one of embodiments 1 to 33, wherein the subject is a mammal.

Embodiment 35. The method of embodiment 34, wherein the mammal is selected from a canine, a feline, an equine, or a human patient.

Embodiment 36. The method of any one of embodiments 1 to 35, wherein the cancer is selected from a carcinoma or a sarcoma.

Embodiment 37. An isolated extracellular vesicle (EV) isolated from a cancer cell, wherein the EV comprises a therapeutic agent.

Embodiment 38. The isolated EV of embodiment 37, wherein the EVs further comprise a surface membrane associated protein that selectively targets a cancer cell.

Embodiment 39. The isolated EV of embodiment 37 or 38, wherein the cancer cells are engineered to produce the therapeutic agent in the EVs.

Embodiment 40. The isolated EV of any one of embodiments 37 to 39, wherein the therapeutic agent is a therapeutic polynucleotide.

Embodiment 41. The isolated EV of embodiment 40, wherein the therapeutic polynucleotide comprises or expresses a splice-switching oligonucleotide (SSO) or an antisense polynucleotide, and wherein the SSO or antisense polynucleotide reduces the expression of an oncogene or an immune suppressor in a cell of the cancer to be treated.

Embodiment 42. The isolated EV of embodiment 41, wherein the oncogene comprises one or more of: Insulin Like Growth Factor 1 Receptor (IGFR), Tumor Protein P53 (p53), or MDM2 Proto-Oncogene (MDM2).

Embodiment 43. The isolated EV of embodiment 41, wherein the immune suppressor comprises PD-L1.

Embodiment 44. The isolated EV of embodiment 40, wherein the therapeutic polynucleotide expresses a protein toxin or an immune activator.

Embodiment 45. The isolated EV of any one of embodiments 40 to 44, comprising a vector that comprises the polynucleotide.

Embodiment 46. The isolated EV of embodiment 45, wherein the vector is selected from a plasmid, a retroviral vector, a lentiviral vector, a non-replicating lentiviral vector, an adenovirus vector, or an adeno-associated virus vector.

Embodiment 47. The isolated EV of any of embodiments 37 to 46, wherein the therapeutic agent comprises a toxin, or an immune activator.

Embodiment 48. The isolated EV of embodiment 47, wherein the toxin comprises one or more of: a chemotherapeutic agent, a radiopharmaceutical, or a protein.

Embodiment 49. The isolated EV of embodiment 48, wherein the chemotherapeutic agent comprises one or more of: an alkylating agent, a nitrosourea, an antimetabolite, an anti-tumor antibiotic, a topoisomerase inhibitor, a mitotic inhibitor, or a corticosteroid.

Embodiment 50. The isolated EV of embodiment 47, wherein the immune activator comprises a cytokine, an immune checkpoint inhibitor, or a neoantigen target directing an immune component to the cancer cells.

Embodiment 51. The isolated EV of embodiment 50, wherein the cytokine comprises one or more of: Interleukin 12 (IL-12), Interleukin 18 (IL-18), Interleukin 15 (IL-15), Interleukin 21 (IL-21), Interleukin 27 (IL-27), C—X—C Motif Chemokine Ligand 9 (CXCL9), C—X—C Motif Chemokine Ligand 10 (CXCL10), C—X—C Motif Chemokine Ligand 11 (CXCL11), Interferon α (IFNα), Type I interferon (IFNI), Type II interferon (IFNII), Interferon γ (IFNγ), Interleukin 2 (IL-2), Interleukin 5 (IL-5), Tumor Necrosis Factor (TNF), Interferon β (IFNβ), Interleukin 18 (IL-18), CD40 Ligand (CD40L), Colony Stimulating Factor 2 (CSF2), or an equivalent of each thereof.

Embodiment 52. The isolated EV of embodiment 50 or embodiment 51, wherein the cytokine comprises CXCL10 and IL-12.

Embodiment 53. The isolated EV of embodiment 50 or embodiment 51, wherein the cytokine comprises CXCL 10 and IL-21.

Embodiment 54. The isolated EV of embodiment 50 or embodiment 51, wherein the cytokine comprises CXCL 10 and IL-27.

Embodiment 55. The isolated EV of embodiment 50, wherein the neoantigen comprises one or more of: an embryonic antigen optionally Ephrin Type-A Receptor 2 (EphA2), Interleukin 13 Receptor Subunit Alpha 2 (IL13Ra2) homodimers, carcinoembryonic antigen (CEA), or heat shock protein (hsp) gp96 (HSP96); alphafetoprotein (AFP), CA-125 (Mucin 16, Cell Surface Associated), MUC-1 (Mucin 1, Cell Surface Associated), Epithelial tumor antigen (ETA), Tyrosinase, Melanoma-associated antigen (MAGE), abnormal products of ras, p53, CD10, CD19, CD20, CD21, CD22, CD25, CD30, CD33, CD34, CD37, CD44v6, CD45, CDw52, Fms-like tyrosine kinase 3 (FLT-3, CD135), c-Kit (CD117), CSF1R (Colony Stimulating Factor 1 Receptor, CD115), CD133, PDGFR-α (CD140a), PDGFR-β (CD 140b), chondroitin sulfate proteoglycan 4 (CSPG4, melanoma-associated chondroitin sulfate proteoglycan), EGFR (Epidermal Growth Factor Receptor), de2-7-EGFR (EGFRVIII), Folate binding protein, Her2neu, Her3, PSMA (Prostate-Specific Membrane Antigen), PSCA (Prostate Stem Cell Antigen), PSA (Prostate-Specific Antigen), TAG-72, HLA-DR, IGFR, IL3R, fibroblast activating protein (FAP), Carboanhydrase IX (MN/CA IX), Carcinoembryonic antigen (CEA), EpCAM, CDCP1, Derlin1, Tenascin, frizzled 1-10, VEGFR2 (KDR/FLK1), VEGFR3 (FLT4, CD309), Endoglin, CLEC14, Tem1-8, or Tie2.

Embodiment 56. The isolated EV of embodiment 50, wherein the immune component comprises an immune cell specifically recognizing and binding to the neoantigen, an antibody specifically recognizing and binding to the neoantigen, or both.

Embodiment 57. The isolated EV of embodiment 56, wherein the immune cell comprises one or more of: an NK cell, an NKT cell, a T cell, a B cell, a dendritic cell, a splenocyte or a macrophage.

Embodiment 58. The isolated EV of embodiment 56 or embodiment 57, wherein the immune cell expressing a chimeric antigen receptor (CAR) specifically recognizing and binding to the neoantigen.

Embodiment 59. The isolated EV of embodiment 56, wherein the antibody is an immune cell engager specifically recognizing and binding to the neoantigen and a cell surface marker of the immune cell.

Embodiment 60. The isolated EV of embodiment 59, wherein the cell surface marker of the immune cell comprises one or more of CD3, CD28, 4-1BB, CD16, NKG2D or CD64.

Embodiment 61. The isolated EV of any of embodiments 37 to 60, wherein the cancer cells are isolated from a tumor biopsy from the subject.

Embodiment 62. The isolated EV of any of embodiments 37 to 61, wherein the cancer cell is a mammalian cell.

Embodiment 63. The isolated EV of embodiment 62, wherein the mammal is selected from a canine, a feline, an equine, or a human patient.

Embodiment 64. The isolated EV of any of embodiments 37 to 63, wherein the cancer cell is a carcinoma cell or a sarcoma cell.

Embodiment 65. The isolated EV of any of embodiments 37 to 63, wherein the cancer cell is a cell of a solid tumor, optionally a brain tumor, and further optionally a glioma.

Embodiment 66. A population of isolated EVs of any of claims 37 to 65.

Embodiment 67. A composition comprising the isolated EV of any of embodiments 37 to 65 or the population of embodiment 66, and a carrier.

Embodiment 68. The composition of embodiment 67, wherein the carrier is a pharmaceutically acceptable carrier.

Embodiment 69. The composition of embodiment 67 or 68, wherein the EVs are the same or different from each other.

Embodiment 70. The composition of any one of embodiments 67 to 69, further comprising an anti-cancer agent.

Embodiment 71. A kit comprising one or more of: the isolated EV of any one of embodiments 37 to 65, the population to embodiment 66, or the composition of any one of embodiments 67 to 70, and instructions for use.

The following example is provided to illustrate, but not limit the scope of this disclosure.

### Experimental Example No. 1

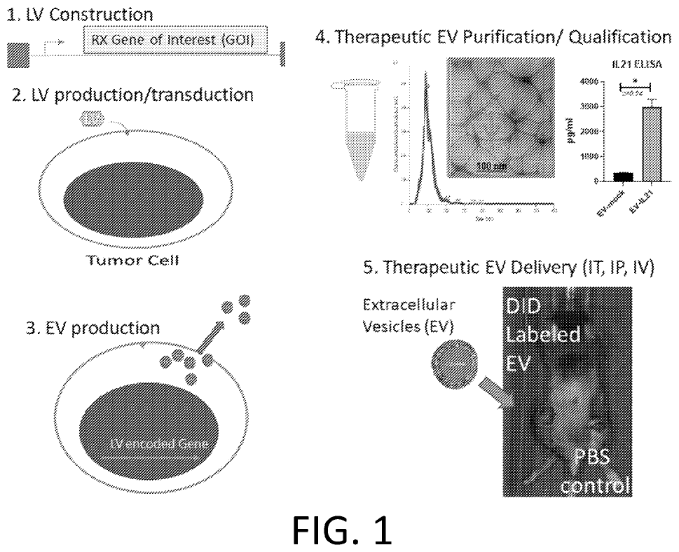

Applicant has shown that the extracellular vesicles (EVs) can be loaded with a therapeutic agent (See FIG. 1). A major advantage of the approach is that EVs are from the cell and therefore unlike a virus would not be seen as foreign and would be relatively immune inert (unless loaded with immune active molecules).

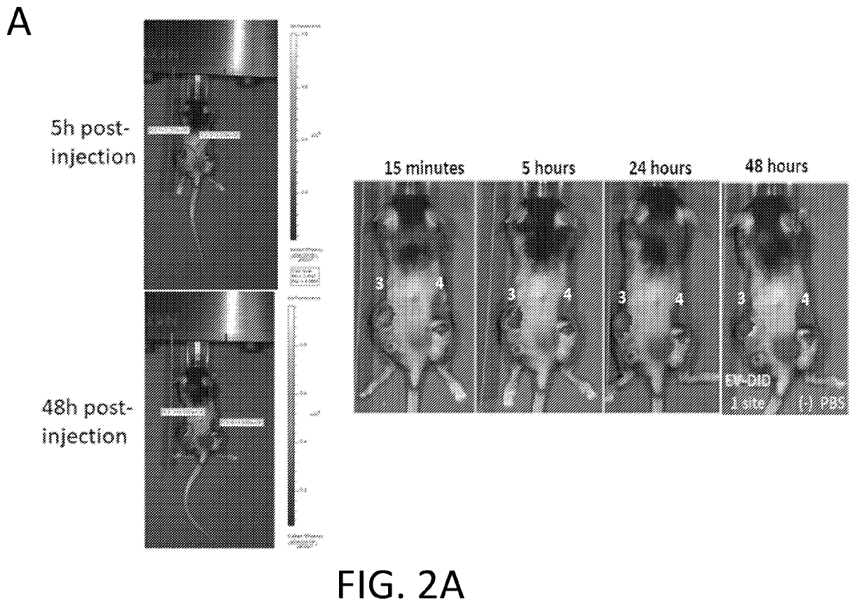

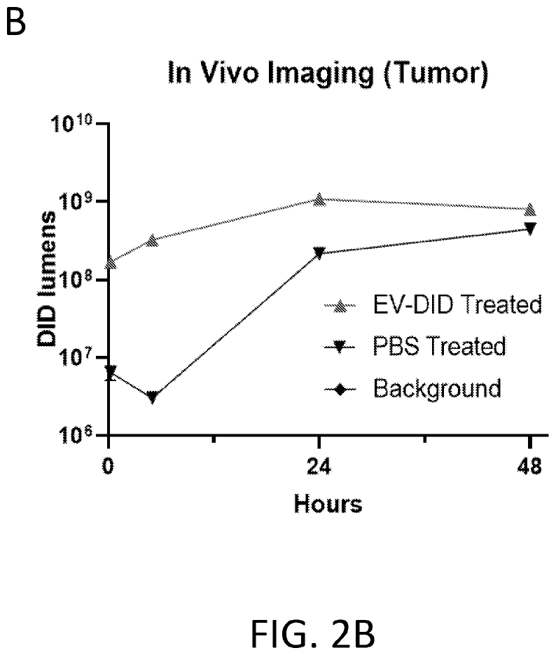

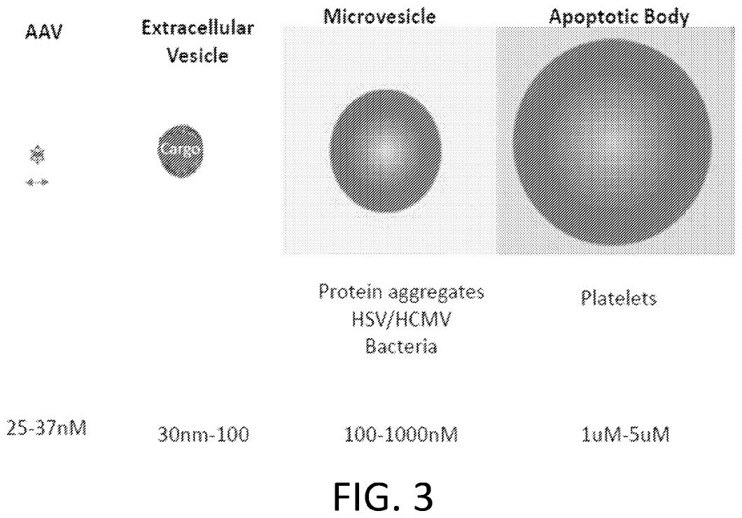

This disclosure provides methods to:
Purify EVs;
Load them with a therapeutic polynucleotide, using a vector such as a non-replicating lentiviruses (LVs) construct; and
Deliver the EV to the tumor allowing either systemic delivery and they are picked up by the tumor the EVs can be directly injected into a tumor where it redistributes to other tumors (see FIG. 2);

FIG. 2 is an image that shows IVIS imaging 15 minutes to 5h after EV injection or 24-48h later. These data show that DID labeled EVs re-distribute to the contralateral tumor that was not injected and suggests that the EVs prefer other tumor cells (specificity).

In one aspect, a method is provided wherein a patient has a cancer biopsy or tumor resection, and the tissue is transduced with an engineered LV (that encodes any of the following: a genetic toxin, cytokine, immune activator, neoantigen target, anti-sense oligo like Dawn Chandler is developing, gene therapy agent). Also provided are tumor lines from that biopsy that produces the EV encased therapeutic that allows customized therapeutic to treat that patient's cancer and may be selectively taken up by their cancer.

### Experimental Example No. 2-Isolation of Exosomes and Other Methods

For isolation of extracellular vesicles, CT2A cells at >80% confluence were washed three times with PBS and incubated in exosome free FBS medium for 24 hours. The medium was collected extracellular vesicles were isolated using “The ME™ Kit for Exosome Isolation (Urine/Media)” (New England Peptide Inc., Gardner, MA, USA), following the user's manual. Briefly, the medium was filtered through a 0.2 μm syringe filter and 20 μl of Vn96 stock was added to the tube and incubated overnight at 4° C. with end to end rotation. The tubes were centrifuged at 15,000G for 30 min at room temperature (RT). After removing the supernatant, the pellet was re-dissolved in 1 ml of PBS and centrifuged again at 15,000G for 30 min. The resultant supernatant was discarded and the pellet was diluted in PBS (1-2 ml) and passed through a 300 kDa cut-off filter. Nanoparticle Tracking Analysis was used for determination of the size, distribution and relative concentration of EVs or analyzed by Western Blot. These were the control unloaded EVs.

For cytokines, passive loading of EVs was done using CT2A cells infected with lentivirus encoding human CXCL10-IL21. The resultant cells expressing human CXCL10-IL21 were passaged 3-4 times before collecting the supernatant for extracting EVs as described above.

The EV protein concentration was determined for each batch using the BCA method (Thermo Fisher) and the concentration of human CXCL10 or human IL21 was determined using ELISA assay kits from Biolegend, following the manufacturer's instruction manuals. 100-200 μg EVs (protein) was used for the in vitro fluorescence assay. 100-200 ng of EVs (equivalent of hIL21) was coated with DiD surface dye as per user manual and used for the in vivo IVIS assays.

For AAV-GFP studies, the control CT2A EVs were incubated with the AAV-GFP virus and sonicated to facilitate the entry of virus into the EVs. The suspension was treated with proteinase to get rid of the residual virus from the surface of EVs and washed 3 times with PBS before downstream experiments.

### Experimental Example No. 3-A Non-Viral Cytokine Delivery Approach to Improve Tumor Associated Immunotherapeutic Response

Without wishing to be bound by the theory, it is hypothesized that chemokine and cytokine loaded EVs provides an effective cancer therapy platform and enables precise immunotherapy delivery to target tumor cells.

As illustrated in FIG. 1, a lentivirus (LV) expressing human CXCL10-hIL21 cassette was used to infect CT2A cells. The EVs extracted from these CT2A cells were analyzed by Nanosight and ELISA assays for human IL-21 (hIL21). The EVs were then surface coated with DiD dye for in vivo tracking studies in mice using IVIS.

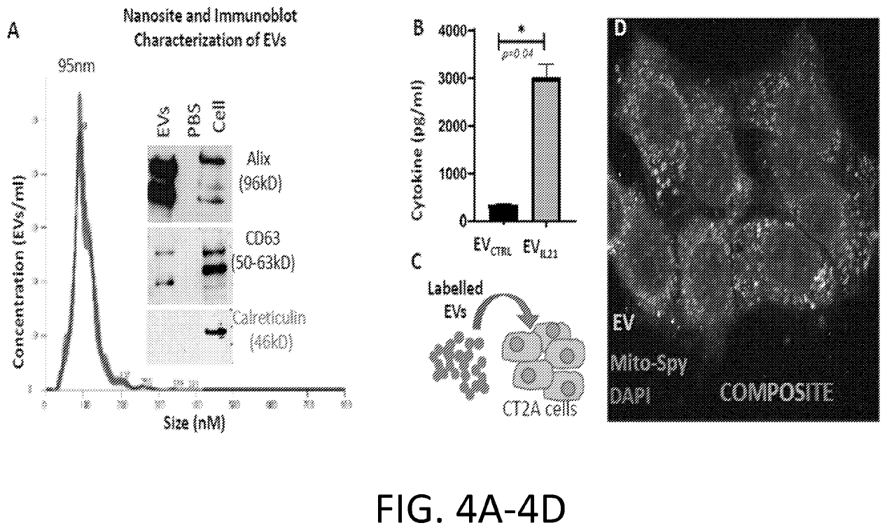

Average diameter of the isolated EVs (100 nm) was determined using Nanosite and Nanoparticle Tracking Analysis (NTA). See, for example, FIG. 4A. Further validation was performed by western blot of the isolated EVs (50 μg protein equivalent) confirming expression of EV specific positive markers-ALIX (96 kDa) and CD63 (63 kDa) and absence of negative marker-calreticulin (40 kD) (FIG. 4A, inset). The EVs extracted from these CT2A cells were analyzed by ELISA assays for human IL-21 activity and it was observed that the loaded EVs showed 3000 pg/ml level of activity per 200 ng protein equivalent of EVs. See, for example, FIG. 4B. To determine the uptake of the EVs in CT2A cells, the extracted EVs were surface labelled with DiO and incubated with CT2A cells cultured on a coverslip. See, for example, FIG. 4C. The cells were labelled with MitoSpy CMX ROS after 48 hours and washed with PBS before mounting in DAPI mounting medium. Confocal microscopy of the cover slips revealed that DiO labelled green EVs were localized towards the ER and in the cytoplasm in the EV treated cells and this was absent from untreated normal CT2A cells. See, for example, FIG. 4D. This confirms that the EVs were successfully isolated and loaded, and their integration into cells can be tracked in vitro.

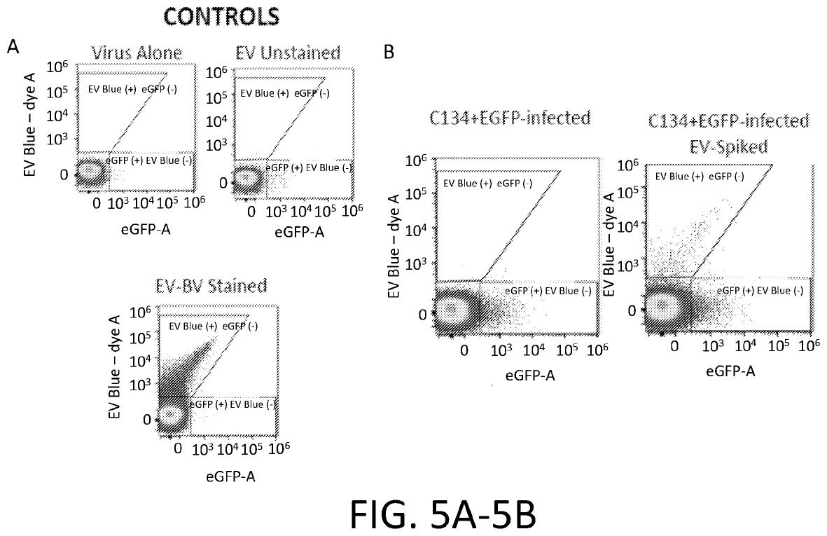

EVs were stained with ExoGlo Blue Dye to label the EV protein and the EVs were quantified using Attune Flow Cytometer. The EVs were detectable on the flow cytometer. See, for example, FIG. 5.

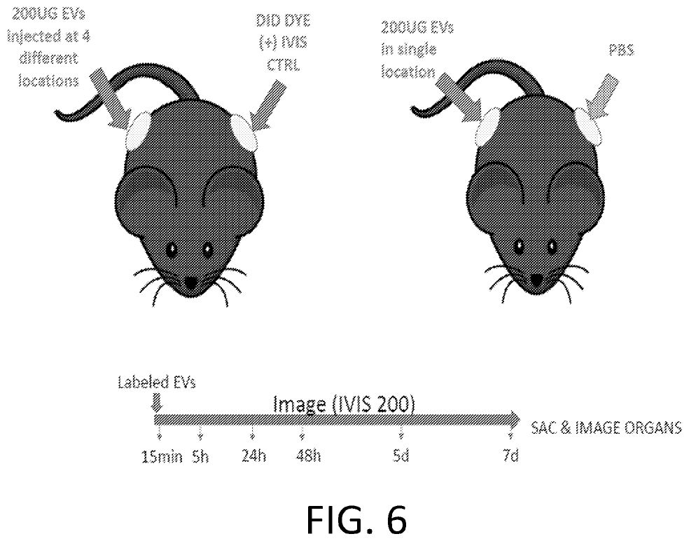

FIG. 6 provides a schematic showing CT2A mouse treatment plan. Mouse 1 (on the left side of FIG. 6) was treated with 4 fractionated doses (totaling 200 μg of hCXCL10-IL21 DID-labeled EVs: equivalent to 100 ng of hIL21) intratumorally to the right tumor) and was injected in the left tumor with DID dye as a positive control for IVIS imaging. The other mouse (on the right side of FIG. 6) was treated with 200 μg of EV (hCXCL10-IL21 DID-labeled EVs: 100 ng of hIL2) as a single dose and the contralateral side injected with PBS (negative control). The mice were imaged at 15 minutes, 5 hours, 24 hours, 48 hours, 5 days and 7 days post injection to monitor and detect EVs in vivo. The results obtained are further described below.

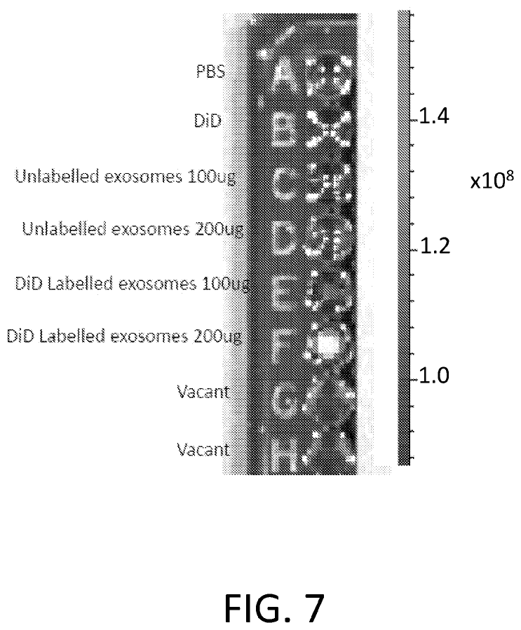

100 and 200 μg EV samples were diluted in 100 μl of phosphate-buffered saline (PBS) and imaged using IVIS 200 small animal imaging system (PerkinElmer, Waltham, MA, USA). The following setting was applied and used for imaging: Ex filter—640 nm/Em filter—700 nm with background subtraction. Average intensity within the entire circle area of each well was calculated. The fluorescence signal correlated linearly with the EV concentration. A representative result is shown in FIG. 7. Absence of fluorescence signal in unlabeled EVs shows that the EVs did not show any autofluorescence. DiD dye alone also does not fluoresce proving that unless the dye is incorporated in an EV or a cell, there is no background signal that it could generate complicating the analyses of signals in vivo.

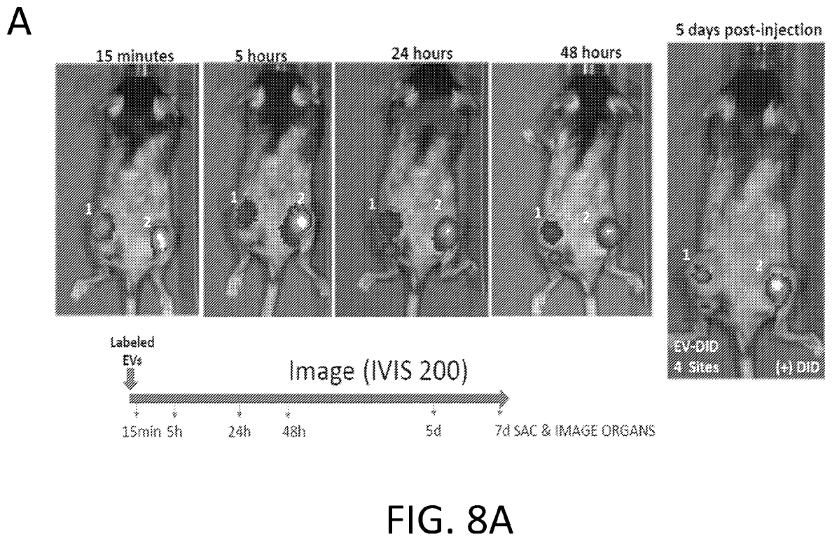

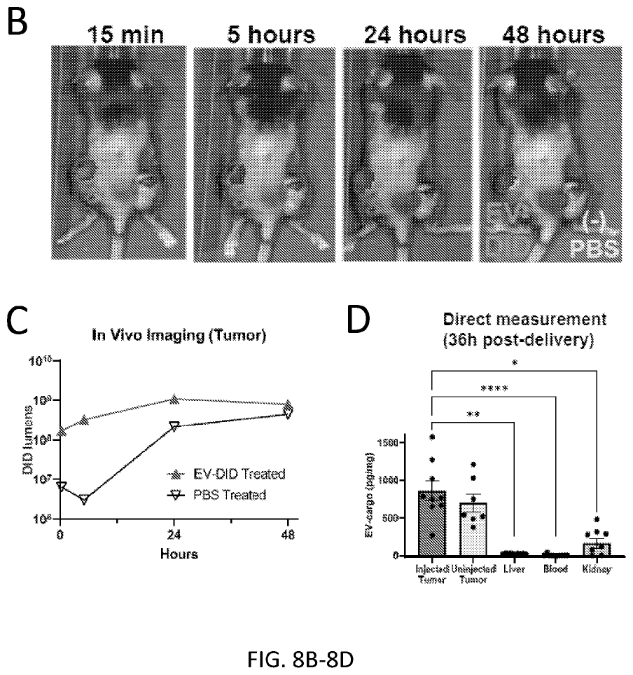

In an effort to visualize the labelled EVs in vivo by optical imaging (OI) on the whole body of live mice, a mouse flank tumor CT2A model was used on an IVIS system. EVs expressing 100 ng equivalent of human IL21 were injected intratumorally about 2 weeks (100 mm³ tumor volume) following the induction of tumor. A representative result is shown in FIG. 8A. Fluorescent signal in the tumors was seen in all the tumors. The signal started to diminish at the day 5 time point and had completely disappeared from all the tumors at the day 7 time point. No fluorescence was observed on any other parts of the mice at any of the time points.

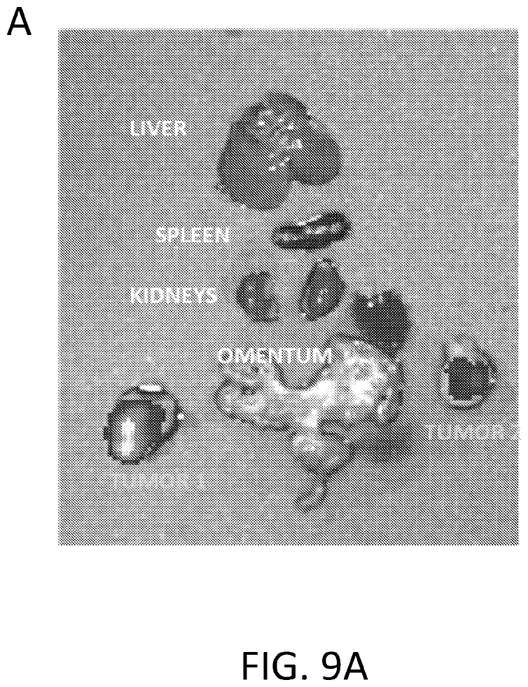

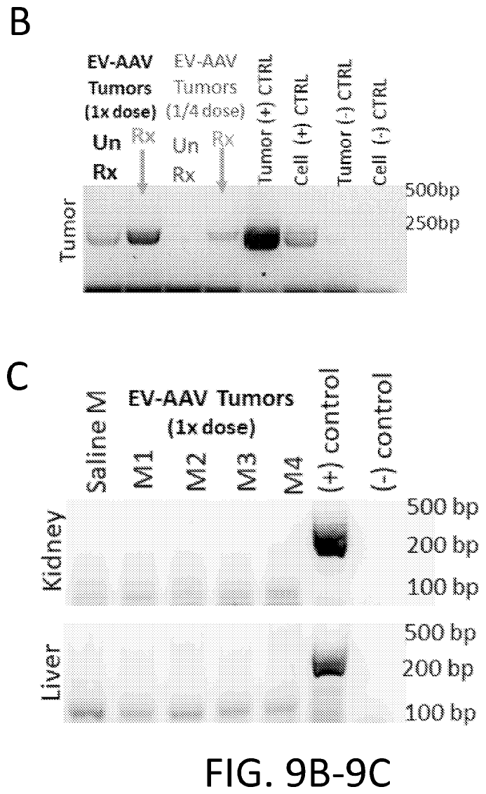

Applicant then evaluated EV distribution in treated mice using 3 independent methods. Tumors (gliomas or sarcomas such as Malignant Peripheral Nerve Sheath Tumors (MPNST)) were injected with fluorescent labeled EVs (DID) and tracked by xenogen IVIS imaging over 48. At 48h mice were sacrificed and organs excised and imaged. The injected L tumor and R tumor exhibited fluorescence detection by IVIS and there was no significant increase above background in the imaged organs (FIG. 8C and FIG. 9).

In independent studies, the inventors loaded EVs with a human cytokine and again injected the left tumor in a biflank model. At 48h animals were sacrificed, organs harvested and processed for human cytokine by Elisa (direct detection) (FIG. 8D). Equivalent cytokine was detectable in the injected and contralateral un-injected tumors. Baseline levels of cytokine was detected in liver or blood. However, IL21 was detected in the kidney above baseline.

As shown in FIG. 9A, the fluorescence signal of DL-EVs was detected only in the 2 tumors and not in the liver, kidneys, spleen or omentum of any of the 2 mice. Quantitative PCR detecting the DNAs loaded to EVs is performed to confirm such finding. These results demonstrate that it is possible to analyze the biodistribution of EVs by direct labeling and the EVs are found to localize within the tumors, although they can get mobilized from the ipsilateral tumor to the contralateral tumor.

To assess if the observed effect was due to renal cell delivery or EV clearance, the inventors repeated the studies with EVs loaded with a gene expression cassette (GFP) in repeated studies involving direct intratumoral (ITu) delivery and systemic IP delivery. Mice were sacrificed after 2 weeks and RNA extracted from the tumors and organs and subjected to PCR using GFP primers. The livers and kidneys were collected from CT2A orthotopic brain tumor mice, treated either intratumorally intracranially (M1,M3) or Intraperitoneally (M2,M4) with EVs carrying enveloped AAV-GFP as cargo at Day 14 post treatment. Total RNA was isolated from these tissues using mini RNA kit (Zymo) and was converted to cDNA followed by PCR amplification using AAV GFP specific primers and separated on a 2% Agarose gel. RNA from Saline treated mouse (IC) was used as negative control and from CT2A cells that stably express GFP were used as positive control.

The results show that the GFP mRNA was expressed in the injected and contralateral uninjected tumor after ITu and that there was a dose effect (less detected transcript with 74 dose (FIG. 9B). In the systemic delivery studies, transcript was undetectable in the Liver or kidney at d14 (by RT PCR, FIG. 9C) but was detected in the tumors of 3 of 4 mice. Taken together these data again suggest the tumor derived EVs preferentially target malignant tumor cells and suggest that the EV associated IL21 cargo detection at 36h represents probable renal clearance rather than cell delivery of cargo at this location.

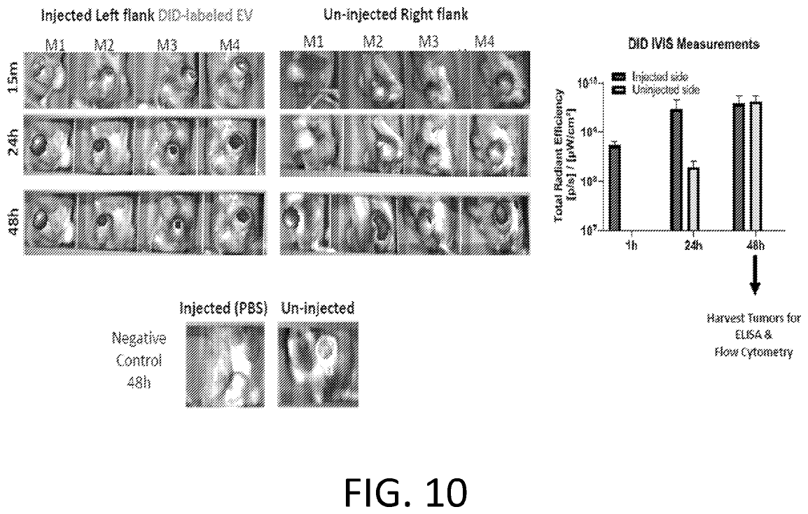

As shown in FIG. 10, fluorescent signal in the left tumor (injected with DiD labeled EVs at one point only) was seen for all the time points. The tumor on the right (injected with saline) showed no DID signal at 15 minutes and 5 hour time points. At 24h post-treatment however, DID detection occurred in the PBS tumor and increased to levels similar to that in the treated side by 48 hour post-treatment. This suggests that the EVs injected in the left tumor are capable of migrating to the uninjected right tumor.

The earlier studies were performed with a single mouse, to determine if this EV migration was reproducible and statistically significant, the study was repeated with 4 mice per cohort. See, FIG. 2. The results confirmed that EVs injected in the left flank migrate to the uninjected contralateral tumor and that by 48h post-treatment the injected and uninjected tumors exhibit equivalent fluorescent signal.

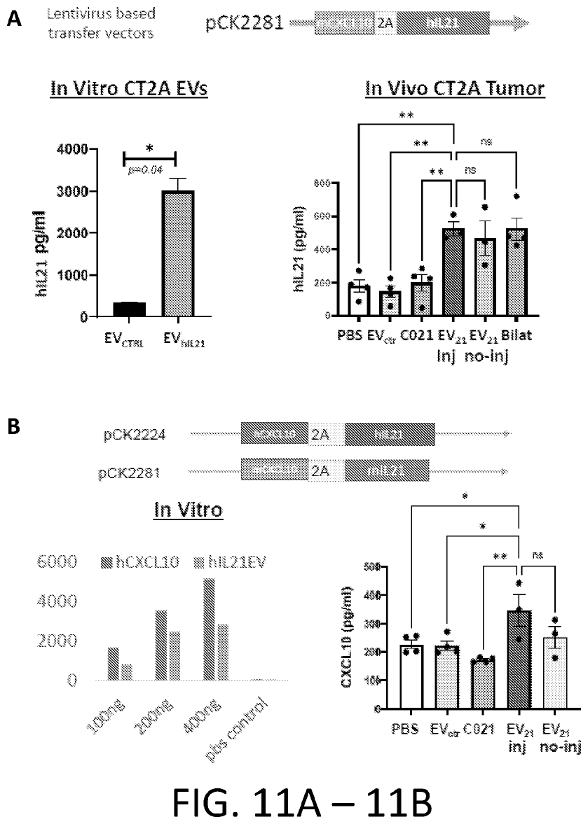

Mice from experiment having its results shown in FIG. 2 were sacrificed at day 2 (following IVIS measurement), and the left and right tumors were isolated and processed separately into single cell suspensions. Supernatant samples from the single cell suspensions were assessed for hIL21 by ELISA. See, FIG. 11. The left panel shows hIL21 expression measurement for the loaded EVs before injecting them in vivo. The graph on the right shows hIL21 detection (400 pg/ml) both in the injected and uninjected tumors (consistent with the DID results above). This provides further corroborative evidence to the IVIS data that the EV_{hCXCL10-IL21} are able to travel and deliver their payload to the uninjected tumor site (FIGS. 11A-11B).

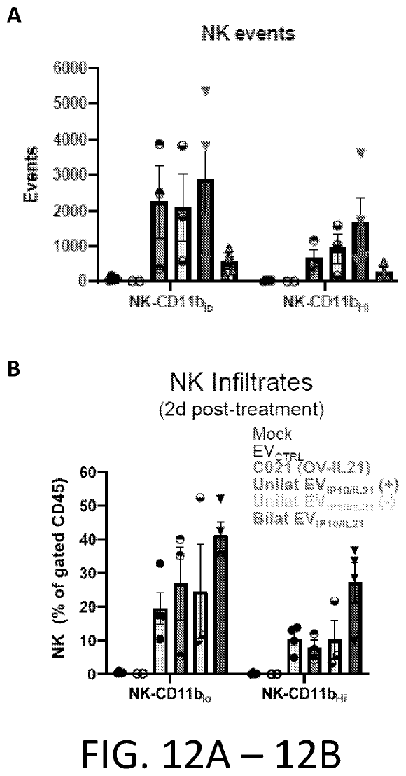

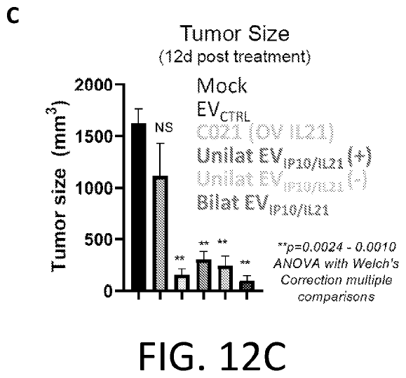

To further explore the mechanistic difference in antitumor efficacy, the immune cell infiltrates at day 2 time point were analyzed. See, FIGS. 12A-12B. The harvested and homogenised mouse tumors were used for immunophenotyping. The number of CD11b high as well as CD11d low NK infiltrates were high in the EV_{hCXCL10-IL21} injected cohorts as compared to the saline group or EV_{CT2A} control group. Amongst the EV_{hCXCL10-IL21} injected cohorts, the NK infiltrate was significantly higher in the bilaterally treated cohort. The immune cell infiltrates at day 6 is under investigation.

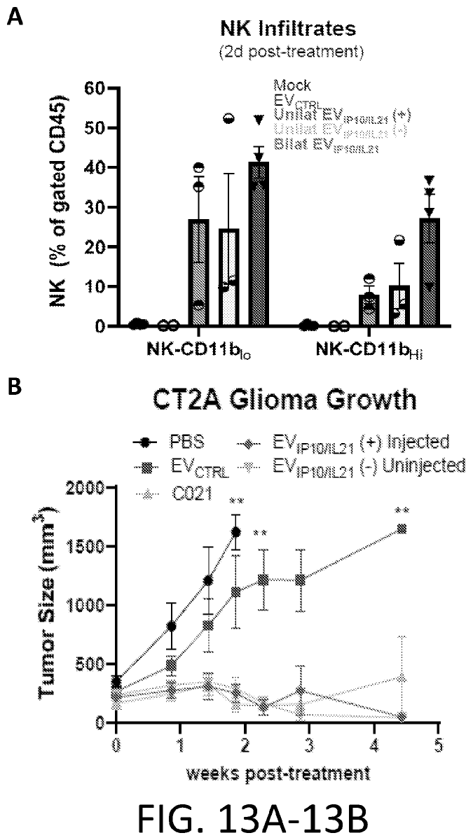

The relative activity of EV_{hCXCL10-IL21} in suppressing tumor growth and improving survival was compared across cohorts of mice treated with unloaded control CT2A EVs or PBS alone. The results are shown in FIGS. 13A-13B. The cohort treated with EV_{hCXCL10-IL21} showed pronounced inhibition of tumor growth as compared with either the control EVs or PBS and complete response (CR) was observed in 100% of the mice within 15 days post treatment. Tumors continued to grow in the control EVs or PBS groups and they met with the tumor burden sacrifice criterion within 15 days of injection and had to be sacrificed.

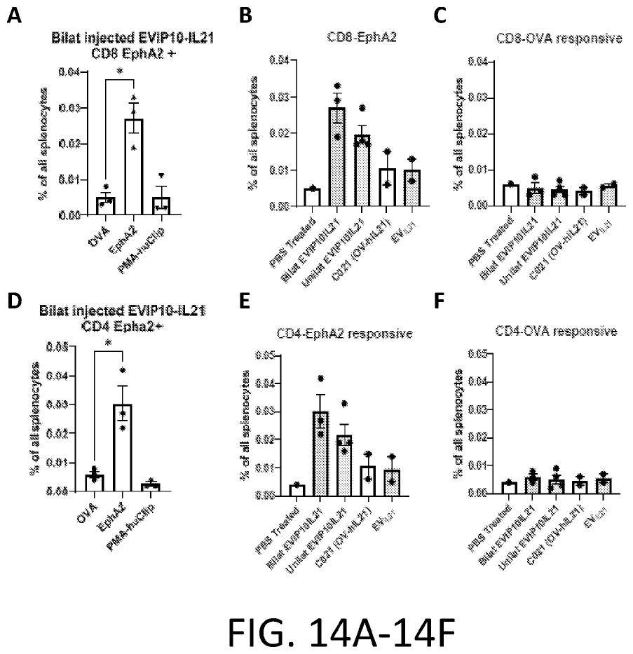

Tumor rechallenge studies were also carried out to assess the development of immunologic memory in the survivor animals from all the cohorts. On day 45, the surviving animals (Complete remission: n=3-4) were rechallenged with tumor, sacrificed 6 days later and their splenocytes isolated and analyzed for tumor reactive T cells (Ephrin A2 tetramer-positive CD4 and CD8⁺ cells) 6 days post rechallenge. The result are shown in FIGS. 14A-14F. The EV_{hCXCL10-IL21}-treated mice exhibited significant EpHA2 antigen-specific CD8⁺ and CD4⁺T cells expansion. There was no significant difference detected between mice treated unilaterally or bilaterally although there was a trend to increased tetramer (+) populations in mice receiving bilateral injections. These responses were specific to the tumor antigen based upon the lack of response to the control antigens (OVA and huCLIP) in these previously inexperienced/unexposed mice.

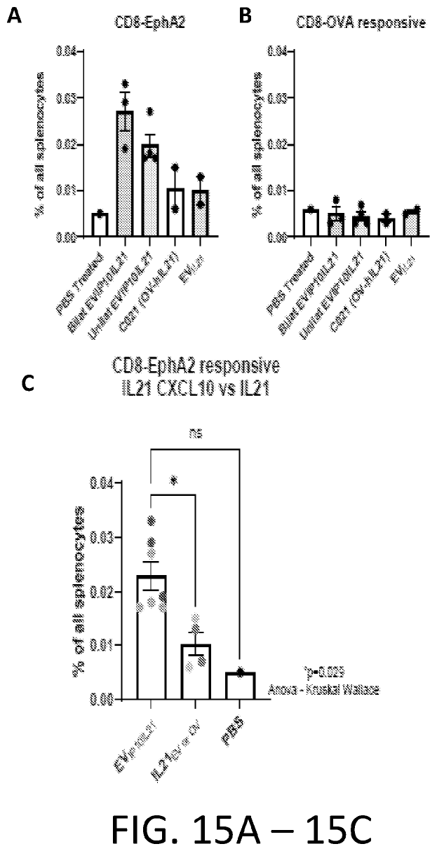

To assess if mice treated with Chemokine+Cytokine combinations had improved T cell memory than those treated with cytokine alone, the tetrameric response was measured within the treated mice. See, FIG. 15. The results show that the mice treated with EV-CXCL10+IL21 (either unilateral or bilateral injection) had improved T cell memory to the tumor antigen than mice treated with bilateral injections of EV-IL21 or C021, an oncolytic virus expressing IL21.

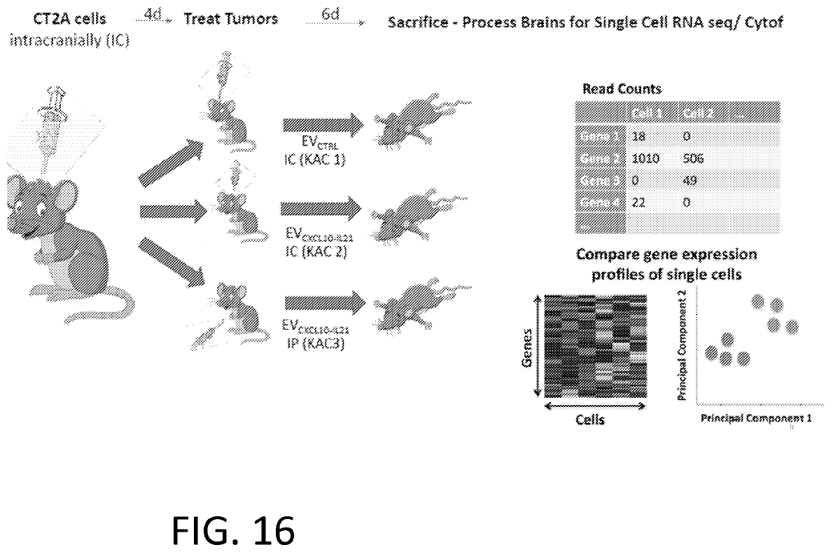

Orthotopic tumors and systemic delivery across the blood brain barrier are under investigation. See, for example, the work flow as illustrated in FIG. 16. The harvested tumors are analyzed by comparing gene expression profiles for single cells (optionally by single cell sequencing).

### Experimental Example No. 4-Gene therapy

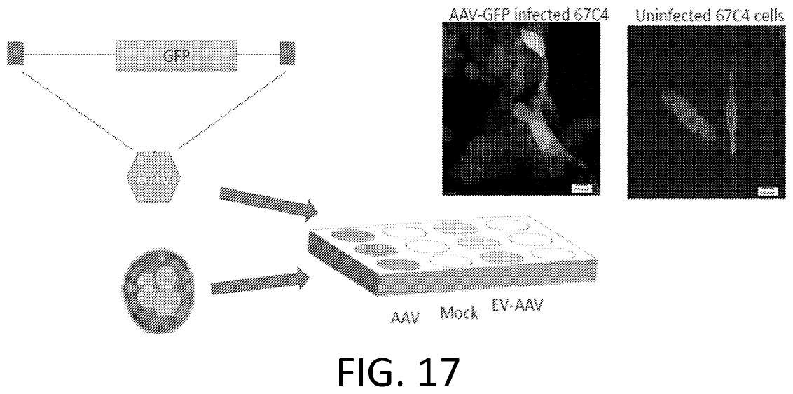

EVs were isolated from 67C4 cells (a murine (B6) MPNST Sarcoma Line) as described previously. Active loading of EVs was achieved by sonication with AAV-GFP viral particles. The sonicated EVs were left at 4° C., overnight and subjected to protease to get rid of any AAV-GFP particles sticking to the surface of EVs. The EVs were then purified further by running through a 300 kD filter and the resultant EVs were used to transfect 67C4 cells. A representative result is shown in FIG. 17. The green color in the cells observed by confocal microscopy indicates the successful delivery of cloaked AAV-GFP virus from EVs to the target cells.

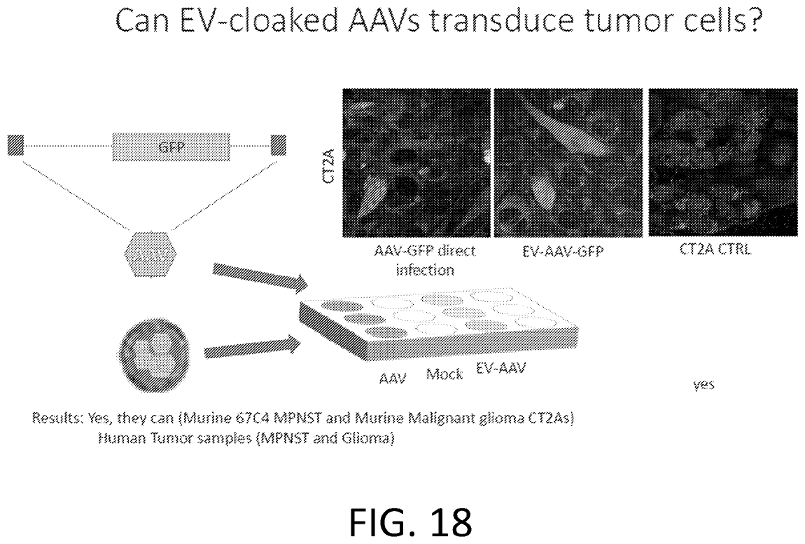

As mentioned above, the CT2A EVs were loaded with AAV-GFP virus and then used to transfect CT2A cells. AAV-GFP virus itself was used as positive control. A representative result is shown in FIG. 18. Bright green GFP expression was seen on cells transfected with EV AAV-GFP which was equivalent to the AAV-GFP direct infection. There was no background green signal on the non treated control CT2A cells.

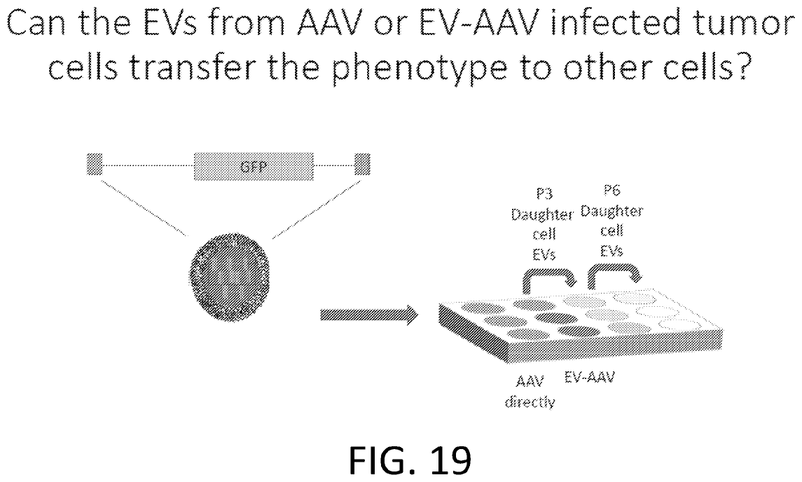

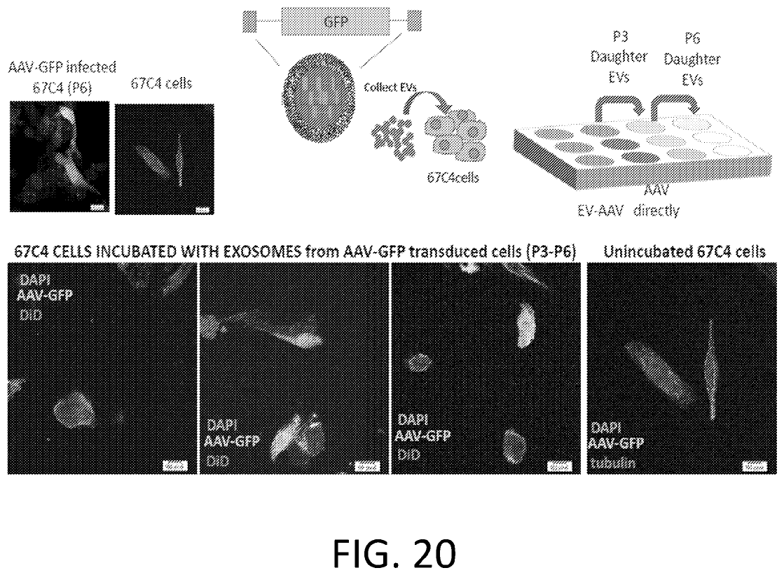

In order to examine the longevity or ability of EVs to continue carrying the payload for generations, the infected cells were passaged for 6 generations, collected and tested for the EVs from all the generations of the 67C4 cells, for example, as illustrated in FIG. 19. A representative result is shown in FIG. 20, showing bright green GFP positive cells even up to generation 6 of the EV transfected cells.

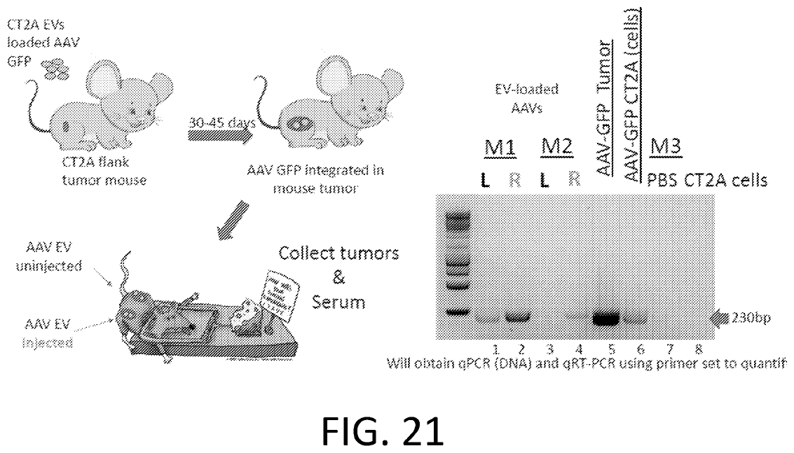

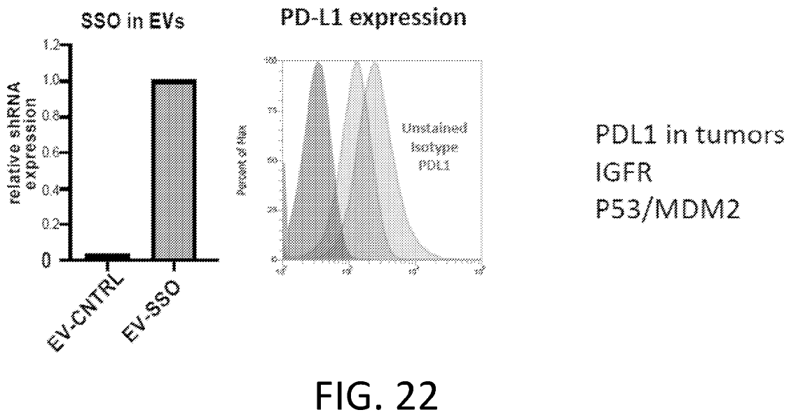

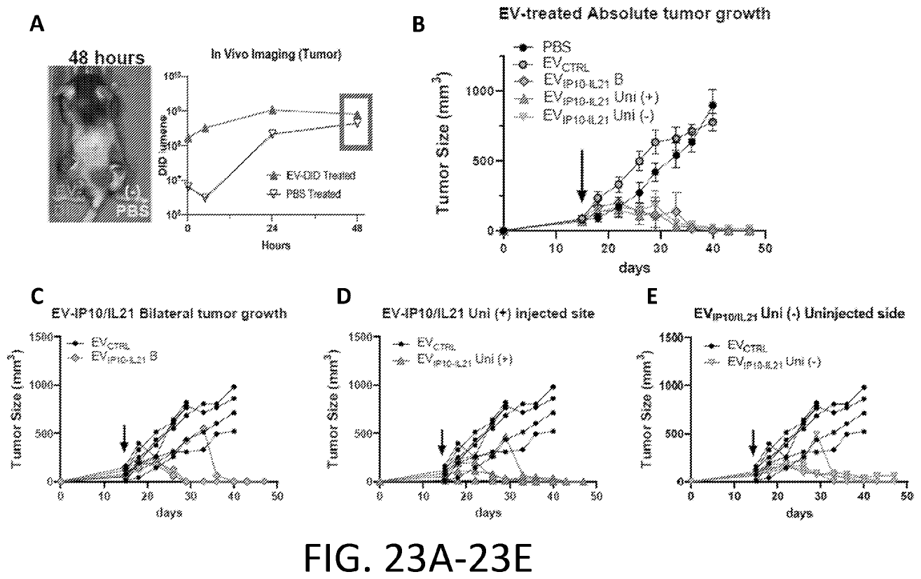

In order to evaluate the efficacy of EVs in delivering the packaged AAV in vivo, the CT2A flank tumor model as described herein was used. Either the AAV-GFP or CT2A EV AAV-GFP was injected intratumorally to the right tumor while the tumor was still 20-50 mm³. The mice were left for 30 days, after which the tumors from both left and right sides were harvested separately. The tumor was divided into halves: one half was formalin fixed for IHC, while the other half was used to extract RNA, converted into cDNA and used for qPCR using GFP specific primers. A representative result is shown in FIG. 21. A 230 bp band specific to GFP was obtained for both the ipsilateral right side and contralateral left side of mouse 1 (M1). This shows that the EVs had delivered the AAV-GFP virus which integrated on both tumors and was detectable by PCR. In mouse 2 (M2), a specific band was observed in the right side only. AAV-GFP virus control in vivo in mouse or in vitro in CT2A also shows a band of similar size and was used as positive control. The PBS treated mouse, mouse 3 (M3), lacks the specific band.

Furthermore, the EVs are loaded with splice-switching oligonucleotides (SSOs) that inhibit MDM2 full-length splicing to increase levels of MDM2-ALT1, p53 levels and functions thereof. Uses of vesicular transports to deliver the SSOs are tested as a methodology to increase levels of MDM2-ALT1 and p53 activity in RMS cell lines and xenografts expressing wildtype p53. The RT qPCR shows that the RH30 (rhabdosarcoma cell line) derived EVs could be successfully loaded using passive loading method.

SSOs for PD-L1 and IGFR are also loaded into EVs and 3 syngeneic lines are constructed.

### EV-Delivered Immunotherapeutics Elicits an Anti-Tumor Response in the Contralateral Tumor.

Method: C57B16 mice bearing Malignant Peripheral Nerve Sheath Tumor (MPNST) tumor 67C4 biflank were treated intratumorally with 67C4 derived EV_{CXCL10-IL21}, once only after the tumor was 50-80 mm cube in size. The tumors were measured using vernier calipers twice a week for tumor growth.

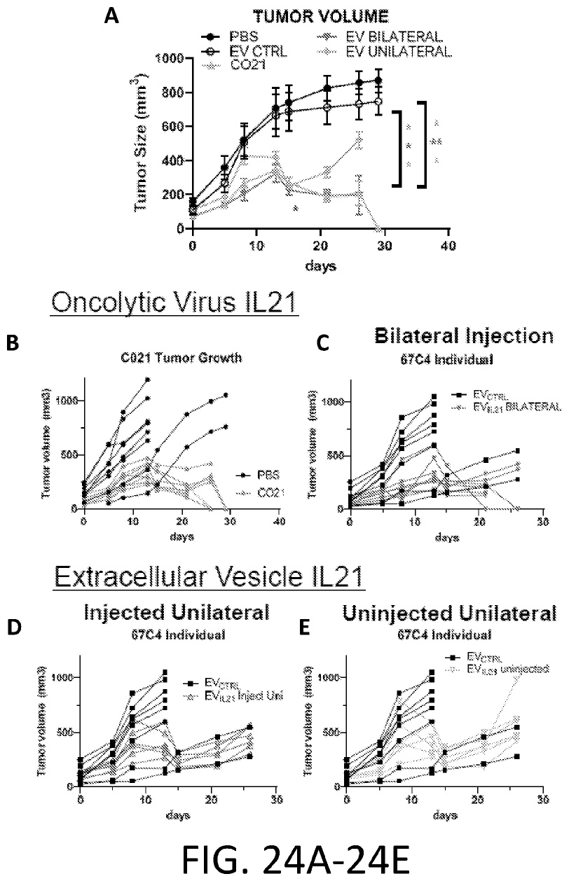

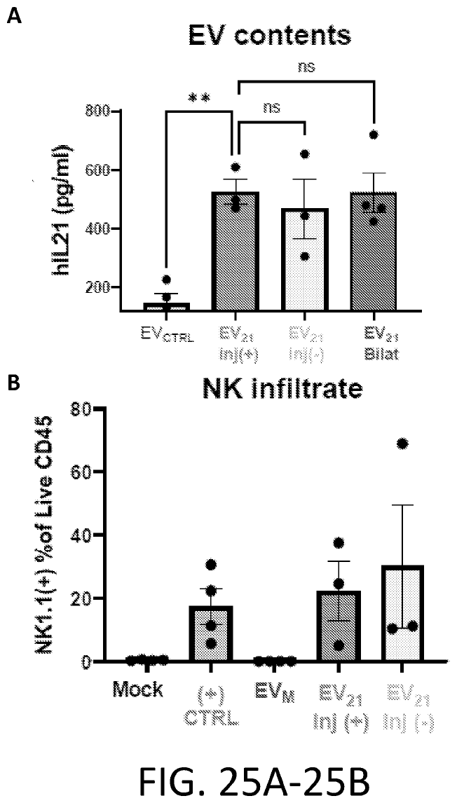

Results: The tumors in control saline and EVCTRL groups kept growing and had to be sacrificed because of the tumor burden. Both uni and bilaterally EV_{hCXCL10-IL21} treated groups were significantly better than both the control groups. The bilateral EV_{hCXCL10-IL21} treatment was more effective than the unilaterally treated group with a marked reduction in tumor growth after an initial increase for around 15 days post treatment. Although, the mice in unilaterally treated group started to grow back slowly around 20 days post treatment, the bilaterally treated group did not regain tumor growth. The results presented here depict that the EV_{hCXCL10-IL21} treatment is capable to restricting tumor growth in Malignant Peripheral Nerve Sheath Tumor (MPNST) as well. Further, Applicant has only tested 1 dose of EV_{hCXCL10-IL21} for consistency, but a higher dose or repeated dosing may be able to eliminate the tumors completely in this model, too. See FIGS. 24A-24E.

### Cytokines Improve Oncolytic Virus Response in CT2A Brain Tumors.

Method: Glioma (mouse) cell line CT2A was injected stereotactically in C57BL6 mice using a stereotactic frame. 7 days post implantation, the mice were treated with saline or an oncolytic herpes simplex virus (oHSV) expressing hIL21 (left panel), or oHSV expressing mouse CXCL10 and IL21 (CO25) intracranially. C134 and C154 treated mice served as controls. The mice were weighed 3 times per week and monitored for focal neurologic deficits, neurologic changes and weight loss.

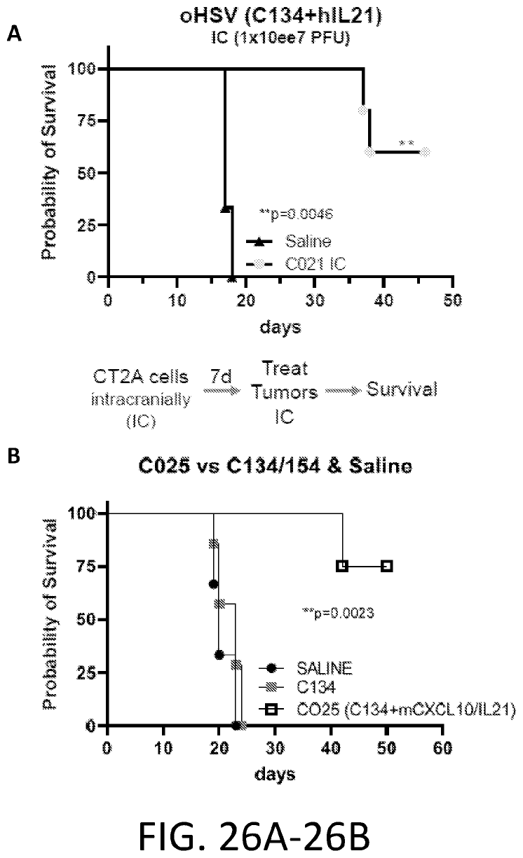

Results: The control mice succumbed within first 20 days post tumor implantation. In the CO25 (mIL21+mCXCL10) treated mice ¾ of the mice survived. In the CO21 treated mice (hIL21) 60% of mice survived 50 days post implantation. These results and the flank based studies suggested that IL21 provides a therapeutic benefit and reduced tumor growth. Applicant therefore used the OV-IL21 virus as a positive control for efficacy of oHSV delivered cytokines in vivo. See, FIGS. 26A-26B.

### EVs Provide a Therapeutic Response Across the Blood Brain Barrier

Method: Mice bearing orthotropic CT2A gliomas (method as in Slide 23) were treated with Saline (negative control) or C021 (+control) by intracranial injection using the same stereotactic coordinates used for tumor implantation. Three experimental cohorts were treated with intracranial (IC) or Intraperitoneal delivery of EV CXCL10-IL21 at (1) 250 pg IC, (2) 500 pg IC, or (3) 500 pg and followed for survival.

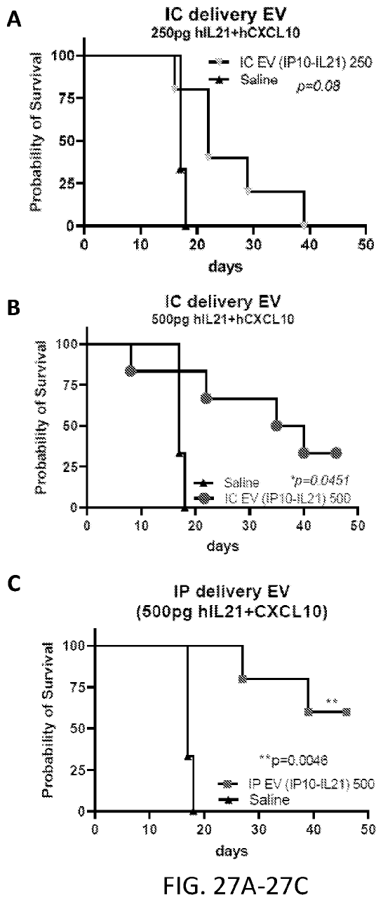

Results: IC delivery of EV CXCL10-IL21 showed a dose response effect. The cohort treated with low dose (250 pg IC) did not survive beyond 40 days post tumor implantation. While there was some difference in the median survival in the IC 250 vs saline group (22d vs 17d) it approached but did not achieve statistical significance by Log Rank analysis (IC250 vs Saline, p=0.08). When mice were treated with a higher EV dose (500 pg of CXCL10-IL21 by EV) this significantly improved median survival (37.5d vs 17d: p=0.045 Log Rank test) over saline treated mice and ¼ of the mice cleared their tumors. The final cohort treated with 500 pg of CXCL10 and IL21 but by IP delivery did the best with the majority of mice surviving long term (60% survival at 50 day time point with single treatment **p=0.0046 log Rank). See, FIGS. 27A-27C. In summary, these results show that there was a dose response effect in the IC treated mice but that the IP delivered EV_{hCXCL10-IL21} was most effective and in terms of median survival and overall survival advantage.

### Therapeutic EVs Traffic to CNS Tumors

Method: Orthotopic CT2A tumor mice were treated either IC or IP as above. Brains harvested from the mice sacrificed at D6 post treatment were used for flow cytometric analyses.

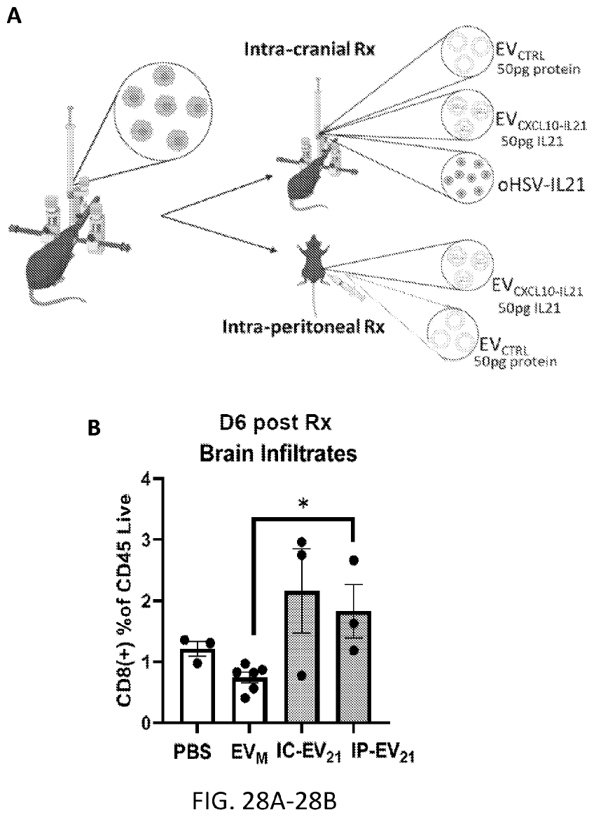

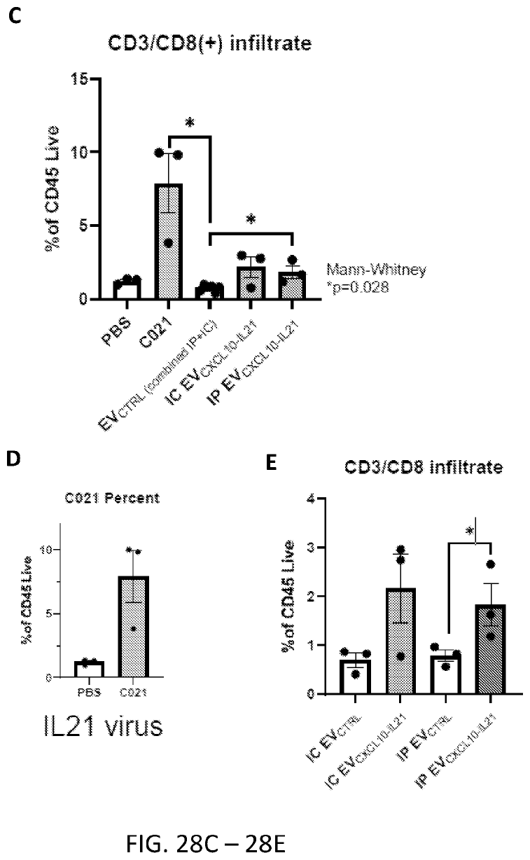

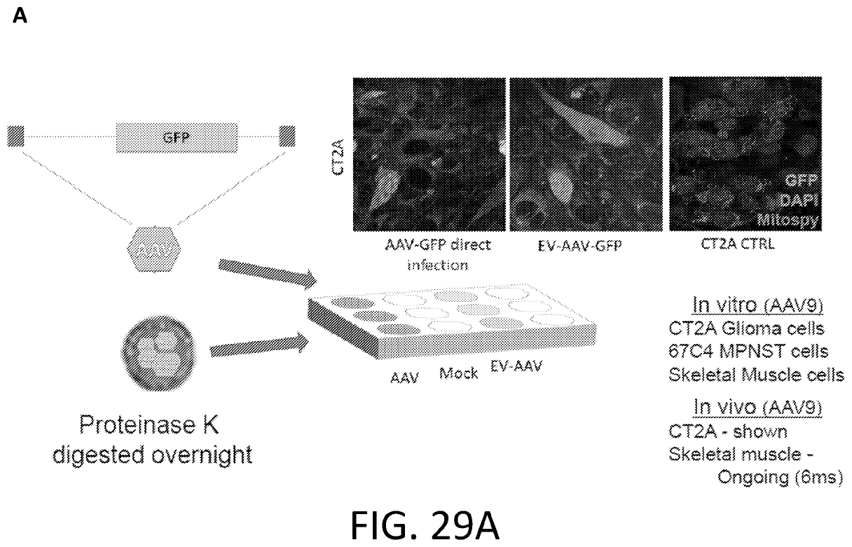

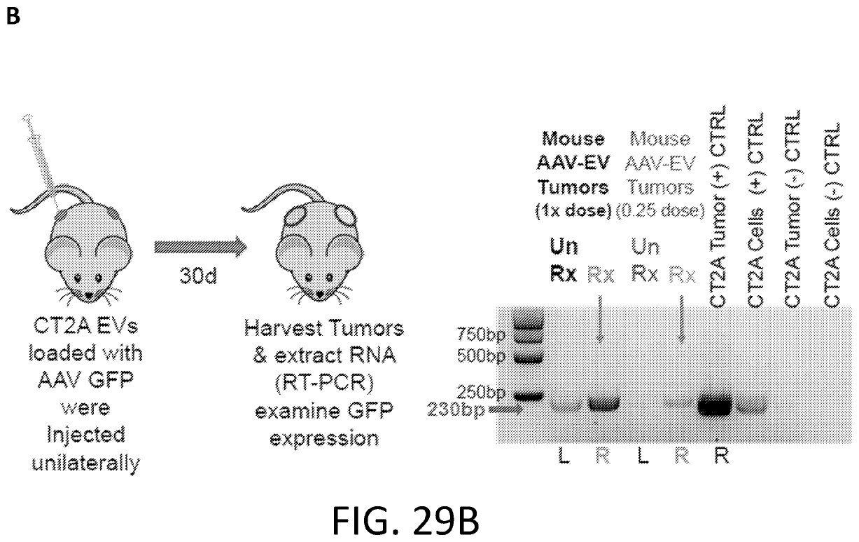

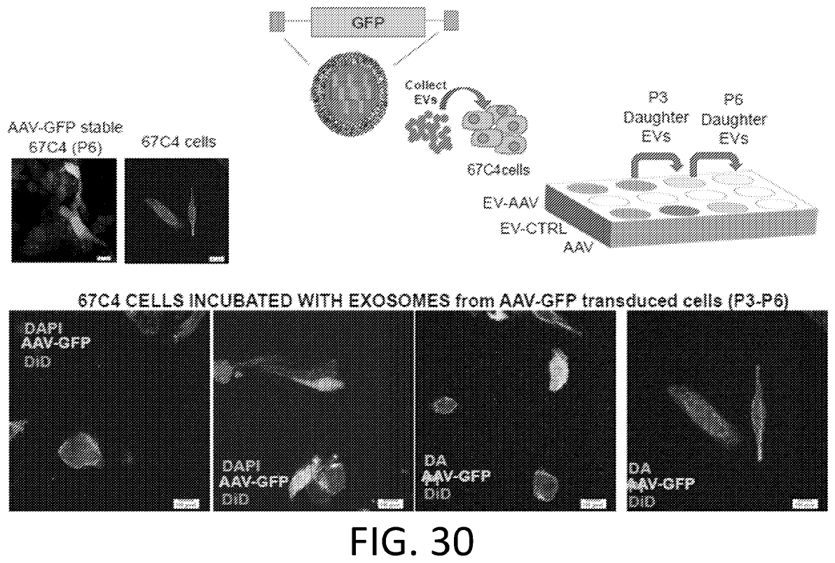

Results: The mice treated with EV_{hCXCL10-IL21} had significantly higher CD45CD8+ cells as compared to saline or unloaded EV control treated mice (combined IP and IC EVCTRL treated mice). See. FIGS. 28A-28B. FIGS. 28C-28E are different representations of this same experiment but with representative Flow cytometry panels shown for the treatment cohorts and also includes the CD8 influx for C021 treated mice (FIG. 28C). Separate analysis of C021 vs Saline (FIG. 28D) and comparison of flow plots from IP and IC EV treatment cohorts (FIG. 28E).

scRNAseq Analyses Suggest EVs Target a Cell Cluster with Markers Enriched for Malignant Glioma Progression, Mesenchymal Transition and Poor Prognosis.

Single cell RNA sequence (scRNAseq) studies show that IP-delivered EVs (carrying a GFP minigene expression cassette) preferentially target a cell cluster (Cluster 3) with gene expression enriched for malignant glioma progression, mesenchymal transition and poor prognosis markers (e.g., BCAS1, Sox10, Sox6, Opalin, ERMN, MOBP, PLP1) and not to Macrophage/Monocytic cells (e.g., CD45, CD11b, CD14, and/or C1qa positive cells) in the brain. These same mice are represented in FIGS. 9A-9C which show that the GFP cassette was detected in the brain but was not detected by RT-PCR from liver or kidney. Independent flank based studies also showed that the GFP expression occurs in flank tumors (FIGS. 9A-9C) and here in brain tumors and in these single cell RNAseq studies.

### List of Sequences of the Disclosure

The hIL21 nucleotide (NT) sequence:

(SEQ ID NO: 1)
ATGAGATCCAGTCCTGGCAACATGGAGAGGATTGTCATCTGTCTG
ATGGTCATCTTCTTGGGGACACTGGTCCACAAATCAAGCTCCCAA
GGTCAAGATCGCCACATGATTAGAATGCGTCAACTTATAGATATT
GTTGATCAGCTGAAAAATTATGTGAATGACTTGGTCCCTGAATTT
CTGCCAGCTCCAGAAGATGTAGAGACAAACTGTGAGTGGTCAGCT
TTTTCCTGCTTTCAGAAGGCCCAACTAAAGTCAGCAAATACAGGA
AACAATGAAAGGATAATCAATGTATCAATTAAAAAGCTGAAGAGG
AAACCACCTTCCACAAATGCAGGGAGAAGACAGAAACACAGACTA
ACATGCCCTTCATGTGATTCTTATGAGAAAAAACCACCCAAAGAA
TTCCTAGAAAGATTCAAATCACTTCTCCAAAAGATGATTCATCAG
CATCTGTCCTCTAGAACACACGGAAGTGAAGATTCCTAA

### Hil21 Aa Sequence:

(SEQ ID NO: 2)
MRSSPGNMERIVICLMVIFLGTLVHKSSSQGQDRHMIRMRQLIDI
VDQLKNYVNDLVPEFLPAPEDVETNCEWSAFSCFQKAQLKSANTG
NNERIINVSIKKLKRKPPSTNAGRRQKHRLTCPSCDSYEKKPPKE
FLERFKSLLQKMIHQHLSSRTHGSEDS*

### 3. Hcxcl10 Nt Sequence:

(SEQ ID NO: 3)
ATGAATCAAACTGCCATTCTGATTTGCTGCCTTATCTTTCTGACT
CTAAGTGGCATTCAAGGAGTACCTCTCTCTAGAACTGTACGCTGT
ACCTGCATCAGCATTAGTAATCAACCTGTTAATCCAAGGTCTTTA
GAAAAACTTGAAATTATTCCTGCAAGCCAATTTTGTCCACGTGTT
GAGATCATTGCTACAATGAAAAAGAAGGGTGAGAAGAGATGTCTG
AATCCAGAATCGAAGGCCATCAAGAATTTACTGAAAGCAGTTAGC
AAGGAAAGGTCTAAAAGATCTCCCTAA

### Hcxcl10 Aa Sequence:

(SEQ ID NO: 4)
MNQTAILICCLIFLTLSGIQGVPLSRTVRCTCISISNQPVNPRSL
EKLEIIPASQFCPRVEIIATMKKKGEKRCLNPESKAIKNLLKAVS
KERSKRSP*

The 2A Element (2A is not translated but allows the ribosome to slip through and then express the second sequence):

(SEQ ID NO: 5)
TCGCGAGCTAAGAGGGAGGGCAGAGGGAGCCTGCTCACCTGCGGA
GACGTGGAAGAGAATCCCGGGCCT

hCXCL10-2A-hIL21 NT Sequence

(SEQ ID NO: 6)
ATGAATCAAACTGCCATTCTGATTTGCTGCCTTATCTTTCTGACT
CTAAGTGGCATTCAAGGAGTACCTCTCTCTAGAACTGTACGCTGT
ACCTGCATCAGCATTAGTAATCAACCTGTTAATCCAAGGTCTTTA
GAAAAACTTGAAATTATTCCTGCAAGCCAATTTTGTCCACGTGTT
GAGATCATTGCTACAATGAAAAAGAAGGGTGAGAAGAGATGTCTG
AATCCAGAATCGAAGGCCATCAAGAATTTACTGAAAGCAGTTAGC
AAGGAAAGGTCTAAAAGATCTCCCTCGCGAGCTAAGAGGGAGGGC
AGAGGGAGCCTGCTCACCTGCGGAGACGTGGAAGAGAATCCCGGG
CCTATGAGATCCAGTCCTGGCAACATGGAGAGGATTGTCATCTGT
CTGATGGTCATCTTCTTGGGGACACTGGTCCACAAATCAAGCTCC
CAAGGTCAAGATCGCCACATGATTAGAATGCGTCAACTTATAGAT
ATTGTTGATCAGCTGAAAAATTATGTGAATGACTTGGTCCCTGAA
TTTCTGCCAGCTCCAGAAGATGTAGAGACAAACTGTGAGTGGTCA
GCTTTTTCCTGCTTTCAGAAGGCCCAACTAAAGTCAGCAAATACA
GGAAACAATGAAAGGATAATCAATGTATCAATTAAAAAGCTGAAG
AGGAAACCACCTTCCACAAATGCAGGGAGAAGACAGAAACACAGA
CTAACATGCCCTTCATGTGATTCTTATGAGAAAAAACCACCCAAA
GAATTCCTAGAAAGATTCAAATCACTTCTCCAAAAGATGATTCAT
CAGCATCTGTCCTCTAGAACACACGGAAGTGAAGATTCCTAA

hCXCL10-2A-hIL21 Amino Acid Sequence

(SEQ ID NO: 7)
MNQTAILICCLIFLTLSGIQGVPLSRTVRCTCISISNQPVNPRSL
EKLEIIPASQFCPRVEIIATMKKKGEKRCLNPESKAIKNLLKAVS
KERSKRSPSRAKREGRGSLLTCGDVEENPGPMRSSPGNMERIVIC
LMVIFLGTLVHKSSSQGQDRHMIRMRQLIDIVDQLKNYVNDLVPE
FLPAPEDVETNCEWSAFSCFQKAQLKSANTGNNERIINVSIKKLK
RKPPSTNAGRRQKHRLTCPSCDSYEKKPPKEFLERFKSLLQKMIH
QHLSSRTHGSEDS

### Sample SSO Sequences

| Gene | Target | SSO Sequence |
|---|---|---|
| Product | Sequence | (antisense) |
| INSR Intron | GTGTCAGAG | ACCACTGG |
| 10-11 | CCCAGTGGT | GCTCTGACAC |
| CUGBP1 | (SEQ ID NO: 8) | (SEQ ID NO: 9) |
| Non- | CGAGCGTGA | TTAGTTTAA |
| specific | TTAAACTAA | TCACGCTCG |
| | (SEQ ID NO: 10) | (SEQ ID NO: 11) |
| MDM2 | AAGAAGATCC | GAAATTTCA |
| | TGAAATTTC | GGATCTTCTT |
| | (SEQ ID NO: 12) | (SEQ ID NO: 13) |
| PD-L1 SS01 | TTCTGGAATGC | ATAATTAGGG |
| | CCTAATTAT | CATTCCAGAA |
| | (SEQ ID NO: 14) | (SEQ ID NO: 15) |
| PD-L1 SS02 | ATTTGGTGTC | TGAACGGTA |
| | TTACCGTTCA | AGACACCAAAT |
| | (SEQ ID NO: 16) | (SEQ ID NO: 17) |

The primers used to construct the PLK-based expression Lentivirus-(Plasmid homology regions for strand displacement ligation are in bold and the SSO target sequence are not in bold):

| | |
|---|---|
| MDM2 (SSO #12, | AGGACGAAACACCGGGAAATTTCAGGATCTTCTTC |
| Chandler) Infusion Fwd | TTTTTGAATTCTCGACCTCGA (SEQ ID NO: 18) |
| MDM2 (SSO #12, | TCGAGGTCGAGAATTCAAAAAGAAGAAGATCCTG |
| Chandler) Infusion Rev | AAATTTCCCGGTGTTTCGTCCT (SEQ ID NO: 19) |
| MDM2 (SSO control | AGGACGAAACACCGGTTAGTTTAATCACGCTCGTT |
| Chandler) Infusion Fwd | TTTGAATTCTCGACCTCGA (SEQ ID NO: 20) |
| MDM2 (SSO control | TCGAGGTCGAGAATTCAAAAACGAGCGTGATTAA |
| Chandler) Infusion Rev | ACTAACCGGTGTTTCGTCCT (SEQ ID NO: 21) |
| INSR pLenti ASO55F | AGGACGAAACACCGGACCACTGGGCTCTGACACT |
| | TTTTTGAATTCTCGACTCGA (SEQ ID NO: 22) |
| INSR pLenti AsO55R | TCGAGGTCGAGAATTCAAAAAAGTGTCAGAGCCC |
| | AGTGGTCCGGTGTTTCGTCCT (SEQ ID NO: 23) |
| ASO1: PD-L1 Exon 2 5′ | AGGACGAAACACCGGTTCTGGAATGCCCTAATTAT |
| ASOF | TTTTTTGAATTCTCGACTCGA (SEQ ID NO: 24) |
| ASO 2: PD-L1 Exon 2 | TCGAGGTCGAGAATTCAAAAAAATTTGGTGTCTTA |
| 3′ ASOF | CCGTTCACCGGTGTTTCGTCCT (SEQ ID NO: 25) |

### EQUIVALENTS

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

All publications, patent applications, patents, and other references mentioned herein are expressly incorporated by reference in their entirety, including all formulas and figures, to the same extent as if each were incorporated by reference individually. In case of conflict, the present specification, including definitions, will control.

Other embodiments are set forth within the following claims.

What is claimed is:

1. A method for one or more of:
(a) treating a cancer in a subject in need thereof, or
(b) conferring or inducing an immune response to a cancer in a subject in need thereof,
the method comprising administering to the subject an effective amount of extracellular vesicles (EVs) isolated from cancer cells of the subject, and wherein the EVs comprise a therapeutic agent.

2. The method of claim 1, wherein the EVs further comprise a surface membrane associated protein that selectively targets a cancer cell.

3. The method of claim 1 or 2, wherein the isolated cancer cells are engineered to produce the therapeutic agent in the EVs.

4. The method of any one of claims 1-3, wherein the therapeutic agent is a therapeutic polynucleotide.

5. The method of claim 4, wherein the therapeutic polynucleotide comprises or expresses a splice-switching oligonucleotide (SSO) or an antisense polynucleotide, and wherein the SSO or antisense polynucleotide reduces the expression of an oncogene or an immune suppressor in a cell of the cancer to be treated.

6. The method of claim 5, wherein the oncogene comprises one or more of: Insulin Like Growth Factor 1 Receptor (IGFR), Tumor Protein P53 (p53), or MDM2 Proto-Oncogene (MDM2).

7. The method of claim 5, wherein the immune suppressor comprises PD-L1.

8. The method of claim 4, wherein the therapeutic polynucleotide expresses a protein toxin or an immune activator.

9. The method of any one of claims 4 to 8, wherein the EVs comprise a vector comprising the polynucleotide.

10. The method of claim 9, wherein the vector is selected from a plasmid, a retroviral vector, a lentiviral vector, a non-replicating lentiviral vector, an adenovirus vector, or an adeno-associated virus vector.

11. The method of any one of claims 1 to 10, wherein the therapeutic agent comprises a toxin, or an immune activator.

12. The method of claim 11, wherein the toxin comprises one or more of: a chemotherapeutic agent, a radiopharmaceutical, or a protein toxin.

13. The method of claim 12, wherein the chemotherapeutic agent comprises one or more of: an alkylating agent, a nitrosourea, an antimetabolite, an anti-tumor antibiotic, a topoisomerase inhibitor, a mitotic inhibitor, or a corticosteroid.

14. The method of any one of claims 8 to 11, wherein the immune activator comprises a cytokine, an immune checkpoint inhibitor, or a neoantigen target directing an immune component to the cancer cells.

15. The method of claim 14, wherein the cytokine comprises one or more of: Interleukin 12 (IL-12), Interleukin 18 (IL-18), Interleukin 15 (IL-15), Interleukin 21 (IL-21), Interleukin 27 (IL-27), C—X—C Motif Chemokine Ligand 9 (CXCL9), C—X—C Motif Chemokine Ligand 10 (CXCL10), C—X—C Motif Chemokine Ligand 11 (CXCL11), Interferon α (IFNα), Type I interferon (IFNI), Type II interferon (IFNII), Interferon γ (IFNγ), Interleukin 2 (IL-2), Interleukin 5 (IL-5), Tumor Necrosis Factor (TNF), Interferon β (IFNβ), Interleukin 18 (IL-18), CD40 Ligand (CD40L), Colony Stimulating Factor 2 (CSF2), or an equivalent of each thereof.

16. The method of claim 14 or 15, wherein the cytokine comprises CXCL10 and IL-12.

17. The method of claim 14 or 15, wherein the cytokine comprises CXCL10 and IL-21.

18. The method of claim 14 or 15, wherein the cytokine comprises CXCL10 and IL-27.

19. The method of claim 14, wherein the neoantigen comprises one or more of: an embryonic antigen optionally Ephrin Type-A Receptor 2 (EphA2), Interleukin 13 Receptor Subunit Alpha 2 (IL13Ra2) homodimers, carcinoembryonic antigen (CEA), or heat shock protein (hsp) gp96 (HSP96); alphafetoprotein (AFP), CA-125 (Mucin 16, Cell Surface Associated), MUC-1 (Mucin 1, Cell Surface Associated), Epithelial tumor antigen (ETA), Tyrosinase, Melanoma-associated antigen (MAGE), abnormal products of ras, p53, CD10, CD19, CD20, CD21, CD22, CD25, CD30, CD33, CD34, CD37, CD44v6, CD45, CDw52, Fms-like tyrosine kinase 3 (FLT-3, CD135), c-Kit (CD117), CSF1R (Colony Stimulating Factor 1 Receptor, CD115), CD133, PDGFR-α (CD140a), PDGFR-β (CD 140b), chondroitin sulfate proteoglycan 4 (CSPG4, melanoma-associated chondroitin sulfate proteoglycan), EGFR (Epidermal Growth Factor Receptor), de2-7-EGFR (EGFRvIII), Folate binding protein, Her2neu, Her3, PSMA (Prostate-Specific Membrane Antigen), PSCA (Prostate Stem Cell Antigen), PSA (Prostate-Specific Antigen), TAG-72, HLA-DR, IGFR, IL3R, fibroblast activating protein (FAP), Carboanhydrase IX (MN/CA IX), Carcinoembryonic antigen (CEA), EpCAM, CDCP1, Derlin1, Tenascin, frizzled 1-10, VEGFR2 (KDR/FLK1), VEGFR3 (FLT4, CD309), Endoglin, CLEC14, Tem1-8, or Tie2.

20. The method of claim 14 or 19, wherein the immune component comprises an immune cell specifically recognizing and binding to the neoantigen, an antibody specifically recognizing and binding to the neoantigen, or both.

21. The method of claim 20, wherein the immune cell comprises one or more of: an NK cell, an NKT cell, a T cell, a B cell, a dendritic cell, a splenocyte or a macrophage.

22. The method of claim 20 or 21, wherein the immune cell expressing a chimeric antigen receptor (CAR) specifically recognizing and binding to the neoantigen.

23. The method of claim 20, wherein the antibody is an immune cell engager specifically recognizing and binding to the neoantigen and a cell surface marker of the immune cell.

24. The method of claim 23, wherein the cell surface marker of the immune cell comprises one or more of CD3, CD28, 4-1BB, CD16, NKG2D or CD64.

25. The method of any one of claims 1 to 24, wherein the isolated cancer cells are from a tumor biopsy of the subject.

26. The method of any one of claims 1 to 25, further comprising resection of the cancer in the subject prior to the administration.

27. The method of any one of claims 1 to 26, wherein the administration is systemically or locally to a tumor in the subject.

28. The method of any one of claims 1 to 27, wherein the cancer is primary, metastatic or relapsed.

29. The method of any one of claims 1 to 28, wherein the cancer to be treated comprises a solid tumor, optionally a brain tumor, and further optionally a glioma.

30. The method of any one of claims 1 to 29, wherein the method is selected from a first line therapy, a second line therapy, a third line therapy, a fourth line therapy or a fifth line therapy.

31. The method of any one of claims 1 to 30, wherein the administration is repeated.

32. The method of any one of claims 1 to 31, further comprising administering an anti-cancer therapy to the subject.

33. The method of claim 32, wherein the anti-cancer therapy comprises one or more of: a surgical resection of the cancer, a radiation therapy, a chemotherapy, or an immunotherapy.

34. The method of any one of claims 1 to 33, wherein the subject is a mammal.

35. The method of claim 34, wherein the mammal is selected from a canine, a feline, an equine, or a human patient.

36. The method of any one of claims 1 to 35, wherein the cancer is selected from a carcinoma or a sarcoma.

37. An isolated extracellular vesicle (EV) isolated from a cancer cell, wherein the EV comprises a therapeutic agent.

38. The isolated EV of claim 37, wherein the EVs further comprise a surface membrane associated protein that selectively targets a cancer cell.

39. The isolated EV of claim 37 or 38, wherein the cancer cells are engineered to produce the therapeutic agent in the EVs.

40. The isolated EV of any one of claims 37 to 39, wherein the therapeutic agent is a therapeutic polynucleotide.

41. The isolated EV of claim 40, wherein the therapeutic polynucleotide comprises or expresses a splice-switching oligonucleotide (SSO) or an antisense polynucleotide, and wherein the SSO or antisense polynucleotide reduces the expression of an oncogene or an immune suppressor in a cell of the cancer to be treated.

42. The isolated EV of claim 41, wherein the oncogene comprises one or more of: Insulin Like Growth Factor 1 Receptor (IGFR), Tumor Protein P53 (p53), or MDM2 Proto-Oncogene (MDM2).

43. The isolated EV of claim 41, wherein the immune suppressor comprises PD-L1.

44. The isolated EV of claim 40, wherein the therapeutic polynucleotide expresses a protein toxin or an immune activator.

45. The isolated EV of any one of claims 40 to 44, comprising a vector that comprises the polynucleotide.

46. The isolated EV of claim 45, wherein the vector is selected from a plasmid, a retroviral vector, a lentiviral vector, a non-replicating lentiviral vector, an adenovirus vector, or an adeno-associated virus vector.

47. The isolated EV of any of claims 37 to 46, wherein the therapeutic agent comprises a toxin, or an immune activator.

48. The isolated EV of claim 47, wherein the toxin comprises one or more of: a chemotherapeutic agent, a radiopharmaceutical, or a protein.

49. The isolated EV of claim 48, wherein the chemotherapeutic agent comprises one or more of: an alkylating agent, a nitrosourea, an antimetabolite, an anti-tumor antibiotic, a topoisomerase inhibitor, a mitotic inhibitor, or a corticosteroid.

50. The isolated EV of claim 47, wherein the immune activator comprises a cytokine, an immune checkpoint inhibitor, or a neoantigen target directing an immune component to the cancer cells.

51. The isolated EV of claim 50, wherein the cytokine comprises one or more of: Interleukin 12 (IL-12), Interleukin 18 (IL-18), Interleukin 15 (IL-15), Interleukin 21 (IL-21), Interleukin 27 (IL-27), C—X—C Motif Chemokine Ligand 9 (CXCL9), C—X—C Motif Chemokine Ligand 10 (CXCL10), C—X—C Motif Chemokine Ligand 11 (CXCL11), Interferon α (IFNα), Type I interferon (IFNI), Type II interferon (IFNII), Interferon γ (IFNγ), Interleukin 2 (IL-2), Interleukin 5 (IL-5), Tumor Necrosis Factor (TNF), Interferon β (IFNβ), Interleukin 18 (IL-18), CD40 Ligand (CD40L), Colony Stimulating Factor 2 (CSF2), or an equivalent of each thereof.

52. The isolated EV of claim 50 or 51, wherein the cytokine comprises CXCL10 and IL-12.

53. The isolated EV of claim 50 or 51, wherein the cytokine comprises CXCL10 and IL-21.

54. The isolated EV of claim 50 or 51, wherein the cytokine comprises CXCL10 and IL-27.

55. The isolated EV of claim 50, wherein the neoantigen comprises one or more of: an embryonic antigen optionally Ephrin Type-A Receptor 2 (EphA2), Interleukin 13 Receptor Subunit Alpha 2 (IL13Ra2) homodimers, carcinoembryonic antigen (CEA), or heat shock protein (hsp) gp96 (HSP96); alphafetoprotein (AFP), CA-125 (Mucin 16, Cell Surface Associated), MUC-1 (Mucin 1, Cell Surface Associated), Epithelial tumor antigen (ETA), Tyrosinase, Melanoma-associated antigen (MAGE), abnormal products of ras, p53, CD10, CD19, CD20, CD21, CD22, CD25, CD30, CD33, CD34, CD37, CD44v6, CD45, CDw52, Fms-like tyrosine kinase 3 (FLT-3, CD135), c-Kit (CD117), CSF1R (Colony Stimulating Factor 1 Receptor, CD115), CD133, PDGFR-α (CD140a), PDGFR-β (CD 140b), chondroitin sulfate proteoglycan 4 (CSPG4, melanoma-associated chondroitin sulfate proteoglycan), EGFR (Epidermal Growth Factor Receptor), de2-7-EGFR (EGFRvIII), Folate binding protein, Her2neu, Her3, PSMA (Prostate-Specific Membrane Antigen), PSCA (Prostate Stem Cell Antigen), PSA (Prostate-Specific Antigen), TAG-72, HLA-DR, IGFR, IL3R, fibroblast activating protein (FAP), Carboanhydrase IX (MN/CA IX), Carcinoembryonic antigen (CEA), EpCAM, CDCP1, Derlin1, Tenascin, frizzled 1-10, VEGFR2 (KDR/FLK1), VEGFR3 (FLT4, CD309), Endoglin, CLEC14, Tem1-8, or Tie2.

56. The isolated EV of claim 50, wherein the immune component comprises an immune cell specifically recognizing and binding to the neoantigen, an antibody specifically recognizing and binding to the neoantigen, or both.

57. The isolated EV of claim 56, wherein the immune cell comprises one or more of: an NK cell, an NKT cell, a T cell, a B cell, a dendritic cell, a splenocyte or a macrophage.

58. The isolated EV of claim 56 or 57, wherein the immune cell expressing a chimeric antigen receptor (CAR) specifically recognizing and binding to the neoantigen.

59. The isolated EV of claim 56, wherein the antibody is an immune cell engager specifically recognizing and binding to the neoantigen and a cell surface marker of the immune cell.

60. The isolated EV of claim 59, wherein the cell surface marker of the immune cell comprises one or more of CD3, CD28, 4-1BB, CD16, NKG2D or CD64.

61. The isolated EV of any of claims 37 to 60, wherein the cancer cells are isolated from a tumor biopsy from the subject.

62. The isolated EV of any of claims 37 to 61, wherein the cancer cell is a mammalian cell.

63. The isolated EV of claim 62, wherein the mammal is selected from a canine, a feline, an equine, or a human patient.

64. The isolated EV of any of claims 37 to 63, wherein the cancer cell is a carcinoma cell or a sarcoma cell.

65. The isolated EV of any of claims 37 to 63, wherein the cancer cell is a cell of a solid tumor, optionally a brain tumor, and further optionally a glioma.

66. A population of isolated EVs of any of claims 37 to 65.

67. A composition comprising the isolated EV of any of claims 37 to 65 or the population of claim 66, and a carrier.

68. The composition of claim 67, wherein the carrier is a pharmaceutically acceptable carrier.

69. The composition of claim 67 or 68, wherein the EVs are the same or different from each other.

70. The composition of any one of claims 67 to 69, further comprising an anti-cancer agent.

71. A kit comprising one or more of: the isolated EV of any one of claims 37 to 65, the population to claim 66, or the composition of any one of claims 67 to 70, and instructions for use.
